(19) United States
(12) Patent Application Publication
(10) Pub. No.: US 2025/0325587 A1
(43) Pub. Date: Oct. 23, 2025

(54) BLOCKADE OF CD3 EXPRESSION AND CHIMERIC ANTIGEN RECEPTORS FOR IMMUNOTHERAPY

(71) Applicant: MediSix Therapeutics, Inc., Helios (SG)

(72) Inventors: Hongliang QIAN, Singapore (SG); Dario CAMPANA, Singapore (SG); Ying Xim TAN, Singapore (SG)

(21) Appl. No.: 19/122,674

(22) PCT Filed: Nov. 1, 2023
(86) PCT No.: PCT/US2023/078411

§ 371 Date: Apr. 18, 2025

Related U.S. Application Data
(60) Provisional application No. 63/421,913, filed on Nov. 2, 2022.
(60) Provisional application No. 63/529,047, filed on Jul. 26, 2023.

(51) Int. Cl. A61K 35/17; A61K 40/11; A61K 40/31; A61K 40/42; A61P 35/02; C07K 14/525; C07K 14/55; C07K 14/57; C07K 14/705; C07K 16/28; C12N 5/00; C12N 5/0783

(52) U.S. Cl. CPC A61K 35/17; A61K 40/11; A61K 40/31; A61K 40/421; A61P 35/02; C07K 14/525; C07K 14/55; C07K 14/57; C07K 14/705; C07K 16/2809; C07K 16/2887; C12N 5/0018; C12N 5/0636; A61K 2239/13; A61K 2239/21; A61K 2239/22; A61K 2239/25; A61K 2239/48; C07K 2317/31; C07K 2317/565; C07K 2319/03; C07K 2319/05; C12N 2501/51; C12N 2501/515; C12N 2539/00

(57) ABSTRACT

T cells expressing a chimeric antigen receptor (CAR) targeting CD3 can be susceptible to fratricide because T cells express CD3 on their surface as part of the T cell receptor (TCR)/CD3 complex. To reduce fratricide, CD3 surface expression can be downregulated using an anti-CD3 antibody (e.g., an anti-CD3 single-chain antibody) coupled to an intracellular targeting signal such as an endoplasmic reticulum (ER) retention signal. Retention of CD3 in the ER can allow T cells expressing a CD3 CAR to grow in culture without compromising their cytotoxic activity against CD3 positive T cells. The T cells described herein can be particularly useful for treating T cell diseases (e.g., a disease caused by T cell defects or disorders). In addition, downregulating CD3 surface expression can reduce graft versus host disease when allogeneic T cells are introduced into a mammalian host.

### CROSS-REFERENCE TO RELATED APPLICATIONS

This application claims the benefit of U.S. Provisional Application Ser. No. 63/529,047, filed on Jul. 26, 2023, and U.S. Provisional Application Ser. No. 63/421,913, filed on Nov. 2, 2022, the entire content of each of which is incorporated herein by reference in their entirety.

### REFERENCE TO A SEQUENCE LISTING

The instant application contains a Sequence Listing which has been submitted electronically in XML file format and is hereby incorporated by reference in its entirety. Said XML copy, created on Oct. 30, 2023, is named 62190-726_601_SL.xml and is 163,074 bytes in size.

### BACKGROUND OF THE DISCLOSURE

Chimeric antigen receptors (CARs) are artificial hybrid proteins that can redirect immune cells and activate them upon engagement of a specifically recognized target molecule. CARs are now widely used to endow immune cells, such as T lymphocytes or natural killer cells, with the capacity to kill cancer cells. CARs are typically constituted by a single-chain variable region (scFv) of an antibody linked to a signaling region via a transmembrane domain. When the scFv binds to the corresponding antigen expressed on the surface of target cells, signal transduction is triggered and the process of killing the target cell initiates. Clinical trials with CAR-expressing T lymphocytes targeting CD19 and other B-cell associated antigens have shown remarkable responses in patients with B-cell or plasma cell malignancies, such as acute lymphoblastic leukemia (ALL), non-Hodgkin lymphoma (NHL) and multiple myeloma.

In comparison with the progress made with CAR-T cell therapies in B-cell malignancies, the development of similar technologies to target T-cell and NK-cell malignancies, e.g., Peripheral T-cell lymphoma (PTCL) has lagged behind. Cluster of Differentiation 3 (CD3) is an attractive target because it remains highly expressed in many T-cell malignancies and its expression is specific to the hematological compartment. However, expression of an anti-CD3 CAR (“CD3 CAR”) in T cells results in self-killing, precluding manufacturing of sufficient CAR-T cell numbers. Novel therapies for T-cell malignancies are needed but progress to date has been slow.

In sum, there is a significant unmet need for new therapeutic options for patients with T-cell malignancies.

### SUMMARY OF THE DISCLOSURE

Recognized herein is a need for improved CAR-T cell therapies and methods of producing the engineered CAR-T cells. The compositions and methods provided herein can produce engineered CAR-T cells and eliminate CAR-mediated self-killing or fratricide of the T cells. The compositions and methods provided herein can also reduce the risk of developing graft-versus-host-disease (GvHD).

In one aspect, the present disclosure provides an immune cell comprising: a first polynucleotide comprising a nucleotide sequence encoding a protein expression blocker (PEBL) and a second polynucleotide comprising a nucleotide sequence encoding a chimeric antigen receptor (CAR), wherein the PEBL comprises a first CD3-binding domain coupled to an intracellular targeting signal, wherein the intracellular targeting signal comprises an endoplasmic reticulum (ER) retention sequence, a Golgi retention sequence, or a proteosome localizing sequence, wherein the PEBL downregulates cell surface expression of endogenous CD3 in the immune cell, wherein the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif, wherein the first CD3-binding domain and the second CD3-binding domain each comprises six complementarity determining regions (CDRs) from heavy chain (HC) and light chain (LC) variable domains of a UCHT1 antibody, and wherein: HC CDR1 comprises SEQ ID NO: 13 or SEQ ID NO: 31, HC CDR2 comprises SEQ ID NO: 14 or SEQ ID NO: 32, HC CDR3 comprises SEQ ID NO: 15 or SEQ ID NO: 33, LC CDR1 comprises SEQ ID NO: 22 or SEQ ID NO: 40, LC CDR2 comprises SEQ ID NO: 23 or SEQ ID NO: 41, and LC CDR3 comprises SEQ ID NO: 24 or SEQ ID NO: 42.

In some embodiments, the first CD3-binding domain and the second CD3-binding domain each comprises a heavy chain variable (VH) domain comprising a sequence having at least 90% identity to SEQ ID NO: 1 and a light chain variable (VL) domain comprising a sequence having at least 90% identity to SEQ ID NO: 2. In some embodiments, the first CD3-binding domain and the second CD3-binding domain each comprises a VH domain comprising a sequence of SEQ ID NO: 1 and a VL domain comprising a sequence of SEQ ID NO: 2.

In another aspect, the present disclosure provides an immune cell comprising: a first polynucleotide comprising a nucleotide sequence encoding a protein expression blocker (PEBL) and a second polynucleotide comprising a nucleotide sequence encoding a chimeric antigen receptor (CAR), wherein the PEBL comprises a first CD3-binding domain coupled to an intracellular targeting signal, wherein the intracellular targeting signal comprises an endoplasmic reticulum (ER) retention sequence, a Golgi retention sequence, or a proteosome localizing sequence, wherein the PEBL downregulates cell surface expression of endogenous CD3 in the immune cell, wherein the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif, wherein the first CD3-binding domain comprises six complementarity determining regions (CDRs) from heavy chain (HC) and light chain (LC) variable domains of a UCHT1 antibody, wherein: HC CDR1 comprises SEQ ID NO: 13 or SEQ ID NO: 31, HC CDR2 comprises SEQ ID NO: 14 or SEQ ID NO: 32, HC CDR3 comprises SEQ ID NO: 15 or SEQ ID NO: 33, LC CDR1 comprises SEQ ID NO: 22 or SEQ ID NO: 40, LC CDR2 comprises SEQ ID NO: 23 or SEQ ID NO: 41, and LC CDR3 comprises SEQ ID NO: 24 or SEQ ID NO: 42; and wherein the second CD3-binding domain comprises six complementarity determining regions (CDRs) from heavy chain (HC) and light chain (LC) variable domains of a 28F11 antibody, wherein: HC CDR1 comprises SEQ ID NO: 19 or SEQ ID NO: 37, HC CDR2 comprises SEQ ID NO: 20 or SEQ ID NO: 38, HC CDR3 comprises SEQ ID NO: 21 or SEQ ID NO: 39, LC CDR1 comprises SEQ ID NO: 28 or SEQ ID NO: 46, LC CDR2 comprises SEQ ID NO: 29 or SEQ ID NO: 47, and LC CDR3 comprises SEQ ID NO: 30 or SEQ ID NO: 48.

In some embodiments, the first CD3-binding domain and the second CD3-binding domain each comprises a heavy chain variable (VH) domain comprising a sequence having at least 90% identity to SEQ ID NO: 5 and a light chain variable (VL) domain comprising a sequence having at least 90% identity to SEQ ID NO: 6.

In some embodiments, the first CD3-binding domain and the second CD3-binding domain each comprises a VH domain comprising a sequence of SEQ ID NO: 5 and a VL domain comprising a sequence of SEQ ID NO: 6.

In some embodiments, the PEBL further comprises a linker sequence between the first CD3-binding domain and the intracellular targeting signal.

In some embodiments, the linker sequence comprises a sequence of SEQ ID NO: 135 or SEQ ID NO: 139.

In some embodiments, the ER retention sequence comprises KDEL (SEQ ID NO: 137), KKXX or KXD/E, wherein X is any amino acid.

In some embodiments, the ER retention sequence comprises SEQ ID NO: 142. In some embodiments, the ER retention sequence comprises SEQ ID NO: 143. In some embodiments, the Golgi retention sequence comprises YQRL (SEQ ID NO: 147).

In some embodiments, the transmembrane domain comprises SEQ ID NO: 100. In some embodiments, the costimulatory domain comprises SEQ ID NO: 102. In some embodiments, the cytoplasmic signaling domain comprises SEQ ID NO: 103.

In some embodiments, the second polynucleotide further comprises a kill gene. In some embodiments, the kill gene comprises a sequence encoding CD20 or a truncated fragment thereof. In some embodiments, the CD20 fragment comprises SEQ ID NO: 124.

In some embodiments, the second polynucleotide comprises an internal ribosome entry site between the nucleotide sequence encoding the CAR and the sequence encoding the kill gene. In some embodiments, the second polynucleotide comprises a nucleotide sequence encoding a self-cleaving peptide between the nucleotide sequence encoding the CAR and the sequence encoding the kill gene. In some embodiments, the second polynucleotide comprises, in the 5′ to 3′ direction, the sequence encoding the CAR, the sequence encoding a self-cleaving peptide, and the sequence encoding a truncated CD20 fragment.

In some embodiments, the self-cleaving peptide comprises a P2A sequence. In some embodiments, the self-cleaving peptide further comprises a linker sequence. In some embodiments, the linker sequence comprises GSG. In some embodiments, the self-cleaving peptide comprises SEQ ID NO: 122.

In some embodiments, the immune cell further comprises a third polynucleotide further comprises a kill gene. In some embodiments, the kill gene comprises a sequence encoding CD20 or a truncated fragment thereof. In some embodiments, the CD20 fragment comprises SEQ ID NO: 124.

In some embodiments, the first polynucleotide further comprises a nucleotide sequence encoding a CD8 signal peptide operably linked to the nucleotide sequence encoding the PEBL.

In some embodiments, the first polynucleotide further comprises an MSCV promoter operably linked to the nucleotide sequence encoding the PEBL. In some embodiments, the second polynucleotide further comprises an MSCV promoter operably linked to the nucleotide sequence encoding the CAR. In some embodiments, the nucleotide sequence encoding a PEBL comprises a codon optimized sequence.

In another aspect, the present disclosure provides an immune cell comprising: a first polynucleotide comprising a nucleotide sequence encoding a protein expression blocker (PEBL) and a second polynucleotide comprising a nucleotide sequence encoding a chimeric antigen receptor (CAR) and a nucleotide sequence encoding a kill gene, wherein the PEBL comprises a first CD3-binding domain coupled to an intracellular targeting signal, wherein the intracellular targeting signal comprises an endoplasmic reticulum (ER) retention sequence, a Golgi retention sequence, or a proteosome localizing sequence, and wherein the PEBL downregulates cell surface expression of endogenous CD3 in the immune cell, and wherein the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif.

In some embodiments, the kill gene comprises a sequence encoding CD20 or a truncated fragment thereof. In some embodiments, the CD20 fragment comprises SEQ ID NO: 124.

In some embodiments, the second polynucleotide comprises an internal ribosome entry site between the nucleotide sequence encoding the CAR and the kill gene. In some embodiments, the second polynucleotide comprises a nucleotide sequence encoding a self-cleaving peptide between the nucleotide sequence encoding the CAR and the nucleotide sequence encoding the kill gene. In some embodiments, the second polynucleotide comprises, in the 5′ to 3′ direction, the sequence encoding the CAR, the sequence encoding a self-cleaving peptide, and the sequence encoding a truncated CD20 fragment.

In some embodiments, the self-cleaving peptide comprises GSG-P2A. In some embodiments, the self-cleaving peptide comprises SEQ ID NO: 122.

In some embodiments, the first CD3-binding domain and the second CD3-binding domain are the same CD3-binding domain.

In some embodiments, the immune cell secretes one or more cytokines in the presence of CD3+ cells. In some embodiments, the one or more cytokines comprises IFNγ, TNFα, and/or IL-2. In some embodiments, the immune cell does not mediate xenoreactivity when administered into a subject in need thereof.

In some embodiments, the immune cell does not mediate alloreactivity when administered into a subject in need thereof.

In another aspect, the present disclosure provides a cell population comprising the immune cell described herein.

In some embodiments, the cell population comprises peripheral blood mononuclear cells. In some embodiments, at least 80% of the cells of the cell population are T cells. In some embodiments, at least 40% of the cells of the cell population are CD4-positive T cells. In some embodiments, at least 40% of the cells of the cell population are CD8-positive T cells.

In some embodiments, at least 80% of cells of the cell population have at least 10-fold reduced cell surface expression of CD3 compared to an otherwise identical cell population that does not comprise an immune cell comprising the first polynucleotide encoding the PEBL.

In some embodiments, at least 30% of cells of the cell population express the CAR.

In some embodiments, at least 50% of cells of the cell population have at least 10-fold reduced cell surface expression of CD3 compared to an otherwise identical cell population that does not comprise an immune cell comprising the first polynucleotide encoding the PEBL and express the CAR.

In some embodiments, the cell population is capable of at least 10-fold expansion in 10 days. In some embodiments, the cells of the cell population having at least 10-fold reduced cell surface expression of CD3 compared to an otherwise identical cell population that does not comprise an immune cell comprising the first polynucleotide encoding the PEBL and expressing the CAR are capable of at least 20-fold expansion in 10 days. In some embodiments, at least 20% of the cells of the cell population are CAR+CD20+ cells, and wherein the CAR+CD20+ cells are susceptible to antibody-dependent cellular cytotoxicity mediated by NK effector cells expressing a chimeric receptor with an extracellular CD16 Fc binding domain, a transmembrane domain, and a cytoplasmic domain with a 4-1BB costimulatory domain and a CD3ζ primary signaling domain at an effector to target ratio of at least 1:1 during a 48 hour incubation in the presence of 1 μg/ml rituximab. In some embodiments, at least 50% of the cells of the cell population are CAR+CD20+ cells, and wherein the CAR+CD20+ cells are susceptible to antibody-dependent cellular cytotoxicity mediated by NK effector cells expressing a chimeric receptor with an extracellular CD16 Fc binding domain, a transmembrane domain, and a cytoplasmic domain with a 4-1BB costimulatory domain and a CD3ζ primary signaling domain at an effector to target ratio of at least 1:1 during a 48 hour incubation in the presence of 1 μg/ml rituximab. In some embodiments, at least 20% of the cells of the cell population are CAR+CD20+ cells, and wherein the CAR+CD20+ cells are susceptible to complement-dependent cytotoxicity mediated by 25% (v/v) baby rabbit complement in the presence of at least 1 μg/ml rituximab. In some embodiments, at least 80% of the cells of the cell population are CAR+CD20+ cells, and wherein the CAR+CD20+ cells are susceptible to complement-dependent cytotoxicity mediated by 25% (v/v) baby rabbit complement in the presence of at least 1 μg/ml rituximab.

In another aspect, the present disclosure provides a method of manufacturing a cellular composition comprising: obtaining a cell population comprising immune cells, introducing a first polynucleotide into the immune cells, and introducing a second polynucleotide into the immune cells at least about two days after introducing the first polynucleotide into the immune cells, wherein the first polynucleotide encodes a protein expression blocker (PEBL), and the second polynucleotide encodes a chimeric antigen receptor (CAR).

In some embodiments, the PEBL comprises a first CD3-binding domain coupled to an intracellular targeting signal. In some embodiments, the intracellular targeting signal comprises an ER retention sequence, a Golgi retention sequence, or a proteosome localizing sequence. In some embodiments, the PEBL further comprises a linker sequence between the first CD3-binding domain and the intracellular targeting signal.

In some embodiments, the linker sequence comprises a sequence of SEQ ID NO: 135. In some embodiments, the ER retention sequence comprises KDEL (SEQ ID NO: 137), KKXX or KXD/E, wherein X is any amino acid. In some embodiments, the ER retention sequence comprises SEQ ID NO: 142. In some embodiments, the ER retention sequence comprises SEQ ID NO: 143. In some embodiments, the Golgi retention sequence comprises YQRL (SEQ ID NO: 147).

In some embodiments, the PEBL downregulates cell surface expression of endogenous CD3 in the immune cells.

In some embodiments, the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif.

In some embodiments, the transmembrane domain comprises SEQ ID NO: 100. In some embodiments, the costimulatory domain comprises SEQ ID NO: 102. In some embodiments, the cytoplasmic signaling domain comprises SEQ ID NO: 103.

In some embodiments, the first CD3-binding domain or the second CD3-binding domain binds to CD3γ or CD3δ. In some embodiments, the first CD3-binding domain or the second CD3-binding domain binds to CD3ε.

In some embodiments, the first CD3-binding domain and the second CD3-binding domain each comprise six complementarity determining regions (CDRs) from heavy chain (HC) and light chain (LC) variable domains of a UCHT1 antibody, wherein: HC CDR1 comprises SEQ ID NO: 13 or SEQ ID NO: 31, HC CDR2 comprises SEQ ID NO: 14 or SEQ ID NO: 32, HC CDR3 comprises SEQ ID NO: 15 or SEQ ID NO: 33, LC CDR1 comprises SEQ ID NO: 22 or SEQ ID NO: 40, LC CDR2 comprises SEQ ID NO: 23 or SEQ ID NO: 41, and LC CDR3 comprises SEQ ID NO: 24 or SEQ ID NO: 42.

In some embodiments, the first CD3-binding domain and the second CD3-binding domain each comprise sequences having at least 90% identity to SEQ ID NO: 1 and SEQ ID NO: 2. In some embodiments, the first CD3-binding domain and the second CD3-binding domain each comprise SEQ ID NO: 1 and SEQ ID NO: 2.

In some embodiments, the first CD3-binding domain comprises six complementarity determining regions (CDRs) from heavy chain (HC) and light chain (LC) variable domains of a UCHT1 antibody, wherein: the first CD3-binding domain comprises six complementarity determining regions (CDRs) from heavy chain (HC) and light chain (LC) variable domains of a UCHT1 antibody, wherein: HC CDR1 comprises SEQ ID NO: 13 or SEQ ID NO: 31, HC CDR2 comprises SEQ ID NO: 14 or SEQ ID NO: 32, HC CDR3 comprises SEQ ID NO: 15 or SEQ ID NO: 33, LC CDR1 comprises SEQ ID NO: 22 or SEQ ID NO: 40, LC CDR2 comprises SEQ ID NO: 23 or SEQ ID NO: 41, and LC CDR3 comprises SEQ ID NO: 24 or SEQ ID NO: 42; and the second CD3-binding domain comprises six complementarity determining regions (CDRs) from heavy chain (HC) and light chain (LC) variable domains of a 28F11 antibody, wherein: HC CDR1 comprises SEQ ID NO: 19 or SEQ ID NO: 37, HC CDR2 comprises SEQ ID NO: 20 or SEQ ID NO: 38, HC CDR3 comprises SEQ ID NO: 21 or SEQ ID NO: 39, LC CDR1 comprises SEQ ID NO: 28 or SEQ ID NO:46, LC CDR2 comprises SEQ ID NO: 29 or SEQ ID NO: 47, and LC CDR3 comprises SEQ ID NO: 30 or SEQ ID NO: 48.

In some embodiments, the first CD3-binding domain and the second CD3-binding domain each comprise sequences having at least 90% identity to SEQ ID NO: 5 and SEQ ID NO: 6. In some embodiments, the first CD3-binding domain and the second CD3-binding domain each comprise SEQ ID NO: 5 and SEQ ID NO: 6.

In some embodiments, the second polynucleotide further comprises a kill gene. In some embodiments, the kill gene encodes CD20 or a truncated fragment thereof. In some embodiments, the CD20 fragment comprises SEQ ID NO: 124.

In some embodiments, a retroviral vector comprises the first polynucleotide. In some embodiments, a retroviral vector comprises the second polynucleotide. In some embodiments, a lentiviral vector comprises the first polynucleotide. In some embodiments, a lentiviral vector comprises the second polynucleotide. In some embodiments, the cell population comprises peripheral blood mononuclear cells.

In some embodiments, at least 80% of the cells of the immune cell population are T cells. In some embodiments, at least 40% of the cells of the immune cell population are CD4-positive T cells. In some embodiments, at least 40% of the cells of the immune cell population are CD8-positive T cells.

In some embodiments, the second polynucleotide is introduced about two days after introducing the first polypeptide into the immune cells. In some embodiments, the immune cells are activated before the introducing a first polynucleotide into the immune cells. In some embodiments, the activating comprises contacting the immune cells with an anti-CD3 antibody and an anti-CD28 antibody. In some embodiments, the activating comprises contacting the immune cells with a polymeric nanomatrix conjugated to anti-CD3 antibodies and anti-CD28 antibodies.

In some embodiments, the method further comprises culturing the cells in a growth media, wherein the cell population expands at least 10-fold after 10 or less days of growth. In some embodiments, the culturing comprising contacting the cells to a gas permeable membrane.

In some embodiments, the method further comprises harvesting the cell population and cryopreserving the cell population.

In another aspect, the present disclosure provides a cell population manufactured by the method described herein.

In another aspect, the present disclosure provides a therapeutic composition comprising the cell population described herein and a pharmaceutically acceptable excipient.

In another aspect, the present disclosure provides a method of treating a T cell disease in a subject comprising administering the therapeutic composition described herein to the subject.

In some embodiments, the T cell disease comprises a T cell lymphoma. In some embodiments, the T cell lymphoma comprises peripheral T cell lymphoma. In some embodiments, the T cell disease comprises a T cell leukemia. In some embodiments, the T cell disease comprises an autoimmune disease. In some embodiments, graft versus host disease in the subject is reduced compared to an identical subject treated with a therapeutic composition comprising a cell population comprising immune cells expressing the CAR and not expressing the PEBL.

In some embodiments, the method further comprises administering a trigger that activates the kill gene. In some embodiments, the trigger that activates the kill gene comprises an anti-CD20 antibody. In some embodiments, the anti-CD20 antibody comprises rituximab.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”, “Fig.”, and “FIGURE” herein) of which:

FIG. 1 illustrates a CD3 PEBL CD3 CAR T cells manufacturing process with sequential transduction.

FIG. 2 shows that CD3 PEBL CD3 CAR T cells generated via sequential transduction have high CAR expression and low CD3 expression as determined by flow cytometry. T cells were co-transduced with lentiviral vectors delivering an anti-CD3 CAR and an anti-CD3 PEBL construct (PEBL-CAR) or transduced with only anti-CD3 PEBL lentiviral vector. Non-transduced T cells (Ntx) are CD3+ and lack CAR expression.

FIG. 3A illustrates a bicistronic lentiviral vector for simultaneous expression of CD3 PEBL and CD3 CAR with an internal ribosome entry site (IRES) between sequences encoding the PEBL and CAR.

FIG. 3B shows that T cells transduced with the bicistronic construct have low CAR expression and high CD3 expression.

FIG. 4A compares expression of the indicated CARs and function of the indicated PEBLs after Day1, Day3 sequential transduction. Flow cytometry was performed on Day 6.

FIG. 4B compares CAR and GFP expression in the same cells depicted in FIG. 4A. Each CAR was expressed from a bicistronic vector comprising a P2A self-cleaving linker separating DNA encoding the CAR from DNA encoding GFP.

FIG. 5 compares cytotoxic activity of the indicated CARs in CD3 (UCHT1) PEBL CD3 (OKT3, UCHT1 or 28F11)/TCR (CIV3) CAR T cells generated by sequential transduction of PBMCs against Jurkat (CD3+) or Nalm6 (CD3−) target cells at an effector:target ratio of 1:10. Cytotoxicity was quantified 24 hours after co-culture of effector and target cells.

FIG. 6 shows that sequential transduction of the CD3 PEBL and CD3 CAR vectors on Day 1 and Day 3 (D1D3) yields higher CAR expression than sequential transduction on Day 1 and Day 2 (D1D2). Flow cytometry was performed on Day 6.

FIG. 7A shows in vitro cytotoxicity of CD3 (UCHT1) PEBL CD3 (UCHT1) CAR T cells against Jurkat-GFP (CD3+) or Nalm6-GFP (CD3−) target cells. GFP intensity was assessed as a proxy for viable target cell number. Non-transduced T cells (Ntx) were tested as a control.

FIG. 7B shows proliferation of the CD3 (UCHT1) PEBL CD3 (UCHT1) CAR T cells induced by co-culture with irradiated Jurkat (CD3+) or Nalm6 (CD3−) cells.

FIG. 8 shows treatment of Jurkat (CD3+) xenograft leukemia by CD3 (UCHT1) PEBL CD3 (UCHT1) CAR T cells.

FIG. 9 shows percentage change in body weight of immunodeficient mice infused with CD3 (UCHT1) PEBL CD3 (UCHT1) CAR T cells, CD3 (UCHT1) PEBL only T cells or non-transduced (Ntx) T cells over time.

FIG. 10 illustrates various constructs for expressing the CD20 epitope of rituximab in combination with a CD3 CAR. Constructs 1-3 incorporated a truncated CD20. Construct 4 incorporated a series of three CD20 mimotopes.

FIG. 11 shows the expression of CD3 CAR and CD20 epitopes in CD3 knockout Jurkat cells transduced with the constructs depicted in FIG. 10.

FIG. 12 shows expression of CAR, CD20 and CD3 in T cells transduced with CD3 PEBL and the CD3 CAR/CD20t constructs of FIG. 10.

FIG. 13A and FIG. 13B show in vitro cytotoxicity mediated by CD3 PEBL T cells transduced with the CD3 CAR/CD20t constructs of FIG. 10 on Jurkat-GFP (CD3+) target cells (FIG. 13A) and Nalm6-GFP (CD3−) target cells (FIG. 13B) at an effector to target (E:T) ratio of 1:10.

FIG. 14 shows secretion of cytokines IFNγ, TNFα, and TL-2 by CD3 PEBL CD3 CAR-CD20t T cells in the presence of CD3+ target cells.

FIG. 15 shows that CD3 PEBL CD3 CAR-CD20t T cells are susceptible to killing in the presence of rituximab but not trastuzumab in an antibody dependent cellular cytotoxicity (ADCC) assay.

FIG. 16 shows that CD3 PEBL CD3 CAR-CD20t T cells are susceptible to killing in the presence of rituximab but not trastuzumab in a complement-dependent cytotoxicity (CDC) assay.

FIG. 17A and FIG. 17B show depletion of CD3 PEBL CD3 CAR-CD20t T cells by rituximab in mice. FIG. 17A shows the experimental protocol and timeline for rituximab administration (with human IgG as control). FIG. 17B shows quantification of CD3 PEBL CD3 CAR-CD20t T cell number in mouse peripheral blood by flow cytometry.

FIG. 18A and FIG. 18B show expression of anti-CD3 CAR in T cells leads to fratricide in the absence of anti-CD3 PEBL. FIG. 18A shows flow cytometric analysis of CAR and CD20t after transduction with anti-CD3 CAR-CD20t lentiviral vector. FIG. 18B shows cell counts of nontransduced cells and cells transduced with anti-CD3 CAR-CD20t during manufacturing.

FIG. 19A, FIG. 19B, and FIG. 19C show CD3 PEBL CD3 CAR-CD20t T cells do not mediate xenoreactivity in mice. Immunodeficient mice were irradiated and infused with non-transtransduced or transduced T cells or left untreated. Each line on the graphs represents the data from one mouse. FIG. 19A shows percentage change in weight over time. FIG. 19B shows quantified platelet counts over time. FIG. 19C shows quantified hemoglobin levels over time. An X denotes a mouse euthanized due to greater than 20% weight loss. Mice were grouped in five conditions: irradiated control (untreated), non-transduced (NTX) T cells, T cells transduced with eGFP, T cells transduced with anti-CD3 PEBL only, or T cells transduced with CD3 PEBL CD3 CAR-CD20t.

FIG. 20A and FIG. 20B show tumor killing activity of CD3 PEBL CD3 CAR-CD20t T cells in a tumor xenograft model. Immunodeficient mice were infused with CD3+tumor cells expressing firefly luciferase and subsequently treated with non-transduced or transduced T cells. FIG. 20A shows bioluminescence images of mice over time in four conditions: irradiated control (untreated), non-transduced (NTX) T cells, treatment with CD20t transduced T cells, or treatment with CD3 PEBL CD3 CAR-CD20t T cells. FIG. 20B shows the change in total flux (photons per second) for each mouse over time.

### DETAILED DESCRIPTION OF THE DISCLOSURE

### Definitions

Unless defined otherwise, all technical and scientific terms used herein have the same meaning as commonly understood by one of ordinary skill in the art to which the disclosure pertains.

The term “a” and “an” as used herein refer to one or to more than one (i.e., to at least one) of the grammatical object of the article, e.g., “an element” means one element or more than one element.

The term “about” or “approximately” as used herein refers to a measurable value such as an amount, a temporal duration, and the like, refers to being within a statistically meaningful range of variations of 20% or in some instances ±10%, or in some instances ±5%, or in some instances ±1%, or in some instances ±0.1% from the specified value.

The term “alleviate” as used herein, in context to a disease refers to reducing the severity of one or more symptoms of the disease.

The term “allogeneic”, as used herein, refers to any material derived from an individual that is transplanted into a genetically different recipient of the same species. Two or more individuals are said to be allogeneic to one another when the genes at one or more loci are not identical. In some aspects, allogeneic material from individuals of the same species may be sufficiently unlike genetically to interact antigenically.

The term “autologous” as used herein refers to any material derived from the same individual to which it is later to be re-introduced into the individual.

The term “binding domain” as used herein (e.g., “CD3 binding domain”) refers to a protein having affinity to a target. For example, the binding domain can be an immunoglobulin chain or fragment thereof, comprising at least one immunoglobulin variable domain sequence. The binding domain can be antibodies or antibody fragments. In some embodiments, the binding domain can be a portion of a receptor that can bind to a ligand (e.g., a ligand-binding domain of a receptor) or a ligand of a receptor.

The terms “binds” or “specifically binds,” as used herein with respect to an antibody, refers to an antibody which recognizes a specific antigen, but does not substantially recognize or bind other molecules in a sample. For example, an antibody that specifically binds to an antigen from one species may also bind to that antigen from one or more species. However, such cross-species reactivity does not itself alter the classification of an antibody as specific. In another example, an antibody that specifically binds to an antigen may also bind to different allelic forms of the antigen. Thus, under designated immunoassay conditions, the specified antibodies bind to a particular protein at least two times the background and typically more than 10 to 100 times the background. Specific binding to an antigen under such conditions requires an antibody that is selected for its specificity for a particular protein.

The term “antibody” as used herein refers to a protein, or polypeptide sequence derived from an immunoglobulin molecule which specifically binds to an antigen. Antibodies can be polyclonal or monoclonal, multiple or single chain, or intact immunoglobulins, and may be derived from natural sources or from recombinant sources. In some embodiments, an antibody comprises at least one heavy chain and one light chain. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). An “antibody” includes, but are not limited to, monoclonal, polyclonal, bispecific, multispecific, murine, chimeric, camelid VHH, humanized and human antibodies. In some embodiments, the antibody disclosed herein is a CD3 antibody, e.g., a CD3P antibody, e.g., an UCHT 1 antibody.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. In some embodiments, the antibody disclosed herein is a CD3 scFv, e.g., a CD3ε scFv, e.g., an UCHT1 scFv.

In some embodiments, an antibody can be modified or engineered, e.g., chimeric antibodies, humanized antibodies, multiparatopic antibodies (e.g., biparatopic antibodies), and multispecific antibodies (e.g., bispecific antibodies). In some embodiments, an antibody can be a nanobody-based heavy chain antibody engineered and/or modified from an immunoglobulin.

The term “variable region” or “variable domain” refers to the domain of an antibody heavy or light chain that is involved in binding the antibody to antigen. The term “antibody heavy chain,” (VH) refers to the larger of the two types of polypeptide chains present in antibody molecules in their naturally occurring conformations, and which normally determines the class to which the antibody belongs. The term “antibody light chain,” (VL) refers to the smaller of the two types of polypeptide chains present in antibody molecules in their naturally occurring conformations. Kappa (K) and lambda (k) light chains refer to the two major antibody light chain isotypes. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. The VH and VL regions can be further subdivided into regions of hypervariability, termed hypervariable region (HVR) or complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, these CDRs can be distributed between their appropriate framework regions. In certain embodiments of the invention, the FRs of the antibody (or antigen binding fragment thereof) may be identical to the human germline sequences or may be naturally or artificially modified.

The term “hypervariable region,” “HVR,” “complementarity determining region” or “CDR,” as used herein, refers to the sequences of amino acids within antibody variable regions which confer antigen specificity and binding affinity. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), A1-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). Generally, antibodies comprise six HVRs: three in the VH (HCDR1, HCDR2, HCDR3), and three in the VL (LCDR1, LCDR2, LCDR3).

The positions of the CDRs and framework regions can be determined using various well-known definitions in the art, e.g., Kabat (Wu, T. T., E. A. Kabat. 1970. An analysis of the sequences of the variable regions of Bence Jones proteins and myeloma light chains and their implications for antibody complementarity. J. Exp. Med. 132; 211-250; Kabat, E. A., Wu, T. T., Perry, F L, Gottesman, K., and Foeller, C. (1991) Sequences of Proteins of Immunological Interest, 5th ed., NIH Publication No. 91-3242, Bethesda, MD), Chothia (Chothia and Lesk, J. Mol. Biol., 196:901-917 (1987); Chothia et al., Nature, 342:877-883 (1989); Chothia et al., J. Mol. Biol., 227:799-817 (1992); A1-Lazikani et al., J. Mol. Biol., 273:927-748 (1997)), ImMunoGeneTics database (IMGT) (on the worldwide web at imgt.org/; Giudicelli, V., Duroux, P., Ginestoux, C, Folch, G., Jabado-Michaloud, J., Chaume, D. and Lefranc, M.-P. IMGT/LIGM-DB, the IMGT® comprehensive database of immunoglobulin and T cell receptor nucleotide sequences Nucl. Acids Res., 34, D781-D784 (2006), PMID: 16381979; Lefranc, M.-P., Pommie, C, Ruiz, M., Giudicelli, V., Foulquier, E., Truong, L., Thouvenin-Contet, V. and Lefranc, G., IMGT unique numbering for immunoglobulin and T cell receptor variable domains and Ig superfamily V-like domains Dev. Comp. Immunol., 27, 55-77 (2003). PMID: 12477501; Brochet, X., Lefranc, M.-P. and Giudicelli, V. IMGT/V-QUEST: the highly customized and integrated system for IG and TR standardized V-J and V-D-J sequence analysis Nucl. Acids Res, 36, W503-508 (2008); AbM (Martin et al., Proc. Natl. Acad. Sci. USA, 86:9268-9272 (1989); the contact definition (MacCallum et al., J. Mol. Biol., 262:732-745 (1996)), and/or the automatic modeling and analysis tool Honegger A, Pliickthun A. (world wide web at bioc dot uzh dot ch/antibody/Numbering/index dot html).

The term “chimeric antigen receptor” or “CAR” as used herein refers to an engineered cell-surface receptor comprising, at least an extracellular antigen binding domain, a transmembrane domain and an intracellular signaling domain (maybe referred to as “cytoplasmic signaling domain”) comprising a functional signaling domain derived from a stimulatory molecule and/or a costimulatory molecule. The chimeric antigen receptors of the present invention are intended primarily for use with lymphocytes such as T cells and natural killer (NK) cells. In some embodiments, the CAR described herein is a CD3 CAR, e.g., a comprising, a binding domain that binds to an extracellular domain of CD3, a transmembrane domain and an intracellular signaling domain comprising a functional signaling domain derived from a stimulatory molecule and/or a costimulatory molecule. In some embodiments, the CD3 binding domain comprises a single-chain variable fragment antibody fragment comprising the VH and VL domains of a CD3 antibody, e.g., the VH and VL domains of a UCHT1 antibody.

The portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or a bispecific antibody.

The term “CD3” (Cluster of Differentiation 3) refers to a complex of accessory proteins (e.g., glycoproteins) associated with the T-cell receptor (TCR). CD3 proteins have an N-terminal extracellular region, a transmembrane domain and a cytoplasmic tail where the immunoreceptor tyrosine activation motifs (ITAMs) are located. CD3, e.g., mammalian CD3, e.g., human CD3 comprises a CD3γ chain, a CD3δ chain, and two CD3ε chains. The extracellular domains of CD3ε, γ and δ contain an immunoglobulin-like domain. CD3ε is a non-glycosylated polypeptide chain of 20 kDa. Both CD3γ and CD3δ are glycosylated and have a molecular weight of 25-28 kDa and 20 kDa respectively (Norman 1995). CD3ζ (also known as CD247) is a non-glycosylated polypeptide with a molecular weight of 17 kDa that shares no sequence similarity with the other CD3 polypeptide chains. CD3ζ maps to human chromosome 1 band q22-q25 (Weissman et al. 1988). CD3 eta (η) is an alternatively spliced product of the gene that encodes CD3ζ (Clayton et al. 1991) and has an apparent molecular weight of 23 kDa (Orloff et al. 1989). CD3ζ is predominantly found as a homodimer, but a fraction (approximately 10%) of CD3ζ is found complexed as a heterodimer with CD3η (Orloff et al. 1989). CD3 omega (Ω) is a non-glycosylated polypeptide chain of 28 kDa that associates with the TCR complex during TCR assembly in the ER but does not associate with the TCR complex at the cell surface (Pettey et al. 1987, Clevers et al. 1988, Neisig et al. 1993). CD3Ω might facilitate complex assembly and/or be involved in the retention of unassembled CD3 protein complexes in the endoplasmic reticulum (ER) leading to their subsequent degradation in the lysosomes.

The term “immunoreceptor tyrosine-based activation motif” or “ITAM” as used herein refers to a conserved sequence of four amino acids associated with intracellular tails of the CD3γ, CD3ε, CD3δ and CD3ζ molecules. The intracellular ITAMs are characterized by a consensus amino acid sequence of a tyrosine separated from a leucine or isoleucine by any two other amino acids (YXXL) (where X represents any amino acid residue). In some embodiments, two of the YXXL motifs are separated by between 6 and 8 amino acids in the cytoplasmic tail of the molecule (YXXL/Ix₍₆₋₈₎YxxL/I) (where X represents any amino acid residue). In some embodiments, two of the YXXL motifs are separated by one, two, three, four, five, six, seven, eight, nine, ten, eleven, or twelve amino acids in the cytoplasmic tail of the molecule. The cytoplasmic segments of CD3ε, CD3γ and CD3δ contain a single ITAM, whereas the cytoplasmic domain of the CD3ζ subunit contains three ITAMs.

The term “effective amount” as used herein refers to the minimum amount required to effect a measurable improvement of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder.

The term “express” as used herein refers to the transcription and/or translation of a particular nucleotide sequence into a protein. Proteins may be expressed and remain intracellular, become a component of the cell surface membrane, or be secreted into extracellular matrix or medium.

The term “engineered” used herein refers to any composition that has been intentionally changed by human intervention from its natural state.

An “engineered nucleic acid” as used herein refers to a nucleic acid whose sequence has been intentionally changed by human intervention to have a modification, substitution, addition, or deletion of one or more nucleotides.

The term “engineered immune cell” as used herein refers to an immune cell that has been genetically modified as compared to a naturally occurring immune cell. For example, an engineered T cell produced according to the present methods carries a nucleic acid comprising a nucleotide sequence that does not naturally occur in a T cell from which it was derived. In some embodiments, the engineered immune cell of the present invention includes a chimeric antigen receptor (CAR) and a target-binding molecule linked to a localizing domain (LD-linked target-binding molecule). In a particular embodiment, the engineered immune cell of the present invention includes a CD3 4-1BB CD3ζ CAR and a CD3 scFv linked to a localizing domain.

In certain embodiments, the engineered immune cell is an engineered T cell, an engineered natural killer (NK) cell, an engineered NK/T cell, an engineered monocyte, an engineered macrophage, or an engineered dendritic cell.

In certain embodiments, an “immune activating receptor” as used herein refers to a receptor on the surface of an immune cell that activates an immune response upon binding a cancer cell ligand. In some embodiments, the immune activating receptor comprises a molecule that, upon binding (ligation) to a ligand (e.g., peptide or antigen) expressed on a cancer cell, is capable of activating an immune response. In one embodiment, the immune activating receptor is a chimeric antigen receptor (CAR); methods for designing and manipulating a CAR are known in the art. In other embodiments, the immune activating receptor is a target-binding receptor, which is similar to a CAR, but with the scFv replaced with a target-binding molecule (e.g., a molecule that binds to CD3).

The term “fratricide” as used herein refers to when one cell in the population kills a second cell in the population wherein the first cell and the second cell are of the same type, e.g., both cells are T cells.

The term “reducing and/or preventing fratricide” as used herein relates to the decrease in the occurrence of fratricide in a population of cells as compared to a suitable control population of cells (typically, but not necessarily, a population of identical cells with normal expression of the target of a CAR).

The term “graft-versus-host disease” (GvHD) as used herein refers to the complication following an allogeneic transplant of immune cells which recognize the recipient (the host) as “foreign.” The transplanted immune cells then attack the host's body cells.

The term “identity” as used herein refers to the subunit sequence identity between two polymeric molecules particularly between two amino acid molecules, such as, between two polypeptide molecules or two polynucleotide molecules. When two amino acid sequences have the same residues at the same positions, e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions, e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. In embodiments, “percent sequence identity” means that two nucleotide sequences or two amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm can then calculate the percent sequence identity for the test sequence(s) relative to the reference sequence, based on the designated program parameters.

The term “substantially identical” means a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, at least 70%, at least 75%, at least 80%, at least 85%, at least 90%, at least 95%, at least 97%, at least 99%, at least 99.99% or 100% identical at the amino acid level or nucleic acid to the sequence used for comparison. Sequence identity is typically measured using sequence analysis software. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

The term “intracellular signaling domain,” as used herein, refers to an intracellular (e.g., cytoplasmic) portion of a molecule sufficient to transduce an effector function signal. In embodiments, the intracellular signal domain transduces the effector function signal and directs the cell to perform a specialized function. Exemplary primary intracellular signaling domains include those derived from the molecules responsible for primary stimulation, or antigen dependent simulation. In some embodiments, the intracellular signaling domain can comprise a costimulatory intracellular domain.

The term “isolated” as used herein, refers to altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment, e.g., a host cell.

The term “nucleotide sequence” in reference to a nucleic acid, refer to a contiguous series of nucleotides, e.g., a polynucleotide that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). The term “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In some embodiments, nucleic acid molecules can be modified. In the case of a double-stranded polymer, “nucleic acid” can refer to either or both strands of the molecule. In certain embodiments, the nucleotide sequence encoding, e.g., a target-binding molecule linked to a localizing domain is a heterologous sequence (e.g., a gene that is of a different species or cell type origin).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “polynucleotide” as used herein refers to a chain of nucleotides.

The term “Protein Expression Blockers” or “PEBL” as used herein refers to a polypeptide construct containing a target-binding molecule that binds a target (e.g., CD3) to be removed, neutralized or blocked linked to a domain (e.g., a localizing domain, intracellular retention domain, or a intracellular targeting signal, which terms can be used interchangeably herein) that directs the polypeptide to specific cellular compartments, such as the Golgi, endoplasmic reticulum (ER), proteasome, or cellular membrane, depending on the application. In some embodiments, a PEBL further comprises a signal peptide. In some embodiments, a PEBL further comprises a transmembrane domain. In some embodiments the transmembrane domain contributes to intracellular localization. In some embodiments, the PEBL described herein is a CD3 PEBL, e.g., a CD3-PEBL comprising a CD3-binding molecule, linked to a localizing domain or an intracellular retention domain. In some embodiments, the CD3 binding molecule comprises a single-chain variable fragment antibody fragment comprising the VH and VL domains of a CD3 antibody, e.g., the VH and VL domains of a UCHT1 antibody. “CD3 PEBL” and “anti-CD3 PEBL” can be used interchangeably in the present disclosure.

The term “host cell” as used herein refers to a cell which can support the replication or expression of the expression vector. Host cells may be prokaryotic cells such as E. coli, or eukaryotic cells, such as yeast, insect cells, amphibian cells, or mammalian cells.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. In some embodiments, the term is construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family that may be used as a gene delivery vector as described herein. Lentiviruses are unique among the retroviruses in being able to infect non-dividing cells; they can deliver a significant amount of genetic information into the DNA of the host cell. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX® vector system from Lentigen and the like. Nonclinical types of lentiviral vectors are also available and would be known to one skilled in the art.

The term “in vivo” as used herein refers to inside the body of an organism. The terms “ex vivo” or “in vitro” as used herein refer to outside the body of the organism.

The term “therapeutic protein” as used herein refers to any protein or peptide-based agent that has a therapeutic effect.

The term “subject,” as used herein, refers to any animal, e.g., a mammal or marsupial. Subjects of the present invention include but are not limited to humans, non-human primates (e.g., rhesus or other types of macaques), mice, pigs, horses, donkeys, cows, sheep, rats, and fowl of any kind.

The term “cancer” as referred herein refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma leukemia, lung cancer and the like. As used herein, the term “cancer” includes premalignant, as well as malignant cancers. In some embodiments, the cancer described herein is a stage I cancer, a stage II cancer, a stage III cancer, or a stage IV cancer.

The term “T cell disease,” as used herein, refers to a disease caused by T cell defects or disorders. A T cell disease can be a cancer caused by overgrowth of T cells. A T cell disease can be a T cell mediated disease. In some cases, the cancer is a T cell malignancy. In some cases, a T cell disease is an immune disorder.

The term “Peripheral T-Cell Lymphoma” or “PTCL” as used herein refers to a diverse group of aggressive lymphomas that develop from T-cells and/or natural killer (NK) cells. In some embodiments, the PTCL described herein is a nodal PTCL, an extranodal PTCL, or a leukemic PTCL. In some embodiments, the PTCL described herein may be aggressive (fast growing) or indolent (slow growing). In some embodiments, the “PTCL” described herein may be a PTCL-not-otherwise specified (PTCL-NOS), anaplastic large cell lymphoma (ALCL), angioimmunoblastic T-cell lymphoma (AITL), NK-/T-cell lymphoma (NKTCL) and adult T-cell leukemia/lymphoma (ATLL), enteropathy-type T-cell lymphoma, or extranodal natural killer (NK) cell/T-cell lymphoma.

The term “T cell” and its grammatical equivalents as used herein can refer to a T cell from any origin. For example, a T cell can be a primary T cell, e.g., an autologous T cell, an allogeneic T cell, a T cell line, etc. The T cell can also be human or non-human. The term “T cell activation” or “T cell triggering” and its grammatical equivalents as used herein can refer to the state of a T cell that has been sufficiently stimulated to induce detectable cellular proliferation, cytokine production and/or detectable effector function. In some cases, “full T cell activation” can be similar to triggering T cell cytotoxicity. T cell activation can be measured using various assays known in the art. Said assays can be an ELISA to measure cytokine secretion, an ELISPOT, flow cytometry assays to measure intracellular cytokine expression, flow cytometry assays to measure proliferation, and cytotoxicity assays (51Cr release assay or flow cytometry assays to enumerate live target cells) to determine target cell elimination. Said assays typically use controls (non-engineered cells) to compare to engineered cells (CAR T) to determine relative activation of an engineered cell compared to a control. Additionally, said assays can compare engineered cells incubated or put in contact with a target cell not expressing the target antigen. For example, said comparison can be a CD3 CAR T cell incubated with a target cell that does not express CD3.

As used herein, the terms “treat,” “treating,” or “treatment,” refer to counteracting a medical condition (e.g., a condition related to a T cell malignancy) to the extent that the medical condition is improved.

As used herein, “subject” refers to a mammal (e.g., human, non-human primate, cow, sheep, goat, horse, dog, cat, rabbit, guinea pig, rat, mouse). In certain embodiments, the subject is a human. A “subject in need thereof” refers to a subject (e.g., patient) who has, or is at risk for developing, a disease or condition that can be treated (e.g., improved, ameliorated, prevented) with, e.g., engineered T cells.

Provided herein are compositions of matter and methods of use for the treatment of a disease e.g., a cancer or an immune disease using cells expressing CD3 chimeric antigen receptors (CAR), e.g., CD3 CAR, optionally in combination with a protein expression blocker (PEBL) that downregulates CD3 expression on the effector T cells. Also provided herein are compositions of matter and methods of use for the treatment of a disease e.g., a cancer or an immune disease using cells expressing TCR chimeric antigen receptors (CAR), e.g., TCR CAR, optionally in combination with a protein expression blocker (PEBL) that downregulates TCR and CD3 expression on the effector T cells. A description of example embodiments of the disclosure follows.

In one aspect the disclosure provides a cell (e.g., an immune effector cell, e.g., T cell or NK cell) engineered to express a CAR, e.g., a CD3 CAR or TCR CAR, wherein the CAR-T cell (“CAR-T”) or CAR NK (“CAR-NK”) cell exhibits an antitumor property. In one aspect, a cell is transformed with the CAR and the CAR is expressed on the cell surface. In one aspect, the disclosure provides a polypeptide construct containing a target-binding molecule that binds a target (e.g., CD3, TCR) to be removed or neutralized. In one aspect, the disclosure provides a method to treat a disease, e.g., cancer in a subject in need thereof by administering a composition comprising cells expressing CD3 or TCR chimeric antigen receptors (CARs) optionally in combination with a second agent that downregulates CD3 expression on the effector T cells.

As described herein, an anti-CD3 CAR (also described herein as a CD3 CAR) induces T cells to exert specific cytotoxicity against cells expressing CD3 on their surface, e.g., T cell malignancies. Further, the health of T cells expressing a CD3 CAR was shown to be markedly improved when a CD3 CAR was used in combination with downregulation of CD3 expression on the effector T cells. Downregulation (e.g., elimination, reduction, and/or relocalization) of CD3 prevented the fratricidal effect exerted by the corresponding CD3 CAR, allowing greater T cell recovery after CAR expression as compared to cells that retained the target antigen (e.g., CD3), and a more effective cytotoxicity against T leukemia/lymphoma cells.

In one aspect, the present disclosure provides, novel nucleic acid molecules encoding chimeric antigen receptors (CARs) comprising an antibody or antibody fragment that specifically binds to CD3 (e.g., CD3 CAR), a transmembrane domain, and a signaling domain. CD3 is a type I transmembrane glycoprotein which is expressed in a proportion of T cell ALL cases as well as in mature T cell and NK cell neoplasms. In another aspect, the present disclosure provides, novel nucleic acid molecules encoding chimeric antigen receptors (CARs) comprising an antibody or antibody fragment that specifically binds to a subunit of the TCR (e.g., TCR CAR), a transmembrane domain, and a signaling domain.

The CD3 binding or TCR binding portion of the CAR and/or the CD3 binding or TCR binding portion of the PEBL (target-binding domain) may be an antibody. In some embodiments, the CD3 or TCR binding portion of the CAR or PEBL is a single-chain variable fragment (scFv) antibody fragment. In some embodiments, the antibody fragment, CD3 binding fragment, shows at least 10%, at least 20%, at least 30%, at least 40%, at least 50%, at least 60%, at least 70%, at least 80%, at least 90%, at least 95%, at least 100%, at least 120%, at least 150%, at least 200%, at least 300%, at least 400%, or at least 500% binding affinity compared to the antibody from which the antibody fragment is derived from.

In some embodiments, the antibody that binds CD3 or TCR is a single-chain variable fragment antibody (“scFv antibody”). scFv refers to antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFv, see Pluckthun (1994) The Pharmacology Of Monoclonal Antibodies, vol. 113, Rosenburg and Moore eds. Springer-Verlag, New York, pp. 269-315. See also, PCT Publication No. WO 88/01649 and U.S. Pat. Nos. 4,946,778 and 5,260,203. As would be appreciated by those of skill in the art, various suitable linkers can be designed and tested for optimal function, as provided in the art, and as disclosed herein.

In some embodiments, the CD3 antibody is a CD3ε antibody. In some embodiments, the CD3 antibody is a CD3γ antibody. In some embodiments, the CD3 antibody is a CD3δ antibody. In some embodiments, the CD3 antibody is a murine antibody. In some embodiments, the CD3 antibody is a humanized antibody. In some embodiments, the CD3 antibody is a fully human antibody.

In some embodiments, the CD3 antibody is a CD3 antibody selected from the group consisting of OKT-3, UCHT1, 28F11, HIT3a, SK7, SP34, MEM-57, 7D6, Gemtuzumab Ozogamicin, hP67.6, Lintuzumab, HuM195, AVE9633, AMG330, Muromonab, Blinatumomab, Catumaxomab, Tebentafusp, or Foralumab. In some embodiments, the CD3 antibody is UCHT1. In some embodiments, the CD3 antibody is humanized UCHT1. In some embodiments, the CD3 antibody is OKT-3. In some embodiments, the CD3 antibody is humanized OKT-3. In some embodiments, the CD3 antibody is OKT-3. In some embodiments, the CD3 antibody is 28F11. Table 1 discloses the amino acid sequences ofthe VH and VL region of exemplary anti-CD3 scFvs.

The anti-CD3 CAR comprising an anti-CD3 antibody that is a UCHT1 antibody may exhibit higher expression and higher cytotoxicity than anti-CD3 CARs comprising other antibodies when coexpressed with anti-CD3 PEBL comprising an anti-CD3 antibody that is also a UCHT1 antibody. The anti-CD3 PEBL comprising an anti-CD3 antibody that is a UCHT1 antibody may exhibit higher retention activity in reducing expression of endogenous CD3 compared with anti-CD3 PEBL comprising other antibodies.

TABLE 1
Amino acid sequences of VH regions and VL regions of exemplary anti-CD3 scFvs

| | | | SEQ |
|---|---|---|---|
| Name | Chain | Amino Acid Sequence | ID NO: |
| UCHT1 | VH | EVQLQQSGPELVKPGASMKISCKASGYSFTGYTMNWVKQSHGKN | 1 |
| | | LEWMGLINPYKGVSTYNQKFKDKATLTVDKSSSTAYMELLSLTSE | |
| | | DSAVYYCARSGYYGDSDWYFDVWGQGTTLTVFS | |
| | VL | DIQMTQTTSSLSASLGDRVTISCRASQDIRNYLNWYQQKPDGTVK | 2 |
| | | LLIYYTSRLHSGVPSKFSGSGSGTDYSLTISNLEQEDIATYFCQQGN | |
| | | TLPWTFAGGTKLEIK | |
| OKT3 | VH | EVQLQQSGAELARPGASVKMSCKASGYTFTRYTMHWVKQRPGQ | 3 |
| | | GLEWIGYINPSRGYTNYNQKFKDKATLTTDKSSSTAYMQLSSLTSE | |
| | | DSAVYYCARYYDDHYCLDYWGQGTTLTVSSA | |
| | VL | QIVLTQSPAIMSASPGEKVTMTCSASSSVSYMNWYQQKSGTSPKR | 4 |
| | | WIYDTSKLASGVPAHFRGSGSGTSYSLTISGMEAEDAATYYCQQW | |
| | | SSNPFTFGSGTKLEINR | |
| 28F11 | VH | QVQLVESGGGVVQPGRSLRLSCAASGFKFSGYGMHWVRQAPGK | 5 |
| | | GLEWVAVIWYDGSKKYYVDSVKGRFTISRDNSKNTLYLQMNSLR | |
| | | AEDTAVYYCARQMGYWHFDLWGRGTLVTVSS | |
| | VL | EIVLTQSPATLSLSPGERATLSCRASQSVSSYLAWYQQKPGQAPRL | 6 |
| | | LIYDASNRATGIPARFSGSGSGTDFTLTISSLEPEDFAVYYCQQRSN | |
| | | WPPLTFGGGTKVEIK | |

In some embodiments, the CD3 antigen binding domain is a murine scFv antibody fragment. In some embodiments, the CD3 antigen binding domain is a scFv antibody fragment that is humanized compared to the murine sequence of the scFv from which it is derived. In some embodiments, the CD3 antigen binding domain is a human scFv antibody fragment. In some embodiments, the scFv comprises an amino acid sequence as set forth in any of SEQ TD NOs: 98, 106, 109, or 112. In some embodiments, the scFv comprises an amino acid sequence having at least one, two or three modifications but not more than 30, 20 or 10 modifications of the amino acid sequence as set forth in any of SEQ ID NOs: 98, 106, 109, or 112. In some embodiments, the scFv comprises an amino acid sequence with 95-99% identity to the amino acid sequence as set forth in any of SEQ ID NOs: 98, 106, 109, or 112. In some embodiments, the scFv comprises an amino acid sequence as set forth in SEQ ID NOs:98. In some embodiments, the scFv comprises an amino acid sequence having at least one, two or three modifications but not more than 30, 20 or 10 modifications of the amino acid sequence as set forth in SEQ ID NO: 98. In some embodiments, the scFv comprises an amino acid sequence with 95-99% identity (or in some cases, at least 95%, at least 96%, at least 97%, at least 98%, or at least 99% identity) to the amino acid sequence as set forth in any of SEQ ID NOs: 98, 106, 109, or 112. In some embodiments, the scFv comprises an amino acid sequence having the heavy chain and/or light chain CDR regions of any one of SEQ ID NOs: 98, 106, 109, or 112 and with 95-99% identity to the amino acid sequence as set forth in any of SEQ ID NOs: 98, 106, 109, or 112. In some embodiments, the scFv comprises an amino acid sequence with 95-99% identity to the amino acid sequence as set forth in SEQ ID NO: 98. In some embodiments, the scFv comprises an amino acid sequence having at least one, two or three modifications but not more than 30, 20 or 10 modifications of the amino acid sequence as set forth in SEQ ID NO: 98. In some embodiments, the scFv comprises an amino acid sequence having the heavy chain and/or light chain CDR regions of SEQ ID NO: 98 and having at least one, two or three modifications but not more than 30, 20 or 10 modifications of the amino acid sequence as set forth in SEQ ID NO: 98. In some embodiments, the scFv comprises an amino acid sequence with 95-99% identity to the amino acid sequence as set forth in SEQ ID NO: 98. In some embodiments, the scFv comprises an amino acid sequence having the heavy chain and/or light chain CDR regions of SEQ ID NO: 98 and with 95-99% identity (or in some cases, at least 95%, at least 96%, at least 97%, at least 98%, or at least 99% identity) to the amino acid sequence as set forth in SEQ ID NO: 98.

In some embodiments, the CD3 binding domain comprises a heavy chain variable region (VE) comprising a heavy chain complementary determining region 1 (HC CDR1), a heavy chain complementary determining region 2 (HC CDR2), and a heavy chain complementary determining region 3 (HC CDR3) having an amino acid sequence as set forth in SEQ ID NO: 1. The CD3 binding domain can further comprise a light chain variable region (VL) comprising a light chain complementary determining region 1 (LC CDR1), a light chain complimentary determining region 2 (LC CDR2), and a light chain complementary determining region 3 (LC CDR3) of amino acid sequence as set forth in SEQ ID NO: 2 In some embodiments, the heavy chain variable region comprises an amino acid sequence with 95-99% identity to the amino acid sequence of the heavy chain variable region as set forth in SEQ ID NO. 1 and the light chain variable region comprises an amino acid sequence with 95-99%; identity to the amino acid sequence of the light chain variable region as set forth in SEQ ID NO. 2.

In certain embodiments, the anti-CD3 scFv comprises a variable heavy chain (heavy chain variable region or VH) and a variable light chain (light chain variable region or VL) having an amino acid sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 1 and 2, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO: 1. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO: 2. In some embodiments, the framework region sequence of the VH can comprise at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the framework region sequence of the VH of SEQ ID NO: 1. In some embodiments, the framework region sequence of the VL can comprise at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the framework region sequence of the VL of SEQ ID NO: 2.

In some instances, the heavy chain variable region comprises at least one (e.g., 1, 2, 3, 4, 5, 6, 7, 8, 9, 10, or more) amino acid substitution in the sequence set forth in SEQ ID NO: 1. In certain instances, the heavy chain variable region comprises 10 or fewer amino acid (e.g., 0, 1, 2, 3, 4, 5, 6, 7, 8, 9, or 10) substitutions in the sequence set forth in SEQ ID NO: 1. In some instances, the light chain variable region comprises at least one (e.g., 1, 2, 3, 4, 5, 6, 7, 8, 9, 10, or more) amino acid substitution in the sequence set forth in SEQ ID NO: 2. In certain instances, the light chain variable region comprises 10 or fewer amino acid (e.g., 0, 1, 2, 3, 4, 5, 6, 7, 8, 9, or 10) substitutions in the sequence set forth in SEQ ID NO: 2. In some instances, the heavy chain variable region comprises about one (e.g., 0, 1) amino acid substitution in one or more CDR region (e.g., SEQ ID NOs: 13-15, and 31-33) sequences as compared to the amino acid sequence set forth in SEQ ID NO: 1. In some instances, the light chain variable region comprises about one (e.g., 0, 1) amino acid substitution in in one or more CDR region (e.g., SEQ ID NOs: 22-24, and 40-42) sequences as compared to the amino acid sequence set forth in SEQ ID NO: 2. In some instances, the heavy chain variable region comprises at least one (e.g., 1, 2, 3, 4, 5, 6, 7, 8, 9, 10, or more) amino acid substitution in the framework region sequence as compared to the amino acid sequence set forth in SEQ ID NO: 1. In some instances, the light chain variable region comprises at least one (e.g., 1, 2, 3, 4, 5, 6, 7, 8, 9, 10, or more) amino acid substitution in the framework region sequence as compared to the amino acid sequence set forth in SEQ ID NO: 2.

In certain embodiments, the anti-CD3 scFv comprises a variable heavy chain (heavy chain variable region or VH) and a variable light chain (light chain variable region or VL) having a sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 1 and 2, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO: 1. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO: 2.

In some instances, the heavy chain variable region comprises at least one (e.g., 1, 2, 3, 4, 5, 6, 7, 8, 9, 10, or more) amino acid substitution in the sequence set forth in SEQ ID NO: 1. In certain instances, the heavy chain variable region comprises 10 or fewer amino acid (e.g., 0, 1, 2, 3, 4, 5, 6, 7, 8, 9, or 10) substitutions in the sequence set forth in SEQ ID NO: 1. In some cases, the light chain variable region comprises at least one (e.g., 1, 2, 3, 4, 5, 6, 7, 8, 9, or more) amino acid substitution in the sequence set forth in SEQ ID NO: 2. In certain cases, the light chain variable region comprises 10 or fewer amino acid (e.g., 0, 1, 2, 3, 4, 5, 6, 7, 8, 9, or 10) substitutions in the sequence set forth in SEQ ID NO: 2.

In some embodiments, the CD3 binding domain comprises a heavy chain variable region (VH) comprising a heavy chain complementary determining region 1 (HIC CDR1), a heavy chain complementary determining region 2 (HC CDR2), and a heavy chain complementary determining region 3 (1C CDR3) having an amino acid sequence as set forth in SEQ ID NO: 3. The CD3 binding domain can further comprise a light chain variable region (VL) comprising a light chain complementary determining region 1 (LC CDR1), a light chain complementary determining region 2 (LC CDR2), and a light chain complementary determining region 3 (LC CDR3) of amino acid sequence as set forth in SEQ ID NO: 4. In some embodiments, the heavy chain variable region comprises an amino acid sequence with 95-99% identity to the amino acid sequence of the heavy chain variable region as set forth in SEQ ID NO. 3 and the light chain variable region comprises an amino acid sequence with 95-99% identity to the amino acid sequence of the light chain variable region as set forth in SEQ ID NO 4.

In certain embodiments, the anti-CD3 scFv comprises a variable heavy chain (heavy chain variable region or VH) and a variable light chain (light chain variable region or VL) having an amino acid sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 3 and 4, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO:3. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO: 4. In some instances, the heavy chain variable region comprises at least one (e.g., 1, 2, 3, 4, 5, 6, 7, 8, 9, 10, or more) amino acid substitution in the sequence set forth in SEQ ID NO: 3. In certain instances, the heavy chain variable region comprises 10 or fewer amino acid (e.g., 0, 1, 2, 3, 4, 5, 6, 7, 8, 9, or 10) substitutions in the sequence set forth in SEQ ID NO:3. In some instances, the light chain variable region comprises at least one (e.g., 1, 2, 3, 4, 5, 6, 7, 8, 9, 10, or more) amino acid substitution in the sequence set forth in SEQ ID NO:4. In certain instances, the light chain variable region comprises 10 or fewer amino acid (e.g., 0, 1, 2, 3, 4, 5, 6, 7, 8, 9, or 10) substitutions in the sequence set forth in SEQ ID NO: 4. In some instances, the heavy chain variable region comprises at least one (e.g., 1, 2, 3, 4, 5, 6, 7, 8, 9, 10, or more) amino acid substitution in the framework region sequence as compared to the amino acid sequence set forth in SEQ ID NO:3. In some instances, the light chain variable region comprises at least one (e.g., 1, 2, 3, 4, 5, 6, 7, 8, 9, 10, or more) amino acid substitution in the framework region sequence as compared to the amino acid sequence set forth in SEQ ID NO: 4.

In some embodiments, the CD3 binding domain comprises a heavy chain variable region (VH comprising a heavy chain complementary determining region 1 (HC CDR1), a heavy chain complementary determining region 2 (HC CDR2), and a heavy chain complementary determining region 3 (HC CDR3) having an amino acid sequence as set forth in SEQ ID NO: 5. The CD3 binding domain can further comprise a light chain variable region (VL) comprising a light chain complementary determining region 1 (LC CDR1), a light chain complementary determining region 2 (LC CDR2), and a light chain complementary determining region 3 (LC CDR3) of amino acid sequence as set forth in SEQ ID NO: 6. In some embodiments, the heavy chain variable region comprises an amino acid sequence with 95-99% identity to the amino acid sequence of the heavy chain variable region as set forth in SEQ ID NO. 5 and the light chain variable region comprises an amino acid sequence with 95-99% identity to the amino acid sequence of the light chain variable region as set forth in SEQ ID NO 6.

In certain embodiments, the anti-CD3 scFv comprises a variable heavy chain (heavy chain variable region or VH) and a variable light chain (light chain variable region or VL) having an amino acid sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 5 and 6, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO: 5. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO: 6. In some instances, the heavy chain variable region comprises at least one (e.g., 1, 2, 3, 4, 5, 6, 7, 8, 9, 10, or more) amino acid substitution in the sequence set forth in SEQ ID NO: 5. In certain instances, the heavy chain variable region comprises 10 or fewer amino acid (e.g., 0, 1, 2, 3, 4, 5, 6, 7, 8, 9, or 10) substitutions in the sequence set forth in SEQ ID NO: 5. In some instances, the light chain variable region comprises at least one (e.g., 1, 2, 3, 4, 5, 6, 7, 8, 9, 10, or more) amino acid substitution in the sequence set forth in SEQ ID NO: 6. In certain instances, the light chain variable region comprises 10 or fewer amino acid (e.g., 0, 1, 2, 3, 4, 5, 6, 7, 8, 9, or 10) substitutions in the sequence set forth in SEQ ID NO: 6. In some instances, the heavy chain variable region comprises at least one (e.g., 1, 2, 3, 4, 5, 6, 7, 8, 9, 10, or more) amino acid substitution in the framework region sequence as compared to the amino acid sequence set forth in SEQ ID NO: 5. In some instances, the light chain variable region comprises at least one (e.g., 1, 2, 3, 4, 5, 6, 7, 8, 9, 10, or more) amino acid substitution in the framework region sequence as compared to the amino acid sequence set forth in SEQ ID NO: 6.

In some embodiments an anti-TCR antibody may be selected from the group consisting of BMA031, CIV3, and TOL101. Table 2 discloses the amino acid sequences of the VH and VL region of exemplary anti-TCR scFvs.

TABLE 2
Amino acid sequences of VH regions and VL regions of exemplary anti-TCR scFvs

| | | | SEQ |
|---|---|---|---|
| Name | Chain | Amino Acid Sequence | ID NO: |
| BMA031 | VH | EVQLQQSGPELVKPGASVKMSCKASGYKFTSYVMHWVKQKPGQ | 7 |
| (TCR) | | GLEWIGYINPYNDVTKYNEKFKGKATLTSDKSSSTAYMELSSLTSE | |
| | | DSAVHYCARGSYYDYDGFVYWGQGTLVTVSA | |
| | VL | QIVLTQSPAIMSASPGEKVTMTCSATSSVSYMHWYQQKSGTSPKR | 8 |
| | | WIYDTSKLASGVPARFSGSGSGTSYSLTISSMEAEDAATYYCQQW | |
| | | SSNPLTFGAGTKLELK | |
| CIV3 | VH | QVQLVQSGAEVKKPGSSVKVSCKASGYKFTSYVMHWVKQAPGQ | 9 |
| (TCR) | | GLEWIGYINPYNDVTKYNEKFKGKATLTADESTNTAYMELSSLRS | |
| | | EDTAVHYCARGSYYDYDGFVYWGQGTLVTVSS | |
| | VL | DIQMTQSPSTLSASVGDRVTMTCSATSSVSYMHWYQQKPGKAPK | 10 |
| | | RWIYDTSKLASGVPARFIGSGSGTEFTLTISSLQPDDFATYYCQQW | |
| | | SSNPLTFGGGTKVEIK | |
| TOL101 | VH | QVQLQQSGAELARPGASVKMSCKASGYTFTSYTMHWVKQRPGQ | 11 |
| (TCR) | | GLEWIGYINPSSGYTNYNQKFKDKATLTADKSSSTAYMQLSSLTSE | |
| | | DSAVYYCARWRDAYYAMDYWGQGTSVTVSS | |
| | VL | QIVLTQSPAIMSASPGEKVTMTCSASSSVSYMHWYQQKSGTSPKR | 12 |
| | | WIYDTSKLASGVPARFSGSGSGTSYSLTISSMEAEDAATYYCQQW | |
| | | SSNPFTFGSGTKLEIK | |

In some embodiments, the TCR binding domain comprises a heavy chain variable region (VH) comprising a heavy chain complementary, determining region 1 (HC CDR1), a heavy chain complementary determining region 2 (HC CDR2), and a heavy chain complementary determining region 3 (HC CDR3) having an amino acid sequence as set forth in SEQ ID NO: 7, 9, or 11. The TCR binding domain can further comprise a light chain variable region (VL) comprising a light chain complementary determining region 1 (LC CDR1), a light chain complementary determining region 2 (LC CDR2), and a light chain complementary determining region 3 (LC CDR3) of any anti-TCR light chain binding domain amino acid sequence as set forth in SEQ ID NO: 8, 10, or 12, wherein the VH and the NL are derived from the same antibody (BMA031, CIV3, or TOL101). In some embodiments, the heavy chain variable region comprises an amino acid sequence with 95-99% identity to the amino acid sequence of the heavy chain variable region as set forth in SEQ ID NO: 7, 9, or 11, and the light chain variable region comprises an amino acid sequence with 95-99% identity to the amino acid sequence of the light chain variable region as set forth in SEQ ID NO: 8, 10, or 12, wherein the VH and the VL are derived from the same antibody (BMA031, CIV3, or TOL101).

In certain embodiments, the anti-TCR scFv comprises a variable heavy chain (heavy chain variable region or VH) and a variable light chain (light chain variable region or VL) having an amino acid sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 7, 9, or 11 and 8, 10, or 12, respectively, wherein the VH and the VL chains are derived from the same antibody (BMA031, CIV3, or TOL101). The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO: 7, 9, or 11. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO: 8, 10, or 12. The VH and the VL can be derived from the same antibody (BMA031, CIV3, or TOL101).

In some instances, wherein the VH and the VL are derived from the same antibody (BMA031, CIV3, or TOL101), the heavy chain variable region comprises at least one (e.g., 1, 2, 3, 4, 5, 6, 7, 8, 9, 10, or more) amino acid substitution in the sequence set forth in SEQ ID NO: 7, 9, or 11. In certain instances, the heavy chain variable region comprises 10 or fewer amino acid (e.g., 0, 1, 2, 3, 4, 5, 6, 7, 8, 9, or 10) substitutions in the sequence set forth in SEQ ID NO: 7, 9, or 11. In some instances, the light chain variable region comprises at least one (e.g., 1, 2, 3, 4, 5, 6, 7, 8, 9, 10, or more) amino acid substitution in the sequence set forth in SEQ ID NO: 8, 10, or 12. In certain instances, the light chain variable region comprises 10 or fewer amino acid (e.g., 0, 1, 2, 3, 4, 5, 6, 7, 8, 9, or 10) substitutions in the sequence set forth in SEQ ID NO: 8, 10, or 12. In some instances, the heavy chain variable region comprises at least one (e.g., 1, 2, 3, 4, 5, 6, 7, 8, 9, 10, or more) amino acid substitution in the framework region sequence as compared to the amino acid sequence set forth in SEQ ID NO: 7, 9, or 11. In some instances, the light chain variable region comprises at least one (e.g., 1, 2, 3, 4, 5, 6, 7, 8, 9, 10, or more) amino acid substitution in the framework region sequence as compared to the amino acid sequence set forth in SEQ ID NO: 8, 10, or 12.

In some embodiments, the scFv of the present disclosure comprises a variable heavy chain sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to a variable heavy chain sequence of an anti-CD3 or anti-TCR antibody. In some embodiments, the scFv of the present disclosure comprises a variable light chain sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to a variable light chain sequence of an anti-CD3 or anti-TCR antibody. For instance, the anti-CD3 antibody can be any such recognized by one skilled in the art.

In some embodiments, the binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3). The amino acid sequences of the heavy chain CDR and light chain CDR regions of the above anti-CD3 and anti-TCR antibodies can be determined as described by Kabat and Chothia. In some embodiments, the binding domain comprises a HC CDR1, HC CDR2, HC CDR3, LC CDR1, LC CDR2, and LC CDR3 comprising amino acid sequences as set forth in Tables 3 to 6.

TABLE 3
Amino acid sequences of HC CDR.1-3 regions and LC CDR.1-3 regions of anti-CD3
scFvs according to the Kabat numbering scheme.

| | | | SEQ ID | | Amino Acid | SEQ ID |
|---|---|---|---|---|---|---|
| Name | Part* | Amino Acid Sequence | NO: | Part* | Sequence | NO: |
| UCHT1 | HCDR1 | GYTMN | 13 | LCDR1 | RASQDIRNYLN | 22 |
| | HCDR2 | LINPYKGVSTYNQKFKD | 14 | LCDR2 | YTSRLHS | 23 |
| | HCDR3 | SGYYGDSDWYFDV | 15 | LCDR3 | QQGNTLPWT | 24 |
| OKT-3 | HCDR1 | RYTMH | 16 | LCDR1 | SASSSVSYMN | 25 |
| | HCDR2 | YINPSRGYTNYNQKFKD | 17 | LCDR2 | DTSKLAS | 26 |
| | HCDR3 | YYDDHYCLDY | 18 | LCDR3 | QQWSSNPFT | 27 |
| 28F11 | HCDR1 | GYGMH | 19 | LCDR1 | RASQSVSSYLA | 28 |
| | HCDR2 | VIWYDGSKKYYVDSVKG | 20 | LCDR2 | DASNRAT | 29 |
| | HCDR3 | QMGYWHFDL | 21 | LCDR3 | QQRSNWPPLT | 30 |

*HCDR is an abbreviation for HC CDR; LCDR is an abbreviation for LC CDR.

TABLE 4
Amino acid sequences of HC CDR1-3 regions and LC CDR1-3 regions of anti-CD3
scFvs according to the Chothia numbering scheme

| | | | SEQ ID | | | SEQ ID |
|---|---|---|---|---|---|---|
| Name | Part* | Amino Acid Sequence | NO: | Part* | Amino Acid Sequence | NO: |
| UCHT1 | HCDR1 | GYSFTGY | 31 | LCDR1 | RASQDIRNYLN | 40 |
| | HCDR2 | NPYKGV | 32 | LCDR2 | YTSRLHS | 41 |
| | HCDR3 | SGYYGDSDWYFDV | 33 | LCDR3 | QQGNTLPWT | 42 |
| OKT-3 | HCDR1 | GYTFTRY | 34 | LCDR1 | SASSSVSYMN | 43 |
| | HCDR2 | NPSRGY | 35 | LCDR2 | DTSKLAS | 44 |
| | HCDR3 | YYDDHYCLDY | 36 | LCDR3 | QQWSSNPFT | 45 |
| 28F11 | HCDR1 | GFKFSGY | 37 | LCDR1 | RASQSVSSYLA | 46 |
| | HCDR2 | WYDGSK | 38 | LCDR2 | DASNRAT | 47 |
| | HCDR3 | QMGYWHFDL | 39 | LCDR3 | QQRSNWPPLT | 48 |

*HCDR is an abbreviation for HC CDR; LCDR is an abbreviation for LC CDR.

TABLE 5
Amino acid sequences of HC CDR1-3 regions and LC CDR1-3 regions of anti-TCR
scFvs according to the Kabat numbering scheme

| | | | SEQ ID | | Amino Acid | SEQ ID |
|---|---|---|---|---|---|---|
| Name | Part* | Amino Acid Sequence | NO: | Part* | Sequence | NO: |
| BMA031 | HCDR | SYVMH | 49 | LCDR1 | SATSSVSYMH | 58 |
| | 1 | | | | | |
| | HCDR | YINPYNDVTKYNEKFK | 50 | LCDR2 | DTSKLAS | 59 |
| | 2 | G | | | | |
| | HCDR | GSYYDYDGFVY | 51 | LCDR3 | QQWSSNPLT | 60 |
| | 3 | | | | | |
| CIV-3 | HCDR | SYVMH | 52 | LCDR1 | SATSSVSYMH | 61 |
| | 1 | | | | | |
| | HCDR | YINPYNDVTKYNEKFK | 53 | LCDR2 | DTSKLAS | 62 |
| | 2 | G | | | | |
| | HCDR | GSYYDYDGFVY | 54 | LCDR3 | QQWSSNPLT | 63 |
| | 3 | | | | | |
| TOL101 | HCDR | SYTM | 55 | LCDR1 | SASSSVSYMH | 64 |
| | 1 | | | | | |
| | HCDR | YINPSSGYTNYNQKFK | 56 | LCDR2 | DTSKLAS | 65 |
| | 2 | D | | | | |
| | HCDR | WRDAYYAMDY | 57 | LCDR3 | QQWSSNPFT | 66 |
| | 3 | | | | | |

*HCDR is an abbreviation for HC CDR; LCDR is an abbreviation for LC CDR.

TABLE 6
Amino acid sequences of HC CDR1-3 regions and LC CDR 1-3 regions of anti-TCR
scFvs according to the Chothia numbering scheme

| | | Amino Acid | SEQ ID | | Amino Acid Sequence | SEQ ID |
|---|---|---|---|---|---|---|
| Name | Part* | Sequence | NO: | Part* | | NO: |
| BMA031 | HCDR1 | GYKFTSY | 67 | LCDR1 | SATSSVSYMH | 76 |
| | HCDR2 | NPYNDV | 68 | LCDR2 | DTSKLAS | 77 |
| | HCDR3 | GSYYDYDGFVY | 69 | LCDR3 | QQWSSNPLT | 78 |
| CIV-3 | HCDR1 | GYKFTSY | 70 | LCDR1 | SATSSVSYMH | 79 |
| | HCDR2 | NPYNDV | 71 | LCDR2 | DTSKLAS | 80 |
| | HCDR3 | GSYYDYDGFVY | 72 | LCDR3 | QQWSSNPLT | 81 |
| TOL101 | HCDR1 | GYTFTSY | 73 | LCDR1 | SASSSVSYMH | 82 |
| | HCDR2 | NPSSGY | 74 | LCDR2 | DTSKLAS | 83 |
| | HCDR3 | WRDAYYAMDY | 75 | LCDR3 | QQWSSNPFT | 84 |

*HCDR is an abbreviation for HC CDR; LCDR is an abbreviation for LC CDR.

In some embodiments, the HC CDR1, HC CDR2, HC CDR3, LC CDR1, LC CDR2, and LC CDR3 comprise the amino acid sequences of: (i) SEQ ID NOs: 13-15, and 22-24, respectively; or (ii) SEQ ID NOs: 31-33, and 40-42 respectively.

In some embodiments, the HC CDR1, HC CDR2, HC CDR3, LC CDR1, LC CDHR2, and LC CDR3 comprise the amino acid sequences of: (i) SEQ ID NOs: 16-45 and 25-27, respectively; or (ii) SEQ ID NOs: 34-36, and 43-45 respectively.

In some embodiments, the HC CDR1, HC CDR2, HC CDR3, LC CDR1, LC CDR2, and LC CDR3 comprise the amino acid sequences of: (i) SEQ ID NOs: 19-21, and 28-30, respectively: or (ii) SEQ ID NOs: 37-3% and 6-48 respectively.

In some embodiments, the HC CDR1, HC CDR2, HC CDR3, LC CDR1, LC CDR2, and LC CDR3 comprise the amino acid sequences of: (i) SEQ ID NOs: 49-51, and 58-60, respectively; or (ii) SEQ ID NOs: 67-69, and 76-78 respectively.

In some embodiments, the HC CDR1, HC CDR2, HC CDR3, LC CDR1, LC CDR2, and LC CDR3 comprise the amino acid sequences of: (i) SEQ ID NOs: 52-54, and 61-63, respectively; or (ii) SEQ ID NOs 70-72, and 79-81 respectively.

In some embodiments, the HC CDR1, HC CDR2, HC CDR3, LC CDR1, LC CDR2, and LC CDR3 comprise the amino acid sequences of: (i) SEQ ID NOs: 55-57, and 64-66, respectively; or (ii) SEQ ID NOs: 73-75, and 82-84 respectively.

In certain embodiments, the CD3 binding domain molecule described herein includes: (1) one, two, or three heavy chain (HC) CDRs chosen from one of the following: (i) a HC CDR1 of SEQ ID NO: 13, HC CDR2 of SEQ ID NO: 14 and HC CDR3 of SEQ ID NO: 15; or (ii) a HC CDR1 of SEQ ID NO: 31, HC CDR2 of SEQ ID NO: 32 and HC CDR3 of SEQ ID NO: 33; and/or (2) one, two, or three light chain (LC) CDRs chosen from one of the following: (i) a LC CDR1 of SEQ ID NO: 22, LC CDR2 of SEQ ID NO: 23 and LC CDR3 of SEQ ID NO: 24; or (ii) a LC CDR1 of SEQ ID NO: 40, LC CDR2 of SEQ ID NO: 41 and LC CDR3 of SEQ ID NO: 42.

In various cases described herein, a binding domain may comprise a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a sequence of a reference binding domain. And when the binding domain is an antibody or fragment thereof (e.g., a functional fragment or the fragment having a variable domain), the binding domain can comprise the heavy chain and/or light chain CDR regions of the sequence of the reference binding domain and having sequence variations in the framework regions or other regions outside of the CDR regions.

In some embodiments, the CD3 binding domain molecule described herein includes: (1) one, two, or three heavy chain (HC) CDRs chosen from one of the following: (i) a HC CDR1 of SEQ ID NO: 19, HC CDR2 of SEQ ID NO: 20 and HC CDR3 of SEQ ID NO: 21; or (ii) a HC CDR1 of SEQ ID NO: 37, HC CDR2 of SEQ ID NO: 38 and HC CDR3 of SEQ ID NO: 39; and/or (2) one, two, or three light chain (LC) CDRs chosen from one of the following: (i) a LC CDR1 of SEQ ID NO: 28, LC CDR2 of SEQ ID NO: 29 and LC CDR3 of SEQ ID NO: 30; or (ii) a LC CDR1 of SEQ ID NO: 46, LC CDR2 of SEQ ID NO: 47 and LC CDR3 of SEQ ID NO: 48.

In some embodiments, the CD3 binding molecule described herein includes: (i) a HC CDR1 comprising a sequence having at least 90%, at least 91%, at least 92%, at least 93%, at least 94%, at least 95%, at least 96%, at least 97%, at least 98%, at least 99%, or 100% sequence identity to an amino acid sequence as set forth in SEQ ID NOs: 19 or 37; (ii) a HC CDR2 comprising a sequence having at least 90%, at least 91%, at least 92%, at least 93%, at least 94%, at least 95%, at least 96%, at least 97%, at least 98%, at least 99%, or 100% sequence identity to an amino acid sequence as set forth in SEQ ID NOs: 20 or 38; (iii) a HC CDR3 comprising a sequence having at least 90%, at least 91%, at least 92%, at least 93%, at least 94%, at least 95%, at least 96%, at least 97%, at least 98%, at least 99%, or 100% sequence identity to an amino acid sequence as set forth in SEQ ID NOs: 21 or 39; (iv) a LC CDR1 comprising a sequence having at least 90%, at least 91%, at least 92%, at least 93%, at least 94%, at least 95%, at least 96%, at least 97%, at least 98%, at least 99%, or 100% sequence identity to an amino acid sequence as set forth in SEQ ID NOs: 28 or 46; (v) a LC CDR2 comprising a sequence having at least 90%, at least 91%, at least 92%, at least 93%, at least 94%, at least 95%, at least 96%, at least 97%, at least 98%, at least 99%, or 100% sequence identity to an amino acid sequence as set forth in SEQ ID NOs: 29 or 47; and/or (vi) a LC CDR3 comprising a sequence having at least 90%, at least 91%, at least 92%, at least 93%, at least 94%, at least 95%, at least 96%, at least 97%, at least 98%, at least 99%, or 100% sequence identity to an amino acid sequence as set forth in SEQ ID NOs: 30 or 48.

In some embodiments, the binding domain includes a Gly-Ser linker, e.g., a linker having an amino acid sequence GGGGSGGGGSGGGGSGGGGS as set forth in SEQ ID NO: 85. The light chain variable region and heavy chain variable region of a scFv can be, e.g., in any of the following orientations: light chain variable region-linker-heavy chain variable region or heavy chain variable region-linker-light chain variable region. In some embodiments, the linker may comprise an amino acid sequence (GGGGS)n where n can range from 1 to 6, e.g., 1, 2, 3, 4, 5, or 6 (SEQ ID NO: 86).

Non-limiting examples of a linker include (GS)n (SEQ ID NO: 87), (GGS)n (SEQ ID NO: 88), (Gly3Ser)n (SEQ ID NO: 89), (Gly2SerGly)n (SEQ ID NO: 90), (Gly2SerGly2)n (SEQ ID NO: 91), or (Gly4Ser)n (SEQ ID NO: 92), wherein n is 1, 2, 3, 4, 5, 6, 7, 8, 9, or 10. In some embodiments, the linker is (GGGGS)n where n can range from 1 to 6, e.g., 1, 2, 3, 4, 5, or 6 (SEQ ID NO: 86). In some embodiment, the linker is (Gly4Ser)3 (SEQ ID NO: 93) or (Gly4Ser)4 (SEQ ID NO: 85). In some embodiments, the linker may comprise an amino acid sequence of GGGGS GGGGS GGGGS (SEQ ID NO: 93). In some embodiments, the linker may comprise an amino acid sequence of GGGGSGGGGS (SEQ ID NO: 94).

Variation in the linker length may retain or enhance activity, giving rise to superior efficacy in activity studies. The light chain variable region and heavy chain variable region of a scFv can be, e.g., in any of the following orientations: light chain variable region-linker-heavy chain variable region or heavy chain variable region-linker-light chain variable region. In various embodiments, peptide linkers having lengths of about 5 to about 100 amino acids, inclusive, can be used in the present invention. In certain embodiments, peptide linkers having lengths of about 20 to about 40 amino acids, inclusive, can be used in the present invention. In some embodiments, peptide linkers having lengths of at least 5 amino acids, at least 10 amino acids, at least 15 amino acids, at least 20 amino acids, at least 25 amino acids, at least 30 amino acids, at least 35 amino acids, or at least 40 amino acids can be used in the present disclosure.

In some embodiments, the CD3 CAR further comprises a hinge and transmembrane sequence. The hinge and transmembrane sequences suitable for use in the present disclosure are known in the art, and provided in, e.g., publication WO2016/126213, incorporated by reference in its entirety. In some embodiments, the isolated CAR molecule comprises a hinge and a transmembrane domain of a protein selected from the group consisting of the alpha or beta chain of the T-cell receptor, CD28, CD3ε, CD3δ, CD3γ, CD8, CD45, CD4, CD5, CD9, CD16, CD22, CD33, CD37, CD64, CD30, CD36, CD134, CD137 and CD154. In some embodiments, the hinge and the transmembrane domain comprise an amino acid sequence as set forth in SEQ ID NO: 101. In some embodiments, the hinge and the transmembrane domain comprise an amino acid sequence having at least one, two or three modifications (e.g., substitutions) but not more than 20, 10 or 5 modifications (e.g., substitutions) of an amino acid sequence of SEQ ID NO: 101, or a sequence with 95-99% identity to an amino acid sequence of SEQ ID NO: 101. In some embodiments, the hinge comprises an amino acid sequence as set forth in SEQ ID NO: 99. In some embodiments, the hinge comprises an amino acid sequence having at least one, two or three modifications (e.g., substitutions) but not more than 20, 10 or 5 modifications (e.g., substitutions) of an amino acid sequence of SEQ ID NO: 99, or a sequence with 95-99% identity to an amino acid sequence of SEQ ID NO: 99. In some embodiments, the transmembrane domain comprises an amino acid sequence as set forth in SEQ ID NO: 100. In some embodiments, the transmembrane domain comprises an amino acid sequence having at least one, two or three modifications (e.g., substitutions) but not more than 20, 10 or 5 modifications (e.g., substitutions) of an amino acid sequence of SEQ ID NO: 100, or a sequence with 95-99% identity to an amino acid sequence of SEQ ID NO: 100.

In some embodiments, the hinge and transmembrane domain of the CD3 CAR can include a domain (e.g., transmembrane domain) from 4-1BB, CD28, CD34, CD4, CD8, FcεRIγ, CD16, OX40, CD3ζ, CD3ε, CD3γ, CD3δ, TCRα, TCRβ, CD32, CD64, VEGFR2, FAS, FGFR2B, or another transmembrane protein.

In some embodiments, the isolated CAR molecule further comprises a sequence encoding an intracellular signaling domain, e.g., an intracellular signaling domain described herein, e.g., an intracellular signaling domain comprising a costimulatory domain and a cytoplasmic signaling domain. In some embodiments, the intracellular signaling domain comprises a functional signaling domain of a protein selected from the group consisting of OX40, CD2, CD27, CD28, CD5, CD3, e.g., CD3ζ, CD3γ, CD3δ, CD3ε, ICAM-1, LFA-1 (CDlla/CD18), 4-1BB (CD137), CD30, CD84, CRTAM, DR3, GITR, HVEM, ICOS, SLAMFI, TIM1, CD79a, CD79b, DAP12 and FCER1G. In some embodiments, the intracellular signaling domain comprises an amino acid sequence of SEQ ID NO: 102 and/or SEQ ID NO: 103. In some embodiments, the intracellular signaling domain comprises an amino acid sequence having at least one, two or three modifications (e.g., substitutions) but not more than 20, 10 or 5 modifications (e.g., substitutions) of an amino acid sequence of SEQ ID NO: 102 and/or 103, or a sequence with 95-99% identity to an amino acid sequence of SEQ ID NO: 102 and/or 103. In one embodiment, the intracellular signaling domain comprises the sequence of SEQ ID NO:102 and the sequence of SEQ ID NO: 103, wherein the sequences comprising the intracellular signaling domain are expressed in the same frame and as a single polypeptide chain.

In some embodiments, the costimulatory domain of the CAR can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to an intracellular signaling domain of CD28, OX40, ICOS, CD27, GITR, HVEM, TIM1, LFA1, CD2, 4-1BB, CD30, CD84, CRTAM, DR3, ICOS, or SLAMFI. The protein sequence of the intracellular signaling domain of each of these proteins can be found on public database such as UniProt.

In some embodiments, the costimulatory domain comprises a functional signaling domain of a protein selected from the group consisting of OX40, CD2, CD27, CD28, CD5, CD3, e.g., CD3ζ, CD3γ, CD3δ, CD3ε, ICAM-1, LFA-1 (CDlla/CD18) and 4-1BB (CD137). In some embodiments, the costimulatory domain comprises an amino acid sequence of SEQ ID NO: 102. In some embodiments, the costimulatory domain comprises an amino acid sequence having at least one, two or three modifications (e.g., substitutions) but not more than 20, 10 or 5 modifications (e.g., substitutions) of an amino acid sequence of SEQ ID NO: 102, or a sequence with 95-99% identity to an amino acid sequence of SEQ ID NO: 102. In some embodiments, the costimulatory domain of the CAR can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2. In some embodiments, the costimulatory domain 4-1BB can be replaced by another costimulatory domain from a costimulatory molecule such as CD28, OX40, ICOS, CD27, GITR, HVEM, TIM1, LFA1, or CD2. In some embodiments, the costimulatory domain of 4-1BB can also include another costimulatory domain (or a portion thereof) from a costimulatory molecule such as CD28, OX40, ICOS, CD27, GITR, HVEM, TIM1, LFA1, or CD2. In some embodiments, the additional costimulatory domain can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2. In other embodiments, the additional costimulatory domain comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one or more intracellular signaling domain fragment(s) of CD28, OX40, ICOS, CD27, GITR, HVEM, TIM1, LFA1, or CD2.

In some embodiments, the intracellular signaling domain comprises a cytoplasmic signaling domain, e.g., CD3ζ. In some embodiments, the cytoplasmic signaling domain comprises the sequence of SEQ ID NO: 103. In one embodiment, the intracellular signaling domain comprises an amino acid sequence having at least one, two or three modifications (e.g., substitutions) but not more than 20, 10 or 5 modifications (e.g., substitutions) of an amino acid sequence of SEQ ID NO: 103 or a sequence with 95-99% identity to an amino acid sequence of SEQ ID NO: 105 and/or an amino acid sequence of SEQ ID NO: 103.

In some instances, the intracellular signaling domain comprises an immunoreceptor tyrosine-based activation motif (ITAM) or a portion thereof, as long as it possesses the desired function. The intracellular signaling domain of the CAR can include a sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to an ITAM. In certain embodiments, the intracellular signaling domain can have at least 95% sequence identity, at least 96% sequence identity, at least 97% sequence identity, at least 98% sequence identity, at least 99% sequence identity, or 100% sequence identity to an ITAM of CD3ε, CD3γ, CD3δ, or CD3ζ, as long as it possesses the desired function.

In some embodiment, the isolated CAR molecule further comprises a leader sequence, e.g., a leader sequence described herein. In some embodiments, the leader sequence encodes a CD8a signal peptide. In some embodiments, the leader sequence comprises an amino acid sequence of SEQ ID NO: 97, or a sequence with 95-99% identity to an amino acid sequence of SEQ ID NO: 97.

In one aspect, the present disclosure relates to an engineered immune cell comprising a nucleic acid that comprises a nucleotide sequence encoding a chimeric antigen receptor (CAR), comprising an antigen binding domain, e.g., a CD3 antigen binding domain and one or more intracellular signaling domains comprising a costimulatory domain and a cytoplasmic signaling domain selected from 4-1BB and CD3 respectively. In some embodiments, the antigen binding domain specifically binds the epsilon chain of CD3. The CAR of the present disclosure is sometimes referred to herein as “anti-CD3-41BB-CD3c” (CD3 CAR) or “anti-TCR-41BB-CD3ζ” (TCR CAR). In some embodiments, the CAR comprises an amino acid sequence as set forth in any of SEQ ID NOs: 95-96, 120-121, 104-105, 107-108, 110-111, 125-126, or 128-131. In some embodiments, the CAR comprises an amino acid sequence having at least one, two or three modifications but not more than 30, 20 or 10 modifications of the amino acid sequence as set forth in any of SEQ ID NOs: 95-96, 120-121, 104-105, 107-108, 110-111, 125-126, or 128-131. In some embodiments, the CD3 CAR comprises an amino acid sequence with 95-99% identity to the amino acid sequence as set forth in any of SEQ ID NOs: 95-96, 120-121, 104-105, 107-108, 110-111, 125-126, or 128-131.

In some embodiments, an “engineered” immune cell includes an immune cell that has been genetically modified as compared to a naturally-occurring immune cell. In some embodiments, an engineered T cell produced according to the present methods carries a nucleic acid comprising a nucleotide sequence, encoding a polypeptide as set forth in any of SEQ ID NOs: 95-96, 120-121, 104-105, 107-108, 110-111, 125-126, or 128-131 that does not naturally occur in a T cell from which it was derived.

In some embodiments, the present disclosure relates to an engineered immune cell comprising a nucleic acid that comprises a nucleotide sequence encoding a chimeric antigen receptor (CAR), comprising an antigen binding domain, e.g., a CD3 or TCR antigen binding domain and one or more intracellular signaling domains comprising a costimulatory domain and a cytoplasmic signaling domain selected from 4-1BB and CD3ζ respectively. In some embodiments, the antigen binding domain specifically binds the epsilon chain of CD3. The CAR of the present disclosure is sometimes referred to herein as an “anti-CD3-41BB-CD3c” (CD3 CAR) or an “anti-TCR-41BB-CD3ζ” (TCR CAR). In some embodiments, the CD3 CAR comprises an amino acid sequence as set forth in SEQ ID NOs:120 or 121. In some embodiments, the CD3 CAR comprises an amino acid sequence having at least one, two or three modifications but not more than 30, 20 or 10 modifications of the amino acid sequence as set forth in SEQ ID NOs:120 or 121. In some embodiments, the CD3 CAR comprises an amino acid sequence with 95-99% identity to the amino acid sequence as set forth in SEQ ID NOs:120 or 121.

In some embodiments, an “engineered” immune cell includes an immune cell that has been genetically modified as compared to a naturally-occurring immune cell. In some embodiments, an engineered T cell produced according to the present methods carries a nucleic acid comprising a nucleotide sequence, encoding a polypeptide as set forth in any of SEQ ID NOs: 95-96, 120-121, 104-105, 107-108, 110-111, 125-126, or 128-131 that does not naturally occur in a T cell from which it was derived. In one aspect, the disclosure provides an isolated CAR molecule comprising a leader sequence, e.g., a leader sequence described herein, e.g., a leader sequence of SEQ ID NO: 97, or having 95-99% identity thereof, a binding domain described herein, e.g., a binding domain comprising an HC and a LC described in Tables 1-2 or a HC CDR1, a HC CDR2, a HC CDR3, a LC CDR1, a LC CDR2 and a LC CDR3 described in Tables 3-6, or a sequence with 95-99% identify to sequences disclosed in Tables 1-6; a transmembrane and a hinge region, e.g., a transmembrane and a hinge region described herein, e.g., a transmembrane and a hinge region of SEQ ID NO:101 or having 95-99% identity thereof (e.g., a hinge region of SEQ ID NO:99 or having 95-99% identity thereof and a transmembrane region of SEQ ID NO:100 or having 95-99% identity thereof); an intracellular signaling domain, e.g., an intracellular signaling domain described herein; an intracellular signaling region comprising a costimulatory domain; e.g., a costimulatory domain comprising a SEQ ID NO: 102 or having 95-99% identity thereof and/or a cytoplasmic signaling domain (e.g., a cytoplasmic signaling domain comprising a SEQ ID NO: 103 or having 95-99% identity thereof). In one embodiment, the intracellular signaling domain comprises, a 4-IBB domain having a sequence of SEQ ID NO:102, or having 95-99% identity thereof, and/or a cytoplasmic signaling domain, e.g., a cytoplasmic signaling domain described herein, e.g., a CD3 zeta stimulatory domain having a sequence of SEQ ID NO:103 or having 95-99% identity thereof.

In one aspect, the disclosure provides a nucleotide encoding an isolated CAR molecule comprising a leader sequence, e.g., a leader sequence described herein, e.g., a leader sequence of SEQ ID NO: 97, or having 95-99% identity thereof, a binding domain described herein, e.g., binding domain comprising a HC CDR1, a HC CDR2, a HC CDR3, a LC CDR1, a LC CDR2 and a LC CDR3 described herein, e.g., an anti-CD3 or anti-TCR binding domain comprising an HC and a LC described in Tables 1-2, or a HC CDR1, a HC CDR2, a HC CDR3, a LC CDR1, a LC CDR2 and a LC CDR3 described Tables 3-6, or a sequence with 95-99% identify to sequences disclosed in Tables 1-6; a transmembrane and a hinge region, e.g., a transmembrane and a hinge region described herein, e.g., a transmembrane and a hinge region of SEQ ID NO:101 or having 95-99% identity thereof (e.g., a hinge region of SEQ ID NO:99 or having 95-99% identity thereof and a transmembrane region of SEQ ID NO:100 or having 95-99% identity thereof); an intracellular signaling domain, e.g., an intracellular signaling domain described herein; an intracellular signaling region comprising a costimulatory domain; e.g., a costimulatory domain comprising a SEQ ID NO: 102 or having 95-99% identity thereof and/or a cytoplasmic signaling domain (e.g., a cytoplasmic signaling domain comprising a SEQ ID NO: 103 or having 95-99% identity thereof). In one embodiment, the intracellular signaling domain comprises, a 4-IBB domain having a sequence of SEQ ID NO:102, or having 95-99% identity thereof, and/or a cytoplasmic signaling domain, e.g., a cytoplasmic signaling domain described herein, e.g., a CD3 zeta stimulatory domain having a sequence of SEQ ID NO:103 or having 95-99% identity thereof.

TABLE 7
Amino acid sequences of exemplary CD3 CAR polypeptides and select components

| | | | SEQ |
|---|---|---|---|
| | | | ID |
| | Description | Amino Acid Sequence | NO: |
| Anti- | CD3 CAR | MALPVTALLLPLALLLHAARPDIQMTQTTSSLSASLGDRVTISCRASQ | 95 |
| CD3 | | DIRNYLNWYQQKPDGTVKLLIYYTSRLHSGVPSKFSGSGSGTDYSLTI | |
| (UCHT1) | | SNLEQEDIATYFCQQGNTLPWTFAGGTKLEIKGGGGSGGGGSGGGGS | |
| | | GGGGSEVQLQQSGPELVKPGASMKISCKASGYSFTGYTMNWVKQSH | |
| | | GKNLEWMGLINPYKGVSTYNQKFKDKATLTVDKSSSTAYMELLSLTS | |
| | | EDSAVYYCARSGYYGDSDWYFDVWGQGTTLTVFSKPTTTPAPRPPTP | |
| | | APTIASQPLSLRPEACRPAAGGAVHTRGLDFACDIYIWAPLAGTCGVL | |
| | | LLSLVITLYCKRGRKKLLYIFKQPFMRPVQTTQEEDGCCRFPEEEEGG | |
| | | CELRVKFSRSADAPAYQQGQNQLYNELNLGRREEYDVLDKRRGRDP | |
| | | EMGGKPRRKNPQEGLYNELQKDKMAEAYSEIGMKGERRRGKGHDG | |
| | | LYQGLSTATKDTYDALHMQALPPR | |
| | CD3 CAR | DIQMTQTTSSLSASLGDRVTISCRASQDIRNYLNWYQQKPDGTVKLLI | 96 |
| | without | YYTSRLHSGVPSKFSGSGSGTDYSLTISNLEQEDIATYFCQQGNTLPWT | |
| | signal | FAGGTKLEIKGGGGSGGGGSGGGGSGGGGSEVQLQQSGPELVKPGAS | |
| | peptide | MKISCKASGYSFTGYTMNWVKQSHGKNLEWMGLINPYKGVSTYNQ | |
| | | KFKDKATLTVDKSSSTAYMELLSLTSEDSAVYYCARSGYYGDSDWYF | |
| | | DVWGQGTTLTVFSKPTTTPAPRPPTPAPTIASQPLSLRPEACRPAAGGA | |
| | | VHTRGLDFACDIYIWAPLAGTCGVLLLSLVITLYCKRGRKKLLYIFKQ | |
| | | PFMRPVQTTQEEDGCSCRFPEEEEGGCELRVKFSRSADAPAYQQGQN | |
| | | QLYNELNLGRREEYDVLDKRRGRDPEMGGKPRRKNPQEGLYNELQK | |
| | | DKMAEAYSEIGMKGERRRGKGHDGLYQGLSTATKDTYDALHMQAL | |
| | | PPR | |
| CAR | CD8α | MALPVTALLLPLALLLHAARP | 97 |
| | signal | | |
| | peptide | | |
| | scFv | DIQMTQTTSSLSASLGDRVTISCRASQDIRNYLNWYQQKPDGTVKLLI | 98 |
| | | YYTSRLHSGVPSKFSGSGSGTDYSLTISNLEQEDIATYFCQQGNTLPWT | |
| | | FAGGTKLEIKGGGGSGGGGSGGGGSGGGGSEVQLQQSGPELVKPGAS | |
| | | MKISCKASGYSFTGYTMNWVKQSHGKNLEWMGLINPYKGVSTYNQ | |
| | | KFKDKATLTVDKSSSTAYMELLSLTSEDSAVYYCARSGYYGDSDWYF | |
| | | DVWGQGTTLTVFS | |
| | VH | EVQLQQSGPELVKPGASMKISCKASGYSFTGYTMNWVKQSHGKNLE | 1 |
| | | WMGLINPYKGVSTYNQKFKDKATLTVDKSSSTAYMELLSLTSEDSAV | |
| | | YYCARSGYYGDSDWYFDVWGQGTTLTVFS | |
| | VL | DIQMTQTTSSLSASLGDRVTISCRASQDIRNYLNWYQQKPDGTVKLLI | 2 |
| | | YYTSRLHSGVPSKFSGSGSGTDYSLTISNLEQEDIATYFCQQGNTLPWT | |
| | | FAGGTKLEIK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACDIYI | 101 |
| | hinge and | WAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPEMG | 103 |
| | signaling | GKPRRKNPQEGLYNELQKDKMAEAYSEIGMKGERRRGKGHDGLYQ | |
| | domain of | GLSTATKDTYDALHMQALPPR | |
| | CD3ζ | | |
| Anti- | CD3 CAR | MALPVTALLLPLALLLHAARPQIVLTQSPAIMSASPGEKVTMTCSASSS | 104 |
| CD3 | | VSYMNWYQQKSGTSPKRWIYDTSKLASGVPAHFRGSGSGTSYSLTIS | |
| (OKT3) | | GMEAEDAATYYCQQWSSNPFTFGSGTKLEINRGGGGSGGGGSGGGG | |
| CAR | | SGGGGSEVQLQQSGAELARPGASVKMSCKASGYTFTRYTMHWVKQR | |
| | | PGQGLEWIGYINPSRGYTNYNQKFKDKATLTTDKSSSTAYMQLSSLTS | |
| | | EDSAVYYCARYYDDHYCLDYWGQGTTLTVSSAKPTTTPAPRPPTPAP | |
| | | TIASQPLSLRPEACRPAAGGAVHTRGLDFACDIYIWAPLAGTCGVLLL | |
| | | SLVITLYCKRGRKKLLYIFKQPFMRPVQTTQEEDGCSCRFPEEEEGGCE | |
| | | LRVKFSRSADAPAYQQGQNQLYNELNLGRREEYDVLDKRRGRDPEM | |
| | | GGKPRRKNPQEGLYNELQKDKMAEAYSEIGMKGERRRGKGHDGLY | |
| | | QGLSTATKDTYDALHMQALPPR | |
| | CD3 CAR | QIVLTQSPAIMSASPGEKVTMTCSASSSVSYMNWYQQKSGTSPKRWI | 105 |
| | without | YDTSKLASGVPAHFRGSGSGTSYSLTISGMEAEDAATYYCQQWSSNP | |
| | signal | FTFGSGTKLEINRGGGGSGGGGSGGGGSGGGGSEVQLQQSGAELARP | |
| | peptide | GASVKMSCKASGYTFTRYTMHWVKQRPGQGLEWIGYINPSRGYTNY | |
| | | NQKFKDKATLTTDKSSSTAYMQLSSLTSEDSAVYYCARYYDDHYCL | |
| | | DYWGQGTTLTVSSAKPTTTPAPRPPTPAPTIASQPLSLRPEACRPAAGG | |
| | | AVHTRGLDFACDIYIWAPLAGTCGVLLLSLVITLYCKRGRKKLLYIFK | |
| | | QPFMRPVQTTQEEDGCSCRFPEEEEGGCELRVKFSRSADAPAYQQGQ | |
| | | NQLYNELNLGRREEYDVLDKRRGRDPEMGGKPRRKNPQEGLYNELQ | |
| | | KDKMAEAYSEIGMKGERRRGKGHDGLYQGLSTATKDTYDALHMQA | |
| | | LPPR | |
| | CD8α | MALPVTALLLPLALLLHAARP | 97 |
| | signal | | |
| | peptide | | |
| | scFv | QIVLTQSPAIMSASPGEKVTMTCSASSSVSYMNWYQQKSGTSPKRWI | 106 |
| | | YDTSKLASGVPAHFRGSGSGTSYSLTISGMEAEDAATYYCQQWSSNP | |
| | | FTFGSGTKLEINRGGGGSGGGGSGGGGSGGGGSEVQLQQSGAELARP | |
| | | GASVKMSCKASGYTFTRYTMHWVKQRPGQGLEWIGYINPSRGYTNY | |
| | | NQKFKDKATLTTDKSSSTAYMQLSSLTSEDSAVYYCARYYDDHYCL | |
| | | DYWGQGTTLTVSSA | |
| | VH | EVQLQQSGAELARPGASVKMSCKASGYTFTRYTMHWVKQRPGQGLE | 3 |
| | | WIGYINPSRGYTNYNQKFKDKATLTTDKSSSTAYMQLSSLTSEDSAVY | |
| | | YCARYYDDHYCLDYWGQGTTLTVSSA | |
| | VL | QIVLTQSPAIMSASPGEKVTMTCSASSSVSYMNWYQQKSGTSPKRWI | 4 |
| | | YDTSKLASGVPAHFRGSGSGTSYSLTISGMEAEDAATYYCQQWSSNP | |
| | | FTFGSGTKLEINR | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACDIYI | 101 |
| | hinge and | WAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPEMG | 103 |
| | signaling | GKPRRKNPQEGLYNELQKDKMAEAYSEIGMKGERRRGKGHDGLYQ | |
| | CD3ζ | GLSTATKDTYDALHMQALPPR | |
| Anti- | CD3 CAR | MALPVTALLLPLALLLHAARPEIVLTQSPATLSLSPGERATLSCRASQS | 107 |
| CD3 | | VSSYLAWYQQKPGQAPRLLIYDASNRATGIPARFSGSGSGTDFTLTISS | |
| (28F11) | | LEPEDFAVYYCQQRSNWPPLTFGGGTKVEIKGGGGSGGGGSGGGGSG | |
| CAR | | GGGSQVQLVESGGGVVQPGRSLRLSCAASGFKFSGYGMHWVRQAPG | |
| | | KGLEWVAVIWYDGSKKYYVDSVKGRFTISRDNSKNTLYLQMNSLRA | |
| | | EDTAVYYCARQMGYWHFDLWGRGTLVTVSSKPTTTPAPRPPTPAPTI | |
| | | ASQPLSLRPEACRPAAGGAVHTRGLDFACDIYIWAPLAGTCGVLLLSL | |
| | | VITLYCKRGRKKLLYIFKQPFMRPVQTTQEEDGCCRFPEEEEGGCEL | |
| | | RVKFSRSADAPAYQQGQNQLYNELNLGRREEYDVLDKRRGRDPEMG | |
| | | GKPRRKNPQEGLYNELQKDKMAEAYSEIGMKGERRRGKGHDGLYQ | |
| | | GLSTATKDTYDALHMQALPPR | |
| | CD3 CAR | EIVLTQSPATLSLSPGERATLSCRASQSVSSYLAWYQQKPGQAPRLLIY | 108 |
| | without | DASNRATGIPARFSGSGSGTDFTLTISSLEPEDFAVYYCQQRSNWPPLT | |
| | signal | FGGGTKVEIKGGGGSGGGGSGGGGSGGGGSQVQLVESGGGVVQPGR | |
| | peptide | SLRLSCAASGFKFSGYGMHWVRQAPGKGLEWVAVIWYDGSKKYYV | |
| | | DSVKGRFTISRDNSKNTLYLQMNSLRAEDTAVYYCARQMGYWHFDL | |
| | | WGRGTLVTVSSKPTTTPAPRPPTPAPTIASQPLSLRPEACRPAAGGAVH | |
| | | TRGLDFACDIYIWAPLAGTCGVLLLSLVITLYCKRGRKKLLYIFKQPF | |
| | | MRPVQTTQEEDGCCRFPEEEEGGCELRVKFSRSADAPAYQQGQNQL | |
| | | YNELNLGRREEYDVLDKRRGRDPEMGGKPRRKNPQEGLYNELQKDK | |
| | | MAEAYSEIGMKGERRRGKGHDGLYQGLSTATKDTYDALHMQALPPR | |
| | CD8α | MALPVTALLLPLALLLHAARP | 97 |
| | signal | | |
| | peptide | | |
| | scFv | EIVLTQSPATLSLSPGERATLSCRASQSVSSYLAWYQQKPGQAPRLLIY | 109 |
| | | DASNRATGIPARFSGSGSGTDFTLTISSLEPEDFAVYYCQQRSNWPPLT | |
| | | FGGGTKVEIKGGGGSGGGGSGGGGSGGGGSQVQLVESGGGVVQPGR | |
| | | SLRLSCAASGFKFSGYGMHWVRQAPGKGLEWVAVIWYDGSKKYYV | |
| | | DSVKGRFTISRDNSKNTLYLQMNSLRAEDTAVYYCARQMGYWHFDL | |
| | | WGRGTLVTVSS | |
| | VH | QVQLVESGGGVVQPGRSLRLSCAASGFKFSGYGMHWVRQAPGKGLE | 5 |
| | | WVAVIWYDGSKKYYVDSVKGRFTISRDNSKNTLYLQMNSLRAEDTA | |
| | | VYYCARQMGYWHFDLWGRGTLVTVSS | |
| | VL | EIVLTQSPATLSLSPGERATLSCRASQSVSSYLAWYQQKPGQAPRLLIY | 6 |
| | | DASNRATGIPARFSGSGSGTDFTLTISSLEPEDFAVYYCQQRSNWPPLT | |
| | | FGGGTKVEIK | |
| | scFv | GGGGSGGGGSGGGGGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACDIYI | 101 |
| | hinge and | WAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPEMG | 103 |
| | signaling | GKPRRKNPQEGLYNELQKDKMAEAYSEIGMKGERRRGKGHDGLYQ | |
| | domain of | GLSTATKDTYDALHMQALPPR | |
| | CD3ζ | | |

In some embodiments, the disclosure provides a cell (e.g., an immune effector cell, e.g., T cell or NK cell) engineered to express a CAR, comprising an antigen binding domain again a TCR receptor, e.g., BMA031 CAR, CIV-3 CAR or TOL101-CAR, wherein the CAR T cell (“CART”) or CAR NK (“CAR-NK”) cell can target a cell expressing the TCR, e.g. a T cell, T cell lymphoma, or T cell leukemia. In one aspect, a cell is transformed with the CAR, e.g., CIV-3 CAR or TOL101-CAR and the CAR, e.g., CIV-3 CAR or TOL101-CAR is expressed on the cell surface. In one aspect, the disclosure provides a polypeptide construct containing a target-binding molecule that binds a target (e.g, TCR) to be removed or neutralized. In one aspect, the disclosure provides a method to treat a disease, e.g., cancer in a subject in need thereof by administering a composition comprising cells expressing TCR chimeric antigen receptors (TCR), e.g., CIV-3 CAR or TOL101-CAR, optionally in combination with a second agent that downregulates TCR expression on the effector T cells. Table 8 provides amino acid sequences of some exemplary anti-TCR CAR and selected components.

TABLE 8
Amino acid sequences of exemplary anti-TCR CAR and selected components

| | | | SEQ |
|---|---|---|---|
| | | | ID |
| Name | Description | Amino Acid Sequence | NO: |
| BMA031 | TCR CAR | MALPVTALLLPLALLLHAARPQIVLTQSPAIMSASPGEKVTMTCSATS | 110 |
| CAR | | SVSYMHWYQQKSGTSPKRWIYDTSKLASGVPARFSGSGSGTSYSLTIS | |
| | | SMEAEDAATYYCQQWSSNPLTFGAGTKLELKGGGGSGGGGSGGGGS | |
| | | GGGGSEVQLQQSGPELVKPGASVKMSCKASGYKFTSYVMHWVKQK | |
| | | PGQGLEWIGYINPYNDVTKYNEKFKGKATLTSDKSSSTAYMELSSLTS | |
| | | EDSAVHYCARGSYYDYDGFVYWGQGTLVTVSAKPTTTPAPRPPTPAP | |
| | | TIASQPLSLRPEACRPAAGGAVHTRGLDFACDIYIWAPLAGTCGVLLL | |
| | | SLVITLYCKRGRKKLLYIFKQPFMRPVQTTQEEDGCSCRFPEEEEGGCE | |
| | | LRVKFSRSADAPAYQQGQNQLYNELNLGRREEYDVLDKRRGRDPEM | |
| | | GGKPRRKNPQEGLYNELQKDKMAEAYSEIGMKGERRRGKGHDGLY | |
| | | QGLSTATKDTYDALHMQALPPR | |
| | TCR CAR | QIVLTQSPAIMSASPGEKVTMTCSATSSVSYMHWYQQKSGTSPKRWI | 111 |
| | without | YDTSKLASGVPARFSGSGSGTSYSLTISSMEAEDAATYYCQQWSSNPL | |
| | signal | TFGAGTKLELKGGGGSGGGGSGGGGSGGGGSEVQLQQSGPELVKPG | |
| | peptide | ASVKMSCKASGYKFTSYVMHWVKQKPGQGLEWIGYINPYNDVTKY | |
| | | NEKFKGKATLTSDKSSSTAYMELSSLTSEDSAVHYCARGSYYDYDGF | |
| | | VYWGQGTLVTVSAKPTTTPAPRPPTPAPTIASQPLSLRPEACRPAAGG | |
| | | AVHTRGLDFACDIYIWAPLAGTCGVLLLSLVITLYCKRGRKKLLYIFK | |
| | | QPFMRPVQTTQEEDGCSCRFPEEEEGGCELRVKFSRSADAPAYQQGQ | |
| | | NQLYNELNLGRREEYDVLDKRRGRDPEMGGKPRRKNPQEGLYNELQ | |
| | | KDKMAEAYSEIGMKGERRRGKGHDGLYQGLSTATKDTYDALHMQA | |
| | | LPPR | |
| | CD8α | MALPVTALLLPLALLLHAARP | 97 |
| | signal | | |
| | peptide | | |
| | scFv | QIVLTQSPAIMSASPGEKVTMTCSATSSVSYMHWYQQKSGTSPKRWI | 112 |
| | | YDTSKLASGVPARFSGSGSGTSYSLTISSMEAEDAATYYCQQWSSNPL | |
| | | TFGAGTKLELKGGGGSGGGGSGGGGSGGGGSEVQLQQSGPELVKPG | |
| | | ASVKMSCKASGYKFTSYVMHWVKQKPGQGLEWIGYINPYNDVTKY | |
| | | NEKFKGKATLTSDKSSSTAYMELSSLTSEDSAVHYCARGSYYDYDGF | |
| | | VYWGQGTLVTVSA | |
| | VH | EVQLQQSGPELVKPGASVKMSCKASGYKFTSYVMHWVKQKPGQGLE | 7 |
| | | WIGYINPYNDVTKYNEKFKGKATLTSDKSSSTAYMELSSLTSEDSAVH | |
| | | YCARGSYYDYDGFVYWGQGTLVTVSA | |
| | VL | QIVLTQSPAIMSASPGEKVTMTCSATSSVSYMHWYQQKSGTSPKRWI | 8 |
| | | YDTSKLASGVPARFSGSGSGTSYSLTISSMEAEDAATYYCQQWSSNPL | |
| | | TFGAGTKLELK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACDIYI | 101 |
| | hinge and | WAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPEMG | 103 |
| | signaling | GKPRRKNPQEGLYNELQKDKMAEAYSEIGMKGERRRGKGHDGLYQ | |
| | domain of | GLSTATKDTYDALHMQALPPR | |
| | CD3ζ | | |
| CIV3 | CIV3 CAR | MALPVTALLLPLALLLHAARPDIQMTQSPSTLSASVGDRVTMTCSATS | 113 |
| CAR | | SVSYMHWYQQKPGKAPKRWIYDTSKLASGVPARFIGSGSGTEFTLTIS | |
| | | SLQPDDFATYYCQQWSSNPLTFGGGTKVEIKGGGGSGGGGSGGGGSG | |
| | | GGGSQVQLVQSGAEVKKPGSSVKVSCKASGYKFTSYVMHWVKQAP | |
| | | GQGLEWIGYINPYNDVTKYNEKFKGKATLTADESTNTAYMELSSLRS | |
| | | EDTAVHYCARGSYYDYDGFVYWGQGTLVTVSSKPTTTPAPRPPTPAP | |
| | | TIASQPLSLRPEACRPAAGGAVHTRGLDFACDIYIWAPLAGTCGVLLL | |
| | | SLVITLYCKRGRKKLLYIFKQPFMRPVQTTQEEDGCSCRFPEEEEGGCE | |
| | | LRVKFSRSADAPAYQQGQNQLYNELNLGRREEYDVLDKRRGRDPEM | |
| | | GGKPRRKNPQEGLYNELQKDKMAEAYSEIGMKGERRRGKGHDGLY | |
| | | QGLSTATKDTYDALHMQALPPR | |
| | CIV3 CAR | DIQMTQSPSTLSASVGDRVTMTCSATSSVSYMHWYQQKPGKAPKRWI | 114 |
| | without | YDTSKLASGVPARFIGSGSGTEFTLTISSLQPDDFATYYCQQWSSNPLT | |
| | signal | FGGGTKVEIKGGGGSGGGGSGGGGSGGGGSQVQLVQSGAEVKKPGS | |
| | peptide | SVKVSCKASGYKFTSYVMHWVKQAPGQGLEWIGYINPYNDVTKYNE | |
| | | KFKGKATLTADESTNTAYMELSSLRSEDTAVHYCARGSYYDYDGFV | |
| | | YWGQGTLVTVSSKPTTTPAPRPPTPAPTIASQPLSLRPEACRPAAGGAV | |
| | | HTRGLDFACDIYIWAPLAGTCGVLLLSLVITLYCKRGRKKLLYIFKQPF | |
| | | MRPVQTTQEEDGCSCRFPEEEEGGCELRVKFSRSADAPAYQQGQNQL | |
| | | YNELNLGRREEYDVLDKRRGRDPEMGGKPRRKNPQEGLYNELQKDK | |
| | | MAEAYSEIGMKGERRRGKGHDGLYQGLSTATKDTYDALHMQALPPR | |
| | CD8α | MALPVTALLLPLALLLHAARP | 97 |
| | signal | | |
| | peptide | | |
| | scFv | DIQMTQSPSTLSASVGDRVTMTCSATSSVSYMHWYQQKPGKAPKRWI | 115 |
| | | YDTSKLASGVPARFIGSGSGTEFTLTISSLQPDDFATYYCQQWSSNPLT | |
| | | FGGGTKVEIKGGGGSGGGGSGGGGSGGGGSQVQLVQSGAEVKKPGS | |
| | | SVKVSCKASGYKFTSYVMHWVKQAPGQGLEWIGYINPYNDVTKYNE | |
| | | KFKGKATLTADESTNTAYMELSSLRSEDTAVHYCARGSYYDYDGFV | |
| | | YWGQGTLVTVSS | |
| | VH | QVQLVQSGAEVKKPGSSVKVSCKASGYKFTSYVMHWVKQAPGQGL | 9 |
| | | EWIGYINPYNDVTKYNEKFKGKATLTADESTNTAYMELSSLRSEDTA | |
| | | VHYCARGSYYDYDGFVYWGQGTLVTVSS | |
| | VL | DIQMTQSPSTLSASVGDRVTMTCSATSSVSYMHWYQQKPGKAPKRWI | 10 |
| | | YDTSKLASGVPARFIGSGSGTEFTLTISSLQPDDFATYYCQQWSSNPLT | |
| | | FGGGTKVEIK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACDIYI | 101 |
| | hinge and | WAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPEMG | 103 |
| | signaling | GKPRRKNPQEGLYNELQKDKMAEAYSEIGMKGERRRGKGHDGLYQ | |
| | domain of | GLSTATKDTYDALHMQALPPR | |
| | CD3ζ | | |
| TOL101 | TOL101 | MALPVTALLLPLALLLHAARPQIVLTQSPAIMSASPGEKVTMTCSASSS | 116 |
| CAR | CAR | VSYMHWYQQKSGTSPKRWIYDTSKLASGVPARFSGSGSGTSYSLTISS | |
| | | MEAEDAATYYCQQWSSNPFTFGSGTKLEIKRAGGGGSGGGGSGGGG | |
| | | SGGGGSQVQLQQSGAELARPGASVKMSCKASGYTFTSYTMHWVKQR | |
| | | PGQGLEWIGYINPSSGYTNYNQKFKDKATLTADKSSSTAYMQLSSLTS | |
| | | EDSAVYYCARWRDAYYAMDYWGQGTSVTVSSKPTTTPAPRPPTPAP | |
| | | TIASQPLSLRPEACRPAAGGAVHTRGLDFACDIYIWAPLAGTCGVLLL | |
| | | SLVITLYCKRGRKKLLYIFKQPFMRPVQTTQEEDGCSCRFPEEEEGGCE | |
| | | LRVKFSRSADAPAYQQGQNQLYNELNLGRREEYDVLDKRRGRDPEM | |
| | | GGKPRRKNPQEGLYNELQKDKMAEAYSEIGMKGERRRGKGHDGLY | |
| | | QGLSTATKDTYDALHMQALPPR | |
| | TOL101 | QIVLTQSPAIMSASPGEKVTMTCSASSSVSYMHWYQQKSGTSPKRWI | 117 |
| | CAR | YDTSKLASGVPARFSGSGSGTSYSLTISSMEAEDAATYYCQQWSSNPF | |
| | without | TFGSGTKLEIKRAGGGGSGGGGSGGGGSGGGGSQVQLQQSGAELARP | |
| | signal | GASVKMSCKASGYTFTSYTMHWVKQRPGQGLEWIGYINPSSGYTNY | |
| | peptide | NQKFKDKATLTADKSSSTAYMQLSSLTSEDSAVYYCARWRDAYYAM | |
| | | DYWGQGTSVTVSSKPTTTPAPRPPTPAPTIASQPLSLRPEACRPAAGGA | |
| | | VHTRGLDFACDIYIWAPLAGTCGVLLLSLVITLYCKRGRKKLLYIFKQ | |
| | | PFMRPVQTTQEEDGCSCRFPEEEEGGCELRVKFSRSADAPAYQQGQN | |
| | | QLYNELNLGRREEYDVLDKRRGRDPEMGGKPRRKNPQEGLYNELQK | |
| | | DKMAEAYSEIGMKGERRRGKGHDGLYQGLSTATKDTYDALHMQAL | |
| | | PPR | |
| | CD8α | MALPVTALLLPLALLLHAARP | 97 |
| | signal | | |
| | peptide | | |
| | scFv | QIVLTQSPAIMSASPGEKVTMTCSASSSVSYMHWYQQKSGTSPKRWI | 118 |
| | | YDTSKLASGVPARFSGSGSGTSYSLTISSMEAEDAATYYCQQWSSNPF | |
| | | TFGSGTKLEIKRAGGGGSGGGGSGGGGSGGGGSQVQLQQSGAELARP | |
| | | GASVKMSCKASGYTFTSYTMHWVKQRPGQGLEWIGYINPSSGYTNY | |
| | | NQKFKDKATLTADKSSSTAYMQLSSLTSEDSAVYYCARWRDAYYAM | |
| | | DYWGQGTSVTVSS | |
| | VH | QVQLQQSGAELARPGASVKMSCKASGYTFTSYTMHWVKQRPGQGLE | 11 |
| | | WIGYINPSSGYTNYNQKFKDKATLTADKSSSTAYMQLSSLTSEDSAVY | |
| | | YCARWRDAYYAMDYWGQGTSVTVSS | |
| | VL | QIVLTQSPAIMSASPGEKVTMTCSASSSVSYMHWYQQKSGTSPKRWI | 12 |
| | | YDTSKLASGVPARFSGSGSGTSYSLTISSMEAEDAATYYCQQWSSNPF | |
| | | TFGSGTKLEIK | |
| | scFv | RAGGGGSGGGGSGGGGSGGGGS | 119 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACDIYI | 101 |
| | hinge and | WAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPEMG | 103 |
| | signaling | GKPRRKNPQEGLYNELQKDKMAEAYSEIGMKGERRRGKGHDGLYQ | |
| | domain of | GLSTATKDTYDALHMQALPPR | |
| | CD3ζ | | |

In some embodiments, the engineered cell may be modified to prevent expression of cell surface antigens. In some embodiments, the engineered cell includes an inducible kill gene (“safety switch”) or a combination of safety switches, which may be assembled on a vector, such as, without limiting, a retroviral vector, lentiviral vector, adenoviral vector or plasmid. The safety switch can include, for example, an apoptosis inducing signaling cascade or a drug that induces cell death. In some embodiments, the elimination of unwanted modified T cells involve expression of CD20, e.g., truncated CD20, CD19, or truncated epidermal growth factor receptor in T cells. For example, in the presence of anti-CD20 antibody such as rituximab, cells expressing the CD20 kill gene can be efficiently eliminated by complement dependent cytotoxicity induced by the anti-CD20 antibody such as rituximab. In the presence of anti-CD20 antibody such as rituximab, cells expressing the CD20 kill gene can be efficiently eliminated by antibody-dependent cellular cytotoxicity induced by the anti-CD20 antibody such as rituximab In some embodiments, the “safety switch” may be an inducible kill gene, such as, without limiting, caspase 9, herpes simplex virus thymidine kinase, cytosine deaminase (CD) or cytochrome P450. Other known safety switches have been contemplated and are embodied in the present invention.

In some embodiments, a kill gene is integrated into the engineered cell genome. In some embodiments, a CAR-expressing cell described herein may also express a target protein recognized by a T cell depleting agent. In one embodiment, the target protein is CD20 and the T cell depleting agent is an anti-CD20 antibody, e.g., rituximab. In such embodiment, the T cell depleting agent is administered when it is desirable to reduce or eliminate the CAR-expressing cell, e.g., to mitigate the CAR induced toxicity.

In some embodiments, the present disclosure provides an engineered cell having a nucleotide having a first polynucleotide encoding a CAR (CD3 CAR or TCR CAR) linked to second polynucleotide encoding a CD20 protein, e.g., a truncated CD20 protein having an amino acid sequence of SEQ ID NO: 120 or SEQ ID NO: 121. In some embodiments, the nucleic acid sequences encoding the CAR and the CD20 are situated in the same orientation, e.g., transcription of the nucleic acid sequences encoding the CAR and the CD20 proceeds in the same direction. In some embodiments, the nucleic acid sequences encoding the CAR and the CD20 are situated in different orientations. In some embodiments, a single promoter controls expression of the nucleic acid sequences encoding the CAR and the CD20.

In some embodiments, an engineered cell may comprise a CAR (e.g., CD3 CAR), a PEBL (e.g., CD3 PEBL), and a kill gene (e.g., CD20t). A cell comprising the CD3 CAR, CD3 PEBL, and CD20t kill gene may be referred to as a PCART^{KG} cell. The terms “PCART^{KG}” and “CD3 PEBL CD3 CAR-CD20t T cells” may be used interchangeably.

In some embodiments, a nucleic acid encoding a “self-cleaving” ribosomal skipping site (such as a T2A, P2A, E2A, or F2A) is situated between the nucleic acid sequences encoding the CAR (CD3 CAR or TCR CAR) and the CD20. See. e.g., Liu et al, Scientific Rports 7: 2193, 2017. In some embodiments, the nucleic acid sequences encoding the CD3 CAR is upstream of the nucleic acid sequences encoding the CD20, or the nucleic acid sequences encoding the CD20 is upstream of the nucleic acid sequences encoding the CAR. In some embodiments, a first promoter controls expression of the nucleic acid sequence encoding the CAR and a second promoter controls expression of the nucleic acid sequence encoding the CD20. In some embodiments, the two open reading frames are linked by an internal ribosome entry site (IRES). In some embodiments, the nucleic acid comprises a viral packaging element. In some aspects, the present disclosure provides a cell, e.g., an immune cell, comprising the nucleic acid described herein, e.g., a nucleic acid comprising the CAR and the CD20 protein as described above. In some aspects, the present disclosure provides, e.g., a composition comprising: (i) a first polynucleotide encoding a CAR molecule, e.g., a CAR molecule that binds CD3 or TCR described herein and (ii) a second polynucleotide encoding a CD20 protein as described herein.

In some embodiments, the CD20 protein has an amino acid sequence as set forth in SEQ ID NO: 123 or SEQ ID NO: 124. In some embodiments, the CAR is linked to the CD20 protein via a self-cleaving peptide, e.g., a P2A self-cleaving peptide, e.g., having an amino acid sequence as set forth in SEQ ID NO: 122. In some embodiments, the C-terminus of the first polynucleotide is linked to the N-terminus of the second polynucleotide via P2A linker. In some embodiments, the N-terminus of the first polynucleotide is linked to the C-terminus of the second polynucleotide via P2A self-cleaving peptide.

The full-length CD20 protein sequence can be

(SEQ ID NO: 123)
MTTPRNSVNGTFPAEPMKGPIAMQSGPKPLFRRMSSLVGPTQSFFMRESK
TLGAVQIMNGLFHIALGGLLMIPAGIYAPICVTVWYPLWGGIMYIISGSL
LAATEKNSRKCLVKGKMIMNSLSLFAAISGMILSIMDILNIKISHFLKME
SLNFIRAHTPYINIYNCEPANPSEKNSPSTQYCYSIQSLFLGILSVMLIF
AFFQELVIAGIVENEWKRTCSRPKSNIVLLSAEEKKEQTIEIKEEVVGLT
ETSSQPKNEEDIEIIPIQEEEEEETETNFPEPPQDQESSPIENDSSP

In some embodiments, the CD20 protein is truncated, e.g., truncated after E263. In some embodiments, the truncated CD20 protein lacks amino acid 264-297 compared to a full-length CD20 protein.

TABLE 9
Amino acid sequences of exemplary CD3 CAR polypeptides with a kill gene and
select components

| | | | SEQ |
|---|---|---|---|
| | | | ID |
| | Description | Amino Acid Sequence | NO: |
| Anti- | CD3 CAR | MALPVTALLLPLALLLHAARPDIQMTQTTSSLSASLGDRVTISCRAS | 95 |
| CD3 | | QDIRNYLNWYQQKPDGTVKLLIYYTSRLHSGVPSKFSGSGSGTDYS | |
| (UCHT1) | | LTISNLEQEDIATYFCQQGNTLPWTFAGGTKLEIKGGGGSGGGGSG | |
| CAR | | GGGSGGGGSEVQLQQSGPELVKPGASMKISCKASGYSFTGYTMN | |
| | | WVKQSHGKNLEWMGLINPYKGVSTYNQKFKDKATLTVDKSSSTA | |
| | | YMELLSLTSEDSAVYYCARSGYYGDSDWYFDVWGQGTTLTVFSK | |
| | | PTTTPAPRPPTPAPTIASQPLSLRPEACRPAAGGAVHTRGLDFACDI | |
| | | YIWAPLAGTCGVLLLSLVITLYCKRGRKKLLYIFKQPFMRPVQTTQ | |
| | | EEDGCSCRFPEEEEGGCELRVKFSRSADAPAYQQGQNQLYNELNL | |
| | | GRREEYDVLDKRRGRDPEMGGKPRRKNPQEGLYNELQKDKMAE | |
| | | AYSEIGMKGERRRGKGHDGLYQGLSTATKDTYDALHMQALPPR | |
| | CD3 CAR | DIQMTQTTSSLSASLGDRVTISCRASQDIRNYLNWYQQKPDGTVKL | 96 |
| | without | LIYYTSRLHSGVPSKFSGSGSGTDYSLTISNLEQEDIATYFCQQGNT | |
| | signal | LPWTFAGGTKLEIKGGGGSGGGGSGGGGSGGGGSEVQLQQSGPEL | |
| | peptide | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFSKPTTTPAPRPPTPAPTIASQPLSL | |
| | | RPEACRPAAGGAVHTRGLDFACDIYIWAPLAGTCGVLLLSLVITLY | |
| | | CKRGRKKLLYIFKQPFMRPVQTTQEEDGCSCRFPEEEEGGCELRVK | |
| | | FSRSADAPAYQQGQNQLYNELNLGRREEYDVLDKRRGRDPEMGG | |
| | | KPRRKNPQEGLYNELQKDKMAEAYSEIGMKGERRRGKGHDGLYQ | |
| | | GLSTATKDTYDALHMQALPPR | |
| | CD8α | MALPVTALLLPLALLLHAARP | 97 |
| | signal | | |
| | peptide | | |
| | scFv | DIQMTQTTSSLSASLGDRVTISCRASQDIRNYLNWYQQKPDGTVKL | 98 |
| | | LIYYTSRLHSGVPSKFSGSGSGTDYSLTISNLEQEDIATYFCQQGNT | |
| | | LPWTFAGGTKLEIKGGGGSGGGGSGGGGSGGGGSEVQLQQSGPEL | |
| | | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFS | |
| | VH | EVQLQQSGPELVKPGASMKISCKASGYSFTGYTMNWVKQSHGKN | 1 |
| | | LEWMGLINPYKGVSTYNQKFKDKATLTVDKSSSTAYMELLSLTSE | |
| | | DSAVYYCARSGYYGDSDWYFDVWGQGTTLTVFS | |
| | VL | DIQMTQTTSSLSASLGDRVTISCRASQDIRNYLNWYQQKPDGTVKL | 2 |
| | | LIYYTSRLHSGVPSKFSGSGSGTDYSLTISNLEQEDIATYFCQQGNT | |
| | | LPWTFAGGTKLEIK | |
| | scFv | GGGGGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACD | 101 |
| | hinge and | IYIWAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPE | 103 |
| | signaling | MGGKPRRKNPQEGLYNELQKDKMAEAYSEIGMKGERRRGKGHD | |
| | domain of | GLYQGLSTATKDTYDALHMQALPPR | |
| | CD3ζ | | |
| Anti- | CD3 CAR | MALPVTALLLPLALLLHAARPQIVLTQSPAIMSASPGEKVTMTCSA | 104 |
| CD3 | | SSSVSYMNWYQQKSGTSPKRWIYDTSKLASGVPAHFRGSGSGTSY | |
| (OKT3) | | SLTISGMEAEDAATYYCQQWSSNPFTFGSGTKLEINRGGGGSGGG | |
| CAR | | GSGGGGSGGGGSEVQLQQSGAELARPGASVKMSCKASGYTFTRY | |
| | | TMHWVKQRPGQGLEWIGYINPSRGYTNYNQKFKDKATLTTDKSSS | |
| | | TAYMQLSSLTSEDSAVYYCARYYDDHYCLDYWGQGTTLTVSSAK | |
| | | PTTTPAPRPPTPAPTIASQPLSLRPEACRPAAGGAVHTRGLDFACDI | |
| | | YIWAPLAGTCGVLLLSLVITLYCKRGRKKLLYIFKQPFMRPVQTTQ | |
| | | EEDGCSCRFPEEEEGGCELRVKFSRSADAPAYQQGQNQLYNELNL | |
| | | GRREEYDVLDKRRGRDPEMGGKPRRKNPQEGLYNELQKDKMAE | |
| | | AYSEIGMKGERRRGKGHDGLYQGLSTATKDTYDALHMQALPPR | |
| | CD3 CAR | QIVLTQSPAIMSASPGEKVTMTCSASSSVSYMNWYQQKSGTSPKR | 105 |
| | without | WIYDTSKLASGVPAHFRGSGSGTSYSLTISGMEAEDAATYYCQQW | |
| | signal | SSNPFTFGSGTKLEINRGGGGSGGGGSGGGGSGGGGSEVQLQQSG | |
| | peptide | AELARPGASVKMSCKASGYTFTRYTMHWVKQRPGQGLEWIGYIN | |
| | | PSRGYTNYNQKFKDKATLTTDKSSSTAYMQLSSLTSEDSAVYYCA | |
| | | RYYDDHYCLDYWGQGTTLTVSSAKPTTTPAPRPPTPAPTIASQPLS | |
| | | LRPEACRPAAGGAVHTRGLDFACDIYIWAPLAGTCGVLLLSLVITL | |
| | | YCKRGRKKLLYIFKQPFMRPVQTTQEEDGCCRFPEEEEGGCELRV | |
| | | KFSRSADAPAYQQGQNQLYNELNLGRREEYDVLDKRRGRDPEMG | |
| | | GKPRRKNPQEGLYNELQKDKMAEAYSEIGMKGERRRGKGHDGLY | |
| | | QGLSTATKDTYDALHMQALPPR | |
| | CD8α | MALPVTALLLPLALLLHAARP | 97 |
| | signal | | |
| | peptide | | |
| | scFv | QIVLTQSPAIMSASPGEKVTMTCSASSSVSYMNWYQQKSGTSPKR | 106 |
| | | WIYDTSKLASGVPAHFRGSGSGTSYSLTISGMEAEDAATYYCQQW | |
| | | SSNPFTFGSGTKLEINRGGGGSGGGGSGGGGSGGGGSEVQLQQSG | |
| | | AELARPGASVKMSCKASGYTFTRYTMHWVKQRPGQGLEWIGYIN | |
| | | PSRGYTNYNQKFKDKATLTTDKSSSTAYMQLSSLTSEDSAVYYCA | |
| | | RYYDDHYCLDYWGQGTTLTVSSA | |
| | VH | EVQLQQSGAELARPGASVKMSCKASGYTFTRYTMHWVKQRPGQG | 3 |
| | | LEWIGYINPSRGYTNYNQKFKDKATLTTDKSSSTAYMQLSSLTSED | |
| | | SAVYYCARYYDDHYCLDYWGQGTTLTVSSA | |
| | VL | QIVLTQSPAIMSASPGEKVTMTCSASSSVSYMNWYQQKSGTSPKR | 4 |
| | | WIYDTSKLASGVPAHFRGSGSGTSYSLTISGMEAEDAATYYCQQW | |
| | | SSNPFTFGSGTKLEINR | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACD | 101 |
| | hinge and | IYIWAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPE | 103 |
| | signaling | MGGKPRRKNPQEGLYNELQKDKMAEAYSEIGMKGERRRGKGHD | |
| | domain of | GLYQGLSTATKDTYDALHMQALPPR | |
| | CD3ζ | | |
| Anti- | CD3 CAR | MALPVTALLLPLALLLHAARPEIVLTQSPATLSLSPGERATLSCRAS | 107 |
| CD3 | | QSVSSYLAWYQQKPGQAPRLLIYDASNRATGIPARFSGSGSGTDFT | |
| (28F11) | | LTISSLEPEDFAVYYCQQRSNWPPLTFGGGTKVEIKGGGGSGGGGS | |
| CAR | | GGGGSGGGGSQVQLVESGGGVVQPGRSLRLSCAASGFKFSGYGM | |
| | | HWVRQAPGKGLEWVAVIWYDGSKKYYVDSVKGRFTISRDNSKNT | |
| | | LYLQMNSLRAEDTAVYYCARQMGYWHFDLWGRGTLVTVSSKPT | |
| | | TTPAPRPPTPAPTIASQPLSLRPEACRPAAGGAVHTRGLDFACDIYI | |
| | | WAPLAGTCGVLLLSLVITLYCKRGRKKLLYIFKQPFMRPVQTTQEE | |
| | | DGCSCRFPEEEEGGCELRVKFSRSADAPAYQQGQNQLYNELNLGR | |
| | | REEYDVLDKRRGRDPEMGGKPRRKNPQEGLYNELQKDKMAEAYS | |
| | | EIGMKGERRRGKGHDGLYQGLSTATKDTYDALHMQALPPR | |
| | CD3 CAR | EIVLTQSPATLSLSPGERATLSCRASQSVSSYLAWYQQKPGQAPRL | 108 |
| | without | LIYDASNRATGIPARFSGSGSGTDFTLTISSLEPEDFAVYYCQQRSN | |
| | signal | WPPLTFGGGTKVEIKGGGGSGGGGSGGGGSGGGGSQVQLVESGG | |
| | peptide | GVVQPGRSLRLSCAASGFKFSGYGMHWVRQAPGKGLEWVAVIW | |
| | | YDGSKKYYVDSVKGRFTISRDNSKNTLYLQMNSLRAEDTAVYYC | |
| | | ARQMGYWHFDLWGRGTLVTVSSKPTTTPAPRPPTPAPTIASQPLSL | |
| | | RPEACRPAAGGAVHTRGLDFACDIYIWAPLAGTCGVLLLSLVITLY | |
| | | CKRGRKKLLYIFKQPFMRPVQTTQEEDGCSCRFPEEEEGGCELRVK | |
| | | FSRSADAPAYQQGQNQLYNELNLGRREEYDVLDKRRGRDPEMGG | |
| | | KPRRKNPQEGLYNELQKDKMAEAYSEIGMKGERRRGKGHDGLYQ | |
| | | GLSTATKDTYDALHMQALPPR | |
| | CD8α | MALPVTALLLPLALLLHAARP | 97 |
| | signal | | |
| | peptide | | |
| | scFv | EIVLTQSPATLSLSPGERATLSCRASQSVSSYLAWYQQKPGQAPRL | 109 |
| | | LIYDASNRATGIPARFSGSGSGTDFTLTISSLEPEDFAVYYCQQRSN | |
| | | WPPLTFGGGTKVEIKGGGGSGGGGSGGGGSGGGGSQVQLVESGG | |
| | | GVVQPGRSLRLSCAASGFKFSGYGMHWVRQAPGKGLEWVAVIW | |
| | | YDGSKKYYVDSVKGRFTISRDNSKNTLYLQMNSLRAEDTAVYYC | |
| | | ARQMGYWHFDLWGRGTLVTVSS | |
| | VH | QVQLVESGGGVVQPGRSLRLSCAASGFKFSGYGMHWVRQAPGKG | 5 |
| | | LEWVAVIWYDGSKKYYVDSVKGRFTISRDNSKNTLYLQMNSLRA | |
| | | EDTAVYYCARQMGYWHFDLWGRGTLVTVSS | |
| | VL | EIVLTQSPATLSLSPGERATLSCRASQSVSSYLAWYQQKPGQAPRL | 6 |
| | | LIYDASNRATGIPARFSGSGSGTDFTLTISSLEPEDFAVYYCQQRSN | |
| | | WPPLTFGGGTKVEIK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACD | 101 |
| | hinge and | IYIWAPLAGTCGVLLLSLVITLYC | |
| | transmembrane | | |
| | domain | | |
| | Costimulatory | KRGRKKLLYIFKQPFMRPVQTTQEEDGCCRFPEEEEGGCEL | 102 |
| | domain of | | |
| | 4-1BB | | |
| | Primary | RVKFSRSADAPAYQQGQNQLYNELNLGRREEYDVLDKRRGRDPE | 103 |
| | signaling | MGGKPRRKNPQEGLYNELQKDKMAEAYSEIGMKGERRRGKGHD | |
| | domain of | GLYQGLSTATKDTYDALHMQALPPR | |
| | CD3ζ | | |
| Anti- | CD3 CAR- | MALPVTALLLPLALLLHAARPDIQMTQTTSSLSASLGDRVTISCRAS | 120 |
| CD3 | P2A- | QDIRNYLNWYQQKPDGTVKLLIYYTSRLHSGVPSKFSGSGSGTDYS | |
| (CD8sp- | CD20t | LTISNLEQEDIATYFCQQGNTLPWTFAGGTKLEIKGGGGSGGGGSG | |
| UCHT1- | | GGGSGGGGSEVQLQQSGPELVKPGASMKISCKASGYSFTGYTMN | |
| CD8 | | WVKQSHGKNLEWMGLINPYKGVSTYNQKFKDKATLTVDKSSSTA | |
| hinge | | YMELLSLTSEDSAVYYCARSGYYGDSDWYFDVWGQGTTLTVFSK | |
| and | | PTTTPAPRPPTPAPTIASQPLSLRPEACRPAAGGAVHTRGLDFACDI | |
| trans- | | YIWAPLAGTCGVLLLSLVITLYCKRGRKKLLYIFKQPFMRPVQTTQ | |
| membrane- | | EEDGCSCRFPEEEEGGCELRVKFSRSADAPAYQQGQNQLYNELNL | |
| 41BB- | | GRREEYDVLDKRRGRDPEMGGKPRRKNPQEGLYNELQKDKMAE | |
| CD3zeta- | | AYSEIGMKGERRRGKGHDGLYQGLSTATKDTYDALHMQALPPRG | |
| GSGP2A- | | SGATNFSLLKQAGDVEENPGPMTTPRNSVNGTFPAEPMKGPIAMQ | |
| CD20t) | | SGPKPLFRRMSSLVGPTQSFFMRESKTLGAVQIMNGLFHIALGGLL | |
| CAR | | MIPAGIYAPICVTVWYPLWGGIMYIISGSLLAATEKNSRKCLVKGK | |
| | | MIMNSLSLFAAISGMILSIMDILNIKISHFLKMESLNFIRAHTPYINIY | |
| | | NCEPANPSEKNSPSTQYCYSIQSLFLGILSVMLIFAFFQELVIAGIVE | |
| | | NEWKRTCSRPKSNIVLLSAEEKKEQTIEIKEEVVGLTETSSQPKNEE | |
| | | DIE | |
| | CD3 CAR- | DIQMTQTTSSLSASLGDRVTISCRASQDIRNYLNWYQQKPDGTVKL | 121 |
| | P2A- | LIYYTSRLHSGVPSKFSGSGSGTDYSLTISNLEQEDIATYFCQQGNT | |
| | CD20t | LPWTFAGGTKLEIKGGGGSGGGGSGGGGSGGGGSEVQLQQSGPEL | |
| | without | VKPGASMKISCKASGYSFTGYTMNWVKQSHGKNLEWMGLINPYK | |
| | signal | GVSTYNQKFKDKATLTVDKSSSTAYMELLSLTSEDSAVYYCARSG | |
| | peptide | YYGDSDWYFDVWGQGTTLTVFSKPTTTPAPRPPTPAPTIASQPLSL | |
| | | RPEACRPAAGGAVHTRGLDFACDIYIWAPLAGTCGVLLLSLVITLY | |
| | | CKRGRKKLLYIFKQPFMRPVQTTQEEDGCSCRFPEEEEGGCELRVK | |
| | | FSRSADAPAYQQGQNQLYNELNLGRREEYDVLDKRRGRDPEMGG | |
| | | KPRRKNPQEGLYNELQKDKMAEAYSEIGMKGERRRGKGHDGLYQ | |
| | | GLSTATKDTYDALHMQALPPRGSGATNFSLLKQAGDVEENPGPMT | |
| | | TPRNSVNGTFPAEPMKGPIAMQSGPKPLFRRMSSLVGPTQSFFMRE | |
| | | SKTLGAVQIMNGLFHIALGGLLMIPAGIYAPICVTVWYPLWGGIMY | |
| | | IISGSLLAATEKNSRKCLVKGKMIMNSLSLFAAISGMILSIMDILNIKI | |
| | | SHFLKMESLNFIRAHTPYINIYNCEPANPSEKNSPSTQYCYSIQSLFL | |
| | | GILSVMLIFAFFQELVIAGIVENEWKRTCSRPKSNIVLLSAEEKKEQT | |
| | | IEIKEEVVGLTETSSQPKNEEDIE | |
| | CD8α | MALPVTALLLPLALLLHAARP | 97 |
| | signal | | |
| | peptide | | |
| | scFv | DIQMTQTTSSLSASLGDRVTISCRASQDIRNYLNWYQQKPDGTVKL | 98 |
| | | LIYYTSRLHSGVPSKFSGSGSGTDYSLTISNLEQEDIATYFCQQGNT | |
| | | LPWTFAGGTKLEIKGGGGSGGGGSGGGGSGGGGSEVQLQQSGPEL | |
| | | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFS | |
| | VH | EVQLQQSGPELVKPGASMKISCKASGYSFTGYTMNWVKQSHGKN | 1 |
| | | LEWMGLINPYKGVSTYNQKFKDKATLTVDKSSSTAYMELLSLTSE | |
| | | DSAVYYCARSGYYGDSDWYFDVWGQGTTLTVFS | |
| | VL | DIQMTQTTSSLSASLGDRVTISCRASQDIRNYLNWYQQKPDGTVKL | 2 |
| | | LIYYTSRLHSGVPSKFSGSGSGTDYSLTISNLEQEDIATYFCQQGNT | |
| | | LPWTFAGGTKLEIK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACD | 101 |
| | hinge and | IYIWAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPE | 103 |
| | signaling | MGGKPRRKNPQEGLYNELQKDKMAEAYSEIGMKGERRRGKGHD | |
| | domain of | GLYQGLSTATKDTYDALHMQALPPR | |
| | CD3ζ | | |
| | GSG-P2A | GSGATNFSLLKQAGDVEENPGP | 122 |
| | CD20t* | MTTPRNSVNGTFPAEPMKGPIAMQSGPKPLFRRMSSLVGPTQSFFM | 124 |
| | | RESKTLGAVQIMNGLFHIALGGLLMIPAGIYAPICVTVWYPLWGGI | |
| | | MYIISGSLLAATEKNSRKCLVKGKMIMNSLSLFAAISGMILSIMDIL | |
| | | NIKISHFLKMESLNFIRAHTPYINIYNCEPANPSEKNSPSTQYCYSIQS | |
| | | LFLGILSVMLIFAFFQELVIAGIVENEWKRTCSRPKSNIVLLSAEEKK | |
| | | EQTIEIKEEVVGLTETSSQPKNEEDIE | |
| Anti- | CD3 CAR | MALPVTALLLPLALLLHAARPDIQMTQTTSSLSASLGDRVTISCRAS | 125 |
| CD3 | | QDIRNYLNWYQQKPDGTVKLLIYYTSRLHSGVPSKFSGSGSGTDYS | |
| (UCHT1- | | LTISNLEQEDIATYFCQQGNTLPWTFAGGTKLEIKGGGGSGGGGSG | |
| CD8 | | GGGSGGGGSEVQLQQSGPELVKPGASMKISCKASGYSFTGYTMN | |
| hinge- | | WVKQSHGKNLEWMGLINPYKGVSTYNQKFKDKATLTVDKSSSTA | |
| 41BB- | | YMELLSLTSEDSAVYYCARSGYYGDSDWYFDVWGQGTTLTVFSK | |
| CD3zeta- | | PTTTPAPRPPTPAPTIASQPLSLRPEACRPAAGGAVHTRGLDFACDI | |
| IRES- | | YIWAPLAGTCGVLLLSLVITLYCKRGRKKLLYIFKQPFMRPVQTTQ | |
| CD20t) | | EEDGCSCRFPEEEEGGCELRVKFSRSADAPAYQQGQNQLYNELNL | |
| CAR | | GRREEYDVLDKRRGRDPEMGGKPRRKNPQEGLYNELQKDKMAE | |
| | | AYSEIGMKGERRRGKGHDGLYQGLSTATKDTYDALHMQALPPR | |
| | CD3 CAR | DIQMTQTTSSLSASLGDRVTISCRASQDIRNYLNWYQQKPDGTVKL | 126 |
| | without | LIYYTSRLHSGVPSKFSGSGSGTDYSLTISNLEQEDIATYFCQQGNT | |
| | signal | LPWTFAGGTKLEIKGGGGSGGGGSGGGGSGGGGSEVQLQQSGPEL | |
| | peptide | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFSKPTTTPAPRPPTPAPTIASQPLSL | |
| | | RPEACRPAAGGAVHTRGLDFACDIYIWAPLAGTCGVLLLSLVITLY | |
| | | CKRGRKKLLYIFKQPFMRPVQTTQEEDGCCRFPEEEEGGCELRVK | |
| | | FSRSADAPAYQQGQNQLYNELNLGRREEYDVLDKRRGRDPEMGG | |
| | | KPRRKNPQEGLYNELQKDKMAEAYSEIGMKGERRRGKGHDGLYQ | |
| | | GLSTATKDTYDALHMQALPPR | |
| | CD8α | MALPVTALLLPLALLLHAARP | 97 |
| | signal | | |
| | peptide | | |
| | scFv | DIQMTQTTSSLSASLGDRVTISCRASQDIRNYLNWYQQKPDGTVKL | 98 |
| | | LIYYTSRLHSGVPSKFSGSGSGTDYSLTISNLEQEDIATYFCQQGNT | |
| | | LPWTFAGGTKLEIKGGGGSGGGGSGGGGSGGGGSEVQLQQSGPEL | |
| | | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFS | |
| | VH | EVQLQQSGPELVKPGASMKISCKASGYSFTGYTMNWVKQSHGKN | 1 |
| | | LEWMGLINPYKGVSTYNQKFKDKATLTVDKSSSTAYMELLSLTSE | |
| | | DSAVYYCARSGYYGDSDWYFDVWGQGTTLTVFS | |
| | VL | DIQMTQTTSSLSASLGDRVTISCRASQDIRNYLNWYQQKPDGTVKL | 2 |
| | | LIYYTSRLHSGVPSKFSGSGSGTDYSLTISNLEQEDIATYFCQQGNT | |
| | | LPWTFAGGTKLEIK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACD | 101 |
| | hinge and | IYIWAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPE | 103 |
| | signaling | MGGKPRRKNPQEGLYNELQKDKMAEAYSEIGMKGERRRGKGHD | |
| | domain of | GLYQGLSTATKDTYDALHMQALPPR | |
| | CD3ζ | | |
| | IRES | CGGGATCAATTCCGCCCCCCCCCTAACGTTACTGGCCGAAGCCG | 127 |
| | (DNA | CTTGGAATAAGGCCGGTGTGCGTTTGTCTATATGTTATTTTCCAC | |
| | Sequence) | CATATTGCCGTCTTTTGGCAATGTGAGGGCCCGGAAACCTGGCC | |
| | | CTGTCTTCTTGACGAGCATTCCTAGGGGTCTTTCCCCTCTCGCCA | |
| | | AAGGAATGCAAGGTCTGTTGAATGTCGTGAAGGAAGCAGTTCC | |
| | | TCTGGAAGCTTCTTGAAGACAAACAACGTCTGTAGCGACCCTTT | |
| | | GCAGGCAGCGGAACCCCCCACCTGGCGACAGGTGCCTCTGCGG | |
| | | CCAAAAGCCACGTGTATAAGATACACCTGCAAAGGCGGCACAA | |
| | | CCCCAGTGCCACGTTGTGAGTTGGATAGTTGTGGAAAGAGTCAA | |
| | | ATGGCTCTCCTCAAGCGTATTCAACAAGGGGCTGAAGGATGCCC | |
| | | AGAAGGTACCCCATTGTATGGGATCTGATCTGGGGCCTCGGTGC | |
| | | ACATGCTTTACATGTGTTTAGTCGAGGTTAAAAAAACGTCTAGG | |
| | | CCCCCCGAACCACGGGGACGTGGTTTTCCTTTGAAAAACACGAT | |
| | | AATACC | |
| | CD20t* | MTTPRNSVNGTFPAEPMKGPIAMQSGPKPLFRRMSSLVGPTQSFFM | 124 |
| | | RESKTLGAVQIMNGLFHIALGGLLMIPAGIYAPICVTVWYPLWGGI | |
| | | MYIISGSLLAATEKNSRKCLVKGKMIMNSLSLFAAISGMILSIMDIL | |
| | | NIKISHFLKMESLNFIRAHTPYINIYNCEPANPSEKNSPSTQYCYSIQS | |
| | | LFLGILSVMLIFAFFQELVIAGIVENEWKRTCSRPKSNIVLLSAEEKK | |
| | | EQTIEIKEEVVGLTETSSQPKNEEDIE | |
| Anti- | CD20t- | MALPVTALLLPLALLLHAARPMTTPRNSVNGTFPAEPMKGPIAMQ | 128 |
| CD3 | P2A-CD3 | SGPKPLFRRMSSLVGPTQSFFMRESKTLGAVQIMNGLFHIALGGLL | |
| (CD20t- | CAR | MIPAGIYAPICVTVWYPLWGGIMYIISGSLLAATEKNSRKCLVKGK | |
| GSG- | | MIMNSLSLFAAISGMILSIMDILNIKISHFLKMESLNFIRAHTPYINIY | |
| P2A- | | NCEPANPSEKNSPSTQYCYSIQSLFLGILSVMLIFAFFQELVIAGIVE | 129 |
| UCHT1- | | NEWKRTCSRPKSNIVLLSAEEKKEQTIEIKEEVVGLTETSSQPKNEE | |
| CD8 | | DIEGSGATNFSLLKQAGDVEENPGPMALPVTALLLPLALLLHAARP | |
| hinge- | | DIQMTQTTSSLSASLGDRVTISCRASQDIRNYLNWYQQKPDGTVKL | |
| 41BB- | | LIYYTSRLHSGVPSKFSGSGSGTDYSLTISNLEQEDIATYFCQQGNT | |
| CD3zeta) | | LPWTFAGGTKLEIKGGGGSGGGGSGGGGSGGGGSEVQLQQSGPEL | |
| CAR | | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFSKPTTTPAPRPPTPAPTIASQPLSL | |
| | | RPEACRPAAGGAVHTRGLDFACDIYIWAPLAGTCGVLLLSLVITLY | |
| | | CKRGRKKLLYIFKQPFMRPVQTTQEEDGCSCRFPEEEEGGCELRVK | |
| | | FSRSADAPAYQQGQNQLYNELNLGRREEYDVLDKRRGRDPEMGG | |
| | | KPRRKNPQEGLYNELQKDKMAEAYSEIGMKGERRRGKGHDGLYQ | |
| | | GLSTATKDTYDALHMQALPPR | |
| | CD20t- | MTTPRNSVNGTFPAEPMKGPIAMQSGPKPLFRRMSSLVGPTQSFFM | |
| | P2A-CD3 | RESKTLGAVQIMNGLFHIALGGLLMIPAGIYAPICVTVWYPLWGGI | |
| | CAR | MYIISGSLLAATEKNSRKCLVKGKMIMNSLSLFAAISGMILSIMDIL | |
| | without | NIKISHFLKMESLNFIRAHTPYINIYNCEPANPSEKNSPSTQYCYSIQS | |
| | signal | LFLGILSVMLIFAFFQELVIAGIVENEWKRTCSRPKSNIVLLSAEEKK | |
| | peptide | EQTIEIKEEVVGLTETSSQPKNEEDIEGSGATNFSLLKQAGDVEENP | |
| | | GPMALPVTALLLPLALLLHAARPDIQMTQTTSSLSASLGDRVTISCR | |
| | | ASQDIRNYLNWYQQKPDGTVKLLIYYTSRLHSGVPSKFSGSGSGTD | |
| | | YSLTISNLEQEDIATYFCQQGNTLPWTFAGGTKLEIKGGGGSGGGG | |
| | | SGGGGSGGGGSEVQLQQSGPELVKPGASMKISCKASGYSFTGYTM | |
| | | NWVKQSHGKNLEWMGLINPYKGVSTYNQKFKDKATLTVDKSSST | |
| | | AYMELLSLTSEDSAVYYCARSGYYGDSDWYFDVWGQGTTLTVFS | |
| | | KPTTTPAPRPPTPAPTIASQPLSLRPEACRPAAGGAVHTRGLDFACD | |
| | | IYIWAPLAGTCGVLLLSLVITLYCKRGRKKLLYIFKQPFMRPVQTTQ | |
| | | EEDGCSCRFPEEEEGGCELRVKFSRSADAPAYQQGQNQLYNELNL | |
| | | GRREEYDVLDKRRGRDPEMGGKPRRKNPQEGLYNELQKDKMAE | |
| | | AYSEIGMKGERRRGKGHDGLYQGLSTATKDTYDALHMQALPPR | |
| | CD8α | MALPVTALLLPLALLLHAARP | 97 |
| | signal | | |
| | peptide | | |
| | scFV | DIQMTQTTSSLSASLGDRVTISCRASQDIRNYLNWYQQKPDGTVKL | 98 |
| | | LIYYTSRLHSGVPSKFSGSGSGTDYSLTISNLEQEDIATYFCQQGNT | |
| | | LPWTFAGGTKLEIKGGGGSGGGGSGGGGSGGGGSEVQLQQSGPEL | |
| | | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFS | |
| | VH | EVQLQQSGPELVKPGASMKISCKASGYSFTGYTMNWVKQSHGKN | 1 1 |
| | | LEWMGLINPYKGVSTYNQKFKDKATLTVDKSSSTAYMELLSLTSE | |
| | | DSAVYYCARSGYYGDSDWYFDVWGQGTTLTVFS | |
| | VL | DIQMTQTTSSLSASLGDRVTISCRASQDIRNYLNWYQQKPDGTVKL | 2 |
| | | LIYYTSRLHSGVPSKFSGSGSGTDYSLTISNLEQEDIATYFCQQGNT | |
| | | LPWTFAGGTKLEIK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8 | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACD | 101 |
| | hinge and | IYIWAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPE | 103 |
| | signaling | MGGKPRRKNPQEGLYNELQKDKMAEAYSEIGMKGERRRGKGHD | |
| | domain of | GLYQGLSTATKDTYDALHMQALPPR | |
| | CD3ζ | | |
| | GSG-P2A | GSGATNFSLLKQAGDVEENPGP | 122 |
| | CD20t* | MTTPRNSVNGTFPAEPMKGPIAMQSGPKPLFRRMSSLVGPTQSFFM | 124 |
| | | RESKTLGAVQIMNGLFHIALGGLLMIPAGIYAPICVTVWYPLWGGI | |
| | | MYIISGSLLAATEKNSRKCLVKGKMIMNSLSLFAAISGMILSIMDIL | |
| | | NIKISHFLKMESLNFIRAHTPYINIYNCEPANPSEKNSPSTQYCYSIQS | |
| | | LFLGILSVMLIFAFFQELVIAGIVENEWKRTCSRPKSNIVLLSAEEKK | |
| | | EQTIEIKEEVVGLTETSSQPKNEEDIE | |
| Anti- | CD3 CAR | MALPVTALLLPLALLLHAARPGGGGSCPYSNPSLCSGGGGSCPYSN | 130 |
| CD3 | | PSLCSGGGGSCPYSNPSLCGGGGSGGGGSDIQMTQTTSSLSASLGD | |
| (CD20 | | RVTISCRASQDIRNYLNWYQQKPDGTVKLLIYYTSRLHSGVPSKFS | |
| 3x | | GSGSGTDYSLTISNLEQEDIATYFCQQGNTLPWTFAGGTKLEIKGG | |
| mimo- | | GGSGGGGSGGGGSGGGGSEVQLQQSGPELVKPGASMKISCKASGY | |
| linker- | | SFTGYTMNWVKQSHGKNLEWMGLINPYKGVSTYNQKFKDKATLT | |
| UCHT1- | | VDKSSSTAYMELLSLTSEDSAVYYCARSGYYGDSDWYFDVWGQG | |
| CD8 | | TTLTVFSKPTTTPAPRPPTPAPTIASQPLSLRPEACRPAAGGAVHTR | |
| hinge- | | GLDFACDIYIWAPLAGTCGVLLLSLVITLYCKRGRKKLLYIFKQPF | |
| 41bb- | | MRPVQTTQEEDGCSCRFPEEEEGGCELRVKFSRSADAPAYQQGQN | |
| CD3hinge) | | QLYNELNLGRREEYDVLDKRRGRDPEMGGKPRRKNPQEGLYNEL | |
| CAR | | QKDKMAEAYSEIGMKGERRRGKGHDGLYQGLSTATKDTYDALH | |
| | | MQALPPR | |
| | CD3 CAR | GGGGSCPYSNPSLCSGGGGSCPYSNPSLCSGGGGSCPYSNPSLCGG | 131 |
| | without | GGSGGGGSDIQMTQTTSSLSASLGDRVTISCRASQDIRNYLNWYQQ | |
| | signal | KPDGTVKLLIYYTSRLHSGVPSKFSGSGSGTDYSLTISNLEQEDIAT | |
| | peptide | YFCQQGNTLPWTFAGGTKLEIKGGGGSGGGGSGGGGSGGGGSEV | |
| | | QLQQSGPELVKPGASMKISCKASGYSFTGYTMNWVKQSHGKNLE | |
| | | WMGLINPYKGVSTYNQKFKDKATLTVDKSSSTAYMELLSLTSEDS | |
| | | AVYYCARSGYYGDSDWYFDVWGQGTTLTVFSKPTTTPAPRPPTPA | |
| | | PTIASQPLSLRPEACRPAAGGAVHTRGLDFACDIYIWAPLAGTCGV | |
| | | LLLSLVITLYCKRGRKKLLYIFKQPFMRPVQTTQEEDGCSCRFPEEE | |
| | | EGGCELRVKFSRSADAPAYQQGQNQLYNELNLGRREEYDVLDKR | |
| | | RGRDPEMGGKPRRKNPQEGLYNELQKDKMAEAYSEIGMKGERRR | |
| | | GKGHDGLYQGLSTATKDTYDALHMQALPPR | |
| | CD8α | MALPVTALLLPLALLLHAARP | 97 |
| | signal | | |
| | peptide | | |
| | scFV | DIQMTQTTSSLSASLGDRVTISCRASQDIRNYLNWYQQKPDGTVKL | 98 |
| | | LIYYTSRLHSGVPSKFSGSGSGTDYSLTISNLEQEDIATYFCQQGNT | |
| | | LPWTFAGGTKLEIKGGGGSGGGGGGGGSGGGGSEVQLQQSGPEL | |
| | | VKPGASMKISCKASGYSFTGYTMNWVKQSHGKNLEWMGLINPYK | |
| | | GVSTYNQKFKDKATLTVDKSSSTAYMELLSLTSEDSAVYYCARSG | |
| | | YYGDSDWYFDVWGQGTTLTVFS | |
| | VH | EVQLQQSGPELVKPGASMKISCKASGYSFTGYTMNWVKQSHGKN | 1 |
| | | LEWMGLINPYKGVSTYNQKFKDKATLTVDKSSSTAYMELLSLTSE | |
| | | DSAVYYCARSGYYGDSDWYFDVWGQGTTLTVFS | |
| | VL | DIQMTQTTSSLSASLGDRVTISCRASQDIRNYLNWYQQKPDGTVKL | 2 |
| | | LIYYTSRLHSGVPSKFSGSGSGTDYSLTISNLEQEDIATYFCQQGNT | |
| | | LPWTFAGGTKLEIK | |
| | scFv | GGGGSGGGGSGGGGSGGGGS | 85 |
| | Linker | | |
| | CD8α | KPTTTPAPRPPTPAPTIASQPLSLRPEACRPAAGGAVHTRGLDFACD | 99 |
| | hinge | | |
| | CD8α | IYIWAPLAGTCGVLLLSLVITLYC | 100 |
| | transmembrane | | |
| | CD8α | KPTTTPAPRPPTPAPTIASQPLSLRPEACRPAAGGAVHTRGLDFACD | 101 |
| | hinge and | IYIWAPLAGTCGVLLLSLVITLYC | |
| | transmembrane | | |
| | domain | | |
| | Costimulatory | KRGRKKLLYIFKQPFMRPVQTTQEEDGCSCRFPEEEEGGCEL | 102 |
| | domain of | | |
| | 4-1BB | | |
| | Primary | RVKFSRSADAPAYQQGQNQLYNELNLGRREEYDVLDKRRGRDPE | 103 |
| | signaling | MGGKPRRKNPQEGLYNELQKDKMAEAYSEIGMKGERRRGKGHD | |
| | domain of | GLYQGLSTATKDTYDALHMQALPPR | |
| | CD3ζ | | |
| | CD20 3x | GGGGSCPYSNPSLCSGGGGSCPYSNPSLCSGGGGSCPYSNPSLCGGGGSGG | 132 |
| | mimo | GGS | |

In one aspect, the disclosure provides a vector comprising a nucleic acid molecule described herein. In some embodiments the vector comprises a nucleic acid molecule encoding a CAR described herein, e.g., a CD3 CAR. In some embodiments, the vector comprises a nucleic acid molecule encoding a PEBL described herein, e.g., a CD3 PEBL. In one embodiment, the vector is selected from the group consisting of a DNA, a RNA, a plasmid, a lentivirus vector, an adenoviral vector, or a retrovirus vector. In some embodiments, the vector is a murine retroviral vector, e.g., murine stem cell virus (MSCV) retroviral vector. In some embodiments, the vector further comprises a promoter. In some embodiments, the promoter is an EF-1α promoter, a MSCV promoter, SV40 promoter, or a PGK promoter. In some embodiments, the vector further comprises a poly A tail. In some embodiments, the promoter is an EF-1α promoter having a nucleic acid sequence as set forth in SEQ TD NO: 133 or a functional fragment thereof. In some embodiments, the promoter is an MSCV promoter having a nucleic acid sequence as set forth in SEQ TD NO: 134 or a functional fragment thereof. In some embodiments, the disclosure provides two vectors wherein the first vector comprises a nucleic acid molecule encoding a CAR described herein, e.g., a CD3 CAR and the second vector comprises a nucleic acid molecule encoding a PEBL described herein, e.g., a CD3 PEBL.

In some embodiments, the vector may comprise a nucleic acid molecule encoding a CAR described herein, a PEBL described herein, a kill gene described herein, or a combination thereof. A PEBL may be encoded by a first polynucleotide and a CAR may be encoded by a second polynucleotide. In some embodiments, the second polynucleotide may comprise the kill gene. A vector may comprise a first polynucleotide encoding the PEBL and/or a a second polynucleotide encoding the CAR. A vector may comprise a first polynucleotide encoding the PEBL and/or a a second polynucleotide encoding the CAR and the kill gene. In some embodiments, the vector may be a lentiviral vector, a retroviral vector, an adenoviral vector, or an adeno-associated viral (AAV) vector.

TABLE 10
Promoter sequences

| | | SEQ ID |
|---|---|---|
| Description | Sequence | NO: |
| EF-1α | CGTGAGGCTCCGGTGCCCGTCAGTGGGCAGAGCGCACATCGCCCACAGTCC | 133 |
| promoter | CCGAGAAGTTGGGGGGAGGGGTCGGCAATTGAACCGGTGCCTAGAGAAGG | |
| | TGGCGCGGGGTAAACTGGGAAAGTGATGTCGTGTACTGGCTCCGCCTTTTTC | |
| | CCGAGGGTGGGGGAGAACCGTATATAAGTGCAGTAGTCGCCGTGAACGTTC | |
| | TTTTTCGCAACGGGTTTGCCGCCAGAACACAGGTAAGTGCCGTGTGTGGTTC | |
| | CCGCGGGCCTGGCCTCTTTACGGGTTATGGCCCTTGCGTGCCTTGAATTACT | |
| | TCCACCTGGCTGCAGTACGTGATTCTTGATCCCGAGCTTCGGGTTGGAAGTG | |
| | GGTGGGAGAGTTCGAGGCCTTGCGCTTAAGGAGCCCCTTCGCCTCGTGCTTG | |
| | AGTTGAGGCCTGGCCTGGGCGCTGGGGCCGCCGCGTGCGAATCTGGTGGCA | |
| | CCTTCGCGCCTGTCTCGCTGCTTTCGATAAGTCTCTAGCCATTTAAAATTTTT | |
| | GATGACCTGCTGCGACGCTTTTTTTCTGGCAAGATAGTCTTGTAAATGCGGG | |
| | CCAAGATCTGCACACTGGTATTTCGGTTTTTGGGGCCGCGGGCGGCGACGG | |
| | GGCCCGTGCGTCCCAGCGCACATGTTCGGCGAGGCGGGGCCTGCGAGCGCG | |
| | GCCACCGAGAATCGGACGGGGGTAGTCTCAAGCTGGCCGGCCTGCTCTGGT | |
| | GCCTGGCCTCGCGCCGCCGTGTATCGCCCCGCCCTGGGCGGCAAGGCTGGC | |
| | CCGGTCGGCACCAGTTGCGTGAGCGGAAAGATGGCCGCTTCCCGGCCCTGC | |
| | TGCAGGGAGCTCAAAATGGAGGACGCGGCGCTCGGGAGAGCGGGCGGGTG | |
| | AGTCACCCACACAAAGGAAAAGGGCCTTTCCGTCCTCAGCCGTCGCTTCAT | |
| | GTGACTCCACGGAGTACCGGGCGCCGTCCAGGCACCTCGATTAGTTCTCGA | |
| | GCTTTTGGAGTACGTCGTCTTTAGGTTGGGGGGAGGGGTTTTATGCGATGGA | |
| | GTTTCCCCACACTGAGTGGGTGGAGACTGAAGTTAGGCCAGCTTGGCACTT | |
| | GATGTAATTCTCCTTGGAATTTGCCCTTTTTGAGTTTGGATCTTGGTTCATTC | |
| | TCAAGCCTCAGACAGTGGTTCAAAGTTTTTTTCTTCCATTTCAGGTGTCGTG | |
| | A | |
| MSCV | GGAATGAAAGACCCCACCTGTAGGTTTGGCAAGCTAGCTTAAGTAACGCCA | 134 |
| promoter | TTTTGCAAGGCATGGAAAATACATAACTGAGAATAGAGAAGTTCAGATCAA | |
| | GGTTAGGAACAGAGAGACAGCAGAATATGGGCCAAACAGGATATCTGTGG | |
| | TAAGCAGTTCCTGCCCCGGCTCAGGGCCAAGAACAGATGGTCCCCAGATGC | |
| | GGTCCCGCCCTCAGCAGTTTCTAGAGAACCATCAGATGTTTCCAGGGTGCCC | |
| | CAAGGACCTGAAATGACCCTGTGCCTTATTTGAACTAACCAATCAGTTCGCT | |
| | TCTCGCTTCTGTTCGCGCGCTTCTGCTCCCCGAGCTCAATAAAAGAGCCCAC | |
| | AACCCCTCACTCGGCGCGCCAGTCC | |

In one aspect, the disclosure provides an immune cell, e.g., an engineered immune cell comprising a vector described herein. In some embodiments, the disclosure provides an immune cell population, e.g., an engineered immune cell population wherein about 10%, about 20%, about 30%, about 40%, about 50%, about 60%, about 70%, about 80%, about 90%, about 95%, about 99%, about 99.9%, or about 100% of the immune cells of the immune cell population comprises a vector described herein. In some embodiments, the engineered immune cell comprises a nucleic acid molecule described herein, e.g., a nucleic acid molecule encoding a CAR described herein, e.g., a CD3 CAR. In some embodiments, the engineered immune cell comprises a nucleic acid molecule described herein, e.g., a nucleic acid molecule encoding a PEBL described herein, e.g., a CD3 PEBL. In some embodiments, the engineered immune cell comprises a first nucleic acid molecule described herein, e.g., a nucleic acid molecule encoding a CAR described herein, e.g., a CD3 CAR and a second nucleic acid molecule described herein, e.g., a nucleic acid molecule encoding a PEBL described herein, e.g., a CD3 PEBL. In some embodiments, the engineered immune cell is an engineered T cell, an engineered natural killer (NK) cell, an engineered NK/T cell, an engineered monocyte, an engineered macrophage, or an engineered dendritic cell. In some embodiments, the engineered immune cell is an engineered T cell. In some embodiments, the immune cell is a peripheral blood mononuclear cell, e.g., a T cell. In some embodiments, at least 10%, at least 20%, at least 30%, at least 40%, at least 50%, at least 60%, at least 70%, at least 80%, at least 90%, or about 100% of the cells of the immune cell population are CD4-positive T cells. In some embodiments, at least 10%, at least 20%, at least 30%, at least 40%, at least 50%, at least 60%, at least 70%, at least 80%, at least 90%, or about 100% of the cells of the immune cell population are CD8-positive T cells.

Nucleic acid includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In certain embodiments, nucleic acid molecules can be modified. In the case of a double-stranded polymer, “nucleic acid” can refer to either or both strands of the molecule.

As will be appreciated by those of skill in the art, in some aspects, the nucleic acid further comprises a plasmid sequence. The plasmid sequence can include, for example, one or more sequences of a promoter sequence, a selection marker sequence, or a locus-targeting sequence.

As those skilled in the art would appreciate, in certain embodiments, any of the sequences of the various components disclosed herein (e.g., scFv, intracellular signaling domain, hinge, linker, localizing sequences, and combinations thereof) can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the specific corresponding sequences disclosed herein. For example, in certain embodiments, the intracellular signaling domain 4-1BB can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to SEQ ID NO: 154, as long as it possesses the desired function.

In certain aspects of the present disclosure, the chimeric antigen receptor (CAR) can bind to a molecule that is expressed on the surface of a cell including, but not limited to members of the CD1 family of glycoproteins, CD2, CD3, CD4, CD5, CD7, CD3, CD25, CD28, CD30, CD38, CD45, CD45RA, CD45RO, CD52, CD56, CD57, CD99, CD127, and CD137.

As described herein, in some embodiments, T cell health markedly increased when a CD3 CAR was used in combination with downregulation of CD3 expression on the effector T cells. In some embodiments, downregulation (e.g., elimination, reduction, and/or relocalization) of CD3 prevented the fratricidal effect exerted by the corresponding CD3 CAR, allowing greater T cell recovery after CAR expression as compared to cells that retained the target antigen (e.g., CD3), and a more effective cytotoxicity against T leukemia/lymphoma cells. As those of skill in the art would appreciate, downregulation of CD3 expression on the effector T cells can be achieved according to a variety of known methods including, for example, “intrabodies” against CD3, RNAi against CD3, or gene editing methods such as, e.g., meganucleases, TALEN, CRISPR/Cas9, and zinc finger nucleases.

In some embodiments, the engineered immune cell further comprises a nucleic acid that comprises a nucleotide sequence encoding a target-binding molecule linked to a localizing domain. The “target-binding molecule linked to a localizing domain” is sometimes referred to herein as a protein expression blocker (PEBL) or in some cases, an “intrabody”, as described in WO2016/126213, the teachings of which are incorporated by reference in their entirety. Exemplary embodiments of a PEBL are shown in Table 12. In some embodiment, the target binding molecule further comprises a leader sequence, e.g., a leader sequence described herein. In some embodiments, the leader sequence encodes a CD8a signal peptide. In some embodiments, the leader sequence comprises an amino acid sequence of SEQ ID NO: 97, or a sequence with 95-99% identity to an amino acid sequence of SEQ ID NO: 97.

As used herein, “linked” in the context of the protein expression blocker refers to a nucleic acid sequence encoding a target-binding domain directly in frame (e.g., without a linker) adjacent to one or more nucleic acid sequences encoding one or more localizing domains. Alternatively, the nucleic acid sequence encoding a target-binding domain may be connected to one or more nucleic acid sequences encoding one or more localizing domains through a linker sequence, e.g., as described in WO2016/126213. In some embodiments, the linker comprises an amino acid sequence as set forth in SEQ ID NO: 135 (GGGGSGGGGSGGGGSGGGGSAE). Non-limiting examples of a linker include (GS)n (SEQ ID NO: 87), (GGS)n (SEQ ID NO: 88), (Gly3Ser)n (SEQ ID NO: 89), (Gly2SerGly)n (SEQ ID NO: 90), (Gly2SerGly2)n (SEQ ID NO: 91), or (Gly4Ser)n (SEQ ID NO: 92), wherein n is 1, 2, 3, 4, 5, 6, 7, 8, 9, or 10. In some embodiments, the linker is (GGGGS)n where n can range from 1 to 6, e.g., 1, 2, 3, 4, 5, or 6 (SEQ ID NO: 86). In some embodiments, the linker is (GGGGS)n wherein n is an integer of, e.g., 2-12 (SEQ ID NO: 136). In some embodiment, the linker is (Gly4Ser)3 (SEQ ID NO: 93) or (Gly4Ser)4 (SEQ ID NO: 85). In some embodiments, the linker may comprise an amino acid sequence of GGGGS GGGGS GGGGS (SEQ ID NO: 93). In some embodiments, the linker may comprise an amino acid sequence of GGGGSGGGGS (SEQ ID NO: 94)

In some embodiments, the nucleic acid sequence encoding a target-binding domain may be connected to one or more nucleic acid sequences encoding a localizing domain, e.g., a localizing domain tethered to the target-binding molecule with a myc sequence.

In some embodiments, the PEBL molecules of the disclosed herein can comprise one or more localizing domains, e.g., connected by an intervening linker. When more than one localizing domain is used in a given PEBL molecule, each localizing domain can be linked with or without any intervening linker. In some instances, localizing domains such as a CD8a transmembrane domain, KDEL motif (SEQ ID NO: 137), and a linker can be used in a single PEBL molecule.

In some embodiments, a percentage of the engineered immune cells of the cell population may have a reduced expression of a cell surface marker (e.g., CD3) compared to that of an otherwise identical cell population that does not comprise an immune cell comprising a PEBL. In some embodiments, at least about 5%, at least about 10%, at least about 20%, at least about 30%, at least 40%, at least about 50%, at least about 60%, at least about 70%, at least about 80%, at least about 90%, or at least about 100% of the engineered immune cells of the cell population may have a reduced expression of a cell surface marker (e.g., CD3) compared to that of an otherwise identical cell population that does not comprise an immune cell comprising a PEBL. The cell population may have at least about a 5-fold, at least about a 10-fold, at least about a 20-fold, at least about a 25-fold, at least about a 30-fold, at least about a 40-fold, or at least about a 50-fold reduction of a cell surface marker (e.g., CD3) compared to that of an otherwise identical cell population that does not comprise an immune cell comprising a PEBL.

In some embodiments, the engineered immune cells or cells of the cell population express a CAR as described herein. In some embodiments, at least about 5%, at least about 10%, at least about 20%, at least about 30%, at least about 40%, at least about 50%, at least about 60%, at least about 70%, at least about 80%, at least about 90%, or at least about 100% of the engineered immune cells of the cell population express the CAR. In some embodiments, at most about 100%, at most about 90%, at most about 80%, at most about 70%, at most about 60%, at most about 50%, at most about 40%, at most about 30%, at most about 20%, at most about 10%, or at most about 5% of the engineered immune cells of the cell population express the CAR. In some embodiments, from about 1% to about 100% of the engineered immune cells of the cell population express the CAR. In some embodiments, from about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% of the engineered immune cells of the cell population express the CAR.

The cell population can be capable of expansion over a period of time. In some embodiments, the cell population as described herein can be capable of at least about at least about a 5-fold, at least about a 10-fold, at least about a 20-fold, at least about a 25-fold, at least about a 30-fold, at least about a 40-fold, or at least about a 50-fold expansion in a number of days (e.g., 1 day, 2 days, 3 days, 4 days, 5 days, 10 days, 15 days, 20 days, or 30 days).

In some embodiments, the target binding molecule comprises at least one, at least two, at least three, at least four, or at least five target binding domains. In some embodiments, the target binding molecule comprises one target binding domain, e.g., a CD3 binding domain. In some embodiments, the target binding molecule comprises a first target binding domain, e.g., a first CD3 binding domain, and a second target binding domain, e.g., a second CD3 binding domain. In some embodiments, the first target binding domain and the second target binding domain are identical. In some embodiments, the first target binding domain and the second target binding domain are different. In some embodiments, the first target binding domain and the second target binding domain are connected via a linker, e.g., a peptide linker. In some embodiments, the linker comprises an amino acid sequence as set forth in SEQ ID NO: 135.

In some embodiments, the antibody or the antigen binding fragment that binds CD3 in the context of the CAR, e.g., CD3 CAR, as described herein, can be different from the antibody or the antigen binding fragment that binds CD3 in the context of the PEBL, e.g., CD3 PEBL. In some embodiments, the antibody or the antigen binding fragment that binds CD3 in the context of the CAR, as described herein, can be the same as the antibody or the antigen binding fragment that binds CD3 in the context of the PEBL. In some embodiments, the antibody or the antigen binding fragment that binds CD3 in the context of the CAR, as described herein, can have at least 90%, 91%, 92%, 93%, 94%, 95%, 96%, 97%, 98%, or 99% sequence identity to the antibody or the antigen binding fragment that binds CD3 in the context of the PEBL.

In some embodiments, the localizing domain of the PEBL comprises one or more of an ER retention sequence, a Golgi retention sequence; a proteosome localizing sequence; or a transmembrane domain sequence derived from, 4-1BB, CD28, CD34, CD4, CD8, FcεRIγ, CD16, OX40, CD3ζ, CD3ε, CD3γ, CD3δ, TCRα, TCRβ, CD32, CD64, VEGFR2, FAS, or FGFR2B.

In some embodiments, the localizing domain comprises an ER retention sequence comprising a sequence of KDEL (SEQ ID NO:137). In some embodiments, the ER retention sequence is fused a binding domain, e.g., a CD3 binding domain, via a linker peptide. In some embodiments, the linker peptide comprises a MYC tag (SEQ ID NO: 139). In some embodiments the linker and the ER retention sequence together have a sequence as set forth in SEQ ID NO: 141. In some embodiments, the linker peptide comprises one or more (e.g., 1, 2, 3, 4, 5, 6, or 7) GGGGS sequences (SEQ ID NO: 138). In some embodiments, the ER retention sequence and linker together comprise a sequence as set forth in SEQ ID NO: 143. In some embodiments, the intracellular targeting signal comprises an ER retention sequence comprising an amino acid sequence as set forth in any one of SEQ ID NOs: 137-143. Table 11 provides the amino acid and nucleic acid sequences of some exemplary ER retention peptides and intracellular targeting signal components.

TABLE 11
Amino acid sequences of exemplary ER retention peptides and intracellular
targeting signal components

| | | SEQ ID |
|---|---|---|
| Description | SEQUENCE | NO: |
| Myc Tag | EQKLISEEDL | 139 |
| ER retention sequence | KDEL | 137 |
| ER retention sequence | AEKDEL | 140 |
| ER retention sequence with a Myc | EQKLISEEDLKDEL | 141 |
| linker (“myc KDEL”) | | |
| ER retention sequence | GGGGSGGGGSGGGGSGGGGSAEKDEL | 142 |
| ER retention sequence | EQKLISEEDLGGGGSGGGGSGGGGSGGG | 143 |
| | GSAEKDEL | |
| ER retention peptide AEKDEL | DDHYCLDYWGQGTTLTVSSAAEKDEL | 144 |
| ER retention peptide EEKKMP | KYKSRRSFIEEKKMP | 145 |
| Localizing domain “mb DEKKMP” | TTTPAPRPPTPAPTIASQPLSLRPEACRPA | 146 |
| | AGGAVHTRGLDFACDIYIWAPLAGTCG | |
| | VLLLSLVITLYKYKSRRSFIDEKKMP | |
| Golgi retention sequence | YQRL | 147 |
| Golgi retention sequence | YGRL | 148 |
| Golgi retention sequence | YKGL | 149 |
| Proteasome localizing sequence | PEST | 150 |
| PEST motif | SHGFPPEVEEQDDGTLPMSCAQESGMDR | 151 |
| | HPAACASARINV | |
| The ER or Golgi retention sequence | KKXX where X is any amino acid | N/A |
| The ER or Golgi retention sequence | KXD/E (such as KXD or KXE) where X is | N/A |
| | any amino acid | |
| The ER or Golgi retention sequence | YXXL where X is any amino acid | N/A |

The intracellular targeting signal can direct the PEBL to a specific cellular compartment, such as the Golgi or endoplasmic reticulum (ER), the proteasome, or the cell membrane, depending on the application. In some embodiments, the ER or Golgi retention sequence comprises the amino acid sequence selected from KDEL (SEQ TD NO: 137), YQRL (SEQ TD NO: 147), YGRL (SEQ TD NO: 148), YKGL (SEQ ID NO: 149), KKXX where X is any amino acid (SEQ Identifier A1), KXD/E (such as KXD or KXE) where X is any amino acid (SEQ Identifier A2), or YXXL where X is any amino acid (SEQ Identifier A3). In some embodiments, the Golgi retention sequence is any one of SEQ TD NOs: 147-149. In some embodiments, the proteosome localizing sequence comprises an amino acid sequence as set forth in SEQ TD NO: 150). In some embodiments, the proteosome localizing sequence can comprise a PEST motif as set forth in SEQ TD NO: 151.

In certain embodiments, the protein expression blocker (PEBL) can have a similar structure as any one or more of the CD3 PEBL as disclosed in WO2016/126213, the disclosure is herein incorporated by reference in its entirety for all purposes. Accordingly, the engineered immune cells described herein can comprise a PEBL (a target-binding molecule linked to a localizing domain) that binds to CD3, as described in WO2016/126213. In certain embodiments, the PEBL may comprise a target binding domain targeting a TCR. In some embodiments, the CD3 PEBL comprises an amino acid sequence as set forth in Table 12, e.g., any of the SEQ ID NOs: 152-159. Table 12 shows amino acid sequences of exemplary PEBLs and selected components.

TABLE 12
Amino acid sequences of exemplary PEBLs and selected components.

| | | | SEQ ID |
|---|---|---|---|
| PEBL | Description | Sequence | NO: |
| UCHT1- | UCHT1 | MALPVTALLLPLALLLHAARPDIQMTQTTSSLSASLGDRVTISCRAS | 152 |
| PEBL | PEBL | QDIRNYLNWYQQKPDGTVKLLIYYTSRLHSGVPSKFSGSGSGTDYS | |
| | | LTISNLEQEDIATYFCQQGNTLPWTFAGGTKLEIKGGGGSGGGGSG | |
| | | GGGSGGGGSEVQLQQSGPELVKPGASMKISCKASGYSFTGYTMNW | |
| | | VKQSHGKNLEWMGLINPYKGVSTYNQKFKDKATLTVDKSSSTAY | |
| | | MELLSLTSEDSAVYYCARSGYYGDSDWYFDVWGQGTTLTVFSEQ | |
| | | KLISEEDLGGGGSGGGGSGGGGSGGGGSAEKDEL | |
| | UCHT1 | DIQMTQTTSSLSASLGDRVTISCRASQDIRNYLNWYQQKPDGTVKL | 153 |
| | PEBL | LIYYTSRLHSGVPSKFSGSGSGTDYSLTISNLEQEDIATYFCQQGNTL | |
| | without | PWTFAGGTKLEIKGGGGSGGGGSGGGGSGGGGSEVQLQQSGPELV | |
| | signal | KPGASMKISCKASGYSFTGYTMNWVKQSHGKNLEWMGLINPYKG | |
| | peptide | VSTYNQKFKDKATLTVDKSSSTAYMELLSLTSEDSAVYYCARSGY | |
| | | YGDSDWYFDVWGQGTTLTVFSEQKLISEEDLGGGGSGGGGSGGGG | |
| | | SGGGGSAEKDEL | |
| | CD8α signal | MALPVTALLLPLALLLHAARP | 97 |
| | peptide | | |
| | UCHT1 | DIQMTQTTSSLSASLGDRVTISCRASQDIRNYLNWYQQKPDGTVKL | 98 |
| | scFv | LIYYTSRLHSGVPSKFSGSGSGTDYSLTISNLEQEDIATYFCQQGNTL | |
| | | PWTFAGGTKLEIKGGGGSGGGGSGGGGSGGGGSEVQLQQSGPELV | |
| | | KPGASMKISCKASGYSFTGYTMNWVKQSHGKNLEWMGLINPYKG | |
| | | VSTYNQKFKDKATLTVDKSSSTAYMELLSLTSEDSAVYYCARSGY | |
| | | YGDSDWYFDVWGQGTTLTVFS | |
| | UCHT1 VH | EVQLQQSGPELVKPGASMKISCKASGYSFTGYTMNWVKQSHGKNL | 1 |
| | | EWMGLINPYKGVSTYNQKFKDKATLTVDKSSSTAYMELLSLTSED | |
| | | SAVYYCARSGYYGDSDWYFDVWGQGTTLTVFS | |
| | UCHT1 VL | DIQMTQTTSSLSASLGDRVTISCRASQDIRNYLNWYQQKPDGTVKL | 2 |
| | | LIYYTSRLHSGVPSKFSGSGSGTDYSLTISNLEQEDIATYFCQQGNTL | |
| | | PWTFAGGTKLEIK | |
| | VH-VL | GGGGSGGGGSGGGGSGGGGS | 85 |
| | linker | | |
| | myc tag | EQKLISEEDL | 139 |
| | GS linker + 2 aa | GGGGSGGGGSGGGGSGGGGSAE | 135 |
| | ER retention | KDEL | 137 |
| | sequence | | |
| | ER retention | EQKLISEEDLGGGGSGGGGSGGGGSGGGGSAEKDEL | 143 |
| | region | | |
| OKT3- | OKT3- | MALPVTALLLPLALLLHAARPQIVLTQSPAIMSASPGEKVTMTCSAS | 154 |
| PEBL | PEBL | SSVSYMNWYQQKSGTSPKRWIYDTSKLASGVPAHFRGSGSGTSYS | |
| | | LTISGMEAEDAATYYCQQWSSNPFTFGSGTKLEINRGGGGSGGGGS | |
| | | GGGGSGGGGSEVQLQQSGAELARPGASVKMSCKASGYTFTRYTM | |
| | | HWVKQRPGQGLEWIGYINPSRGYTNYNQKFKDKATLTTDKSSSTA | |
| | | YMQLSSLTSEDSAVYYCARYYDDHYCLDYWGQGTTLTVSSAEQK | |
| | | LISEEDLGGGGSGGGGSGGGGSGGGGSAEKDEL | |
| | OKT3- | QIVLTQSPAIMSASPGEKVTMTCSASSSVSYMNWYQQKSGTSPKR | 155 |
| | PEBL | WIYDTSKLASGVPAHFRGSGSGTSYSLTISGMEAEDAATYYCQQWS | |
| | without | SNPFTFGSGTKLEINRGGGGSGGGGSGGGGSGGGGSEVQLQQSGAE | |
| | signal | LARPGASVKMSCKASGYTFTRYTMHWVKQRPGQGLEWIGYINPSR | |
| | peptide | GYTNYNQKFKDKATLTTDKSSSTAYMQLSSLTSEDSAVYYCARYY | |
| | | DDHYCLDYWGQGTTLTVSSAEQKLISEEDLGGGGSGGGGSGGGGS | |
| | | GGGGSAEKDEL | |
| | CD8α signal | MALPVTALLLPLALLLHAARP | 97 |
| | peptide | | |
| | OKT3 scFv | QIVLTQSPAIMSASPGEKVTMTCSASSSVSYMNWYQQKSGTSPKR | 106 |
| | | WIYDTSKLASGVPAHFRGSGSGTSYSLTISGMEAEDAATYYCQQWS | |
| | | SNPFTFGSGTKLEINRGGGGSGGGGSGGGGSGGGGSEVQLQQSGAE | |
| | | LARPGASVKMSCKASGYTFTRYTMHWVKQRPGQGLEWIGYINPSR | |
| | | GYTNYNQKFKDKATLTTDKSSSTAYMQLSSLTSEDSAVYYCARYY | |
| | | DDHYCLDYWGQGTTLTVSSA | |
| | OKT3 VH | EVQLQQSGAELARPGASVKMSCKASGYTFTRYTMHWVKQRPGQG | 3 |
| | | LEWIGYINPSRGYTNYNQKFKDKATLTTDKSSSTAYMQLSSLTSED | |
| | | SAVYYCARYYDDHYCLDYWGQGTTLTVSSA | |
| | OKT3 VL | QIVLTQSPAIMSASPGEKVTMTCSASSSVSYMNWYQQKSGTSPKR | 4 |
| | | WIYDTSKLASGVPAHFRGSGSGTSYSLTISGMEAEDAATYYCQQWS | |
| | | SNPFTFGSGTKLEINR | |
| | VH-VL | GGGGSGGGGSGGGGSGGGGS | 85 |
| | linker | | |
| | myc tag | EQKLISEEDL | 139 |
| | GS linker + 2 aa | GGGGSGGGGSGGGGSGGGGSAE | 135 |
| | ER retention | KDEL | 137 |
| | sequence | | |
| | ER retention | EQKLISEEDLGGGGSGGGGSGGGGSGGGGSAEKDEL | 143 |
| | region | | |
| 28F11- | 28F11- | MALPVTALLLPLALLLHAARPEIVLTQSPATLSLSPGERATLSCRAS | 156 |
| PEBL | PEBL | QSVSSYLAWYQQKPGQAPRLLIYDASNRATGIPARFSGSGSGTDFT | |
| | | LTISSLEPEDFAVYYCQQRSNWPPLTFGGGTKVEIKGGGGSGGGGS | |
| | | GGGGSGGGGSQVQLVESGGGVVQPGRSLRLSCAASGFKFSGYGMH | |
| | | WVRQAPGKGLEWVAVIWYDGSKKYYVDSVKGRFTISRDNSKNTL | |
| | | YLQMNSLRAEDTAVYYCARQMGYWHFDLWGRGTLVTVSSEQKLI | |
| | | SEEDLGGGGSGGGGSGGGGSGGGGSAEKDEL | |
| | 28F11- | EIVLTQSPATLSLSPGERATLSCRASQSVSSYLAWYQQKPGQAPRLL | 157 |
| | PEBL | IYDASNRATGIPARFSGSGSGTDFTLTISSLEPEDFAVYYCQQRSNW | |
| | without | PPLTFGGGTKVEIKGGGGSGGGGSGGGGSGGGGSQVQLVESGGGV | |
| | signal | VQPGRSLRLSCAASGFKFSGYGMHWVRQAPGKGLEWVAVIWYDG | |
| | peptide | SKKYYVDSVKGRFTISRDNSKNTLYLQMNSLRAEDTAVYYCARQM | |
| | | GYWHFDLWGRGTLVTVSSEQKLISEEDLGGGGSGGGGSGGGGSGG | |
| | | GGSAEKDEL | |
| | CD8α signal | MALPVTALLLPLALLLHAARP | 97 |
| | peptide | | |
| | 28F11 scFv | EIVLTQSPATLSLSPGERATLSCRASQSVSSYLAWYQQKPGQAPRLL | 109 |
| | | IYDASNRATGIPARFSGSGSGTDFTLTISSLEPEDFAVYYCQQRSNW | |
| | | PPLTFGGGTKVEIKGGGGSGGGGSGGGGSGGGGSQVQLVESGGGV | |
| | | VQPGRSLRLSCAASGFKFSGYGMHWVRQAPGKGLEWVAVIWYDG | |
| | | SKKYYVDSVKGRFTISRDNSKNTLYLQMNSLRAEDTAVYYCARQM | |
| | | GYWHFDLWGRGTLVTVSS | |
| | 28F11 VH | QVQLVESGGGVVQPGRSLRLSCAASGFKFSGYGMHWVRQAPGKG | 5 |
| | | LEWVAVIWYDGSKKYYVDSVKGRFTISRDNSKNTLYLQMNSLRAE | |
| | | DTAVYYCARQMGYWHFDLWGRGTLVTVSS | |
| | 28F11 VL | EIVLTQSPATLSLSPGERATLSCRASQSVSSYLAWYQQKPGQAPRLL | 6 |
| | | IYDASNRATGIPARFSGSGSGTDFTLTISSLEPEDFAVYYCQQRSNW | |
| | | PPLTFGGGTKVEIK | |
| | VH-VL | GGGGSGGGGSGGGGSGGGGS | 85 |
| | linker | | |
| | myc tag | EQKLISEEDL | 139 |
| | GS | GGGGSGGGGSGGGGSGGGGSAE | 135 |
| | linker + 2 aa | | |
| | ER retention | KDEL | 137 |
| | sequence | | |
| | ER retention | EQKLISEEDLGGGGSGGGGSGGGGSGGGGSAEKDEL | 143 |
| | region | | |
| BMA031- | BMA031 | MALPVTALLLPLALLLHAARPQIVLTQSPAIMSASPGEKVTMTCSAT | 158 |
| PEBL | PEBL | SSVSYMHWYQQKSGTSPKRWIYDTSKLASGVPARFSGSGSGTSYSL | |
| | | TISSMEAEDAATYYCQQWSSNPLTFGAGTKLELKGGGGSGGGGSG | |
| | | GGGSGGGGSEVQLQQSGPELVKPGASVKMSCKASGYKFTSYVMH | |
| | | WVKQKPGQGLEWIGYINPYNDVTKYNEKFKGKATLTSDKSSSTAY | |
| | | MELSSLTSEDSAVHYCARGSYYDYDGFVYWGQGTLVTVSAEQKLI | |
| | | SEEDLGGGGSGGGGSGGGGSGGGGSAEKDEL | |
| | BMA031 | QIVLTQSPAIMSASPGEKVTMTCSATSSVSYMHWYQQKSGTSPKR | 159 |
| | PEBL | WIYDTSKLASGVPARFSGSGSGTSYSLTISSMEAEDAATYYCQQWS | |
| | without | SNPLTFGAGTKLELKGGGGSGGGGGGGGSGGGGSEVQLQQSGPE | |
| | signal | LVKPGASVKMSCKASGYKFTSYVMHWVKQKPGQGLEWIGYINPY | |
| | peptide | NDVTKYNEKFKGKATLTSDKSSSTAYMELSSLTSEDSAVHYCARGS | |
| | | YYDYDGFVYWGQGTLVTVSAEQKLISEEDLGGGGSGGGGGGGG | |
| | | SGGGGSAEKDEL | |
| | CD8α signal | MALPVTALLLPLALLLHAARP | 97 |
| | peptide | | |
| | BMA031 | QIVLTQSPAIMSASPGEKVTMTCSATSSVSYMHWYQQKSGTSPKR | 112 |
| | scFv | WIYDTSKLASGVPARFSGSGSGTSYSLTISSMEAEDAATYYCQQWS | |
| | | SNPLTFGAGTKLELKGGGGSGGGGSGGGGSGGGGSEVQLQQSGPE | |
| | | LVKPGASVKMSCKASGYKFTSYVMHWVKQKPGQGLEWIGYINPY | |
| | | NDVTKYNEKFKGKATLTSDKSSSTAYMELSSLTSEDSAVHYCARGS | |
| | | YYDYDGFVYWGQGTLVTVSA | |
| | BMA031 | EVQLQQSGPELVKPGASVKMSCKASGYKFTSYVMHWVKQKPGQG | 7 |
| | VH | LEWIGYINPYNDVTKYNEKFKGKATLTSDKSSSTAYMELSSLTSED | |
| | | SAVHYCARGSYYDYDGFVYWGQGTLVTVSA | |
| | BMA031 | QIVLTQSPAIMSASPGEKVTMTCSATSSVSYMHWYQQKSGTSPKR | 8 |
| | VL | WIYDTSKLASGVPARFSGSGSGTSYSLTISSMEAEDAATYYCQQWS | |
| | | SNPLTFGAGTKLELK | |
| | VH-VL | GGGGSGGGGSGGGGSGGGGS | 85 |
| | linker | | |
| | myc tag | EQKLISEEDL | 139 |
| | GS linker + 2 aa | GGGGSGGGGSGGGGSGGGGSAE | 135 |
| | ER retention | KDEL | 137 |
| | sequence | | |
| | ER retention | EQKLISEEDLGGGGSGGGGSGGGGSGGGGSAEKDEL | 143 |
| | region | | |
| CIV3- | CIV3- | MALPVTALLLPLALLLHAARPDIQMTQSPSTLSASVGDRVTMTCSA | 160 |
| PEBL | PEBL | TSSVSYMHWYQQKPGKAPKRWIYDTSKLASGVPARFIGSGSGTEFT | |
| | | LTISSLQPDDFATYYCQQWSSNPLTFGGGTKVEIKGGGGSGGGGSG | |
| | | GGGSGGGGSQVQLVQSGAEVKKPGSSVKVSCKASGYKFTSYVMH | |
| | | WVKQAPGQGLEWIGYINPYNDVTKYNEKFKGKATLTADESTNTAY | |
| | | MELSSLRSEDTAVHYCARGSYYDYDGFVYWGQGTLVTVSSEQKLI | |
| | | SEEDLGGGGSGGGGSGGGGSGGGGSAEKDEL | |
| | CIV3- | DIQMTQSPSTLSASVGDRVTMTCSATSSVSYMHWYQQKPGKAPKR | 161 |
| | PEBL | WIYDTSKLASGVPARFIGSGSGTEFTLTISSLQPDDFATYYCQQWSS | |
| | without | NPLTFGGGTKVEIKGGGGSGGGGSGGGGSGGGGSQVQLVQSGAEV | |
| | signal | KKPGSSVKVSCKASGYKFTSYVMHWVKQAPGQGLEWIGYINPYN | |
| | peptide | DVTKYNEKFKGKATLTADESTNTAYMELSSLRSEDTAVHYCARGS | |
| | | YYDYDGFVYWGQGTLVTVSSEQKLISEEDLGGGGSGGGGSGGGGS | |
| | | GGGGSAEKDEL | |
| | CD8α signal | MALPVTALLLPLALLLHAARP | 97 |
| | peptide | | |
| | CIV3 scFv | DIQMTQSPSTLSASVGDRVTMTCSATSSVSYMHWYQQKPGKAPKR | 115 |
| | | WIYDTSKLASGVPARFIGSGSGTEFTLTISSLQPDDFATYYCQQWSS | |
| | | NPLTFGGGTKVEIKGGGGSGGGGSGGGGSGGGGSQVQLVQSGAEV | |
| | | KKPGSSVKVSCKASGYKFTSYVMHWVKQAPGQGLEWIGYINPYN | |
| | | DVTKYNEKFKGKATLTADESTNTAYMELSSLRSEDTAVHYCARGS | |
| | | YYDYDGFVYWGQGTLVTVSS | |
| | CIV3 VH | QVQLVQSGAEVKKPGSSVKVSCKASGYKFTSYVMHWVKQAPGQG | 9 |
| | | LEWIGYINPYNDVTKYNEKFKGKATLTADESTNTAYMELSSLRSED | |
| | | TAVHYCARGSYYDYDGFVYWGQGTLVTVSS | |
| | CIV3 VL | DIQMTQSPSTLSASVGDRVTMTCSATSSVSYMHWYQQKPGKAPKR | 10 |
| | | WIYDTSKLASGVPARFIGSGSGTEFTLTISSLQPDDFATYYCQQWSS | |
| | | NPLTFGGGTKVEIK | |
| | VH-VL | GGGGSGGGGSGGGGSGGGGS | 85 |
| | linker | | |
| | myc tag | EQKLISEEDL | 139 |
| | GS linker + 2 aa | GGGGSGGGGSGGGGSGGGGSAE | 135 |
| | ER retention | KDEL | 137 |
| | sequence | | |
| | ER retention | EQKLISEEDLGGGGSGGGGSGGGGGGGGSAEKDEL | 143 |
| | region | | |

In certain aspects of the present disclosure, the protein expression blocker (PEBL) can bind to a molecule that is expressed on the surface of a cell including, but not limited to members of the CD family of glycoproteins, CD2, CD3, CD4, CD5, CD7, CD3, CD25, CD28, CD30, CD38, CD45, CD45RA, CD45RO, CD52, CD56, CD57, CD99, CD127, and CD137.

In some aspects of the present disclosure, expression of a member of the CD family of glycoproteins, CD2, CD3, CD4, CD5, CD7, CD3, CD25, CD28, CD30, CD38, CD45, CD45RA, CD45RO, CD52, CD56, CD57, CD99, CD127, or CD137 can be downregulated using a gene editing method, such as, but not limited to, a gene editing technology that employs meganucleases, TALEN, CRISPR/Cas9, or zinc finger nucleases. For example, in some embodiments, CD3 expression is knocked out using genome editing by Cas9/CRISPR. In other embodiments, CD5 expression is knocked out using genome editing by Cas9/CRISPR. In other embodiments, CD7 expression is knocked out using genome editing by Cas9/CRISPR.

As noted above, downregulation of CD3 expression on the effector T cells can be achieved according to a variety of other known methods including, for example, gene editing methods with meganucleases, TALEN, CRISPR/Cas9, and zinc finger nucleases. Thus, in certain embodiments, the engineered immune cell further comprises a modified CD3 gene, which modification renders the CD3 gene or protein non-functional. By way of example, the engineered immune cell of the present disclosure further comprises a modified (e.g., non-functional) CD3 gene (modified using, e.g., meganucleases, TALEN, CRISPR/Cas9, or zinc finger nucleases) that prevents or reduces expression of CD3, and/or otherwise impairs (e.g., structurally) the CD3 protein from being recognized by a CD3 CAR. Methods of modifying gene expression using such methods are readily available and well-known in the art.

Methods of inactivating a target gene in an immune cell using CRISPR/Cas9 technology are described, for example, in US Patent Publication Nos. 2016/0272999, 2017/0204372, and 2017/0119820.

The CRISPR/Cas system is a system for inducing targeted genetic alterations (genome modifications). Target recognition by the Cas9 protein requires a “seed” sequence within the guide RNA (gRNA) and a conserved multinucleotide containing protospacer adjacent motif (PAM) sequence upstream of the gRNA-binding region. The CRISPR/Cas system can thereby be engineered to cleave substantially any DNA sequence by redesigning the gRNA in cell lines, primary cells, and engineered cells. The CRISPR/Cas system can simultaneously target multiple genomic loci by co-expressing a single Cas9 protein with two or more gRNAs, making this system uniquely suited for multiple gene editing or synergistic activation of target genes. Examples of a CRISPR/Cas system used to inhibit gene expression are described in U.S. Publication No. 2014/0068797 and U.S. Pat. Nos. 8,697,359 and 8,771,945. The system induces permanent gene disruption that utilizes the RNA-guided Cas9 endonuclease to introduce DNA double stranded breaks which trigger error-prone repair pathways to result in frame shift mutations. In some cases, other endonucleases may also be used, including but not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, T7, Fok1, other nucleases known in the art, homologs thereof, or modified versions thereof.

CRISPR/Cas gene disruption occurs when a gRNA sequence specific for a target gene and a Cas endonuclease are introduced into a cell and form a complex that enables the Cas endonuclease to introduce a double strand break at the target gene. In some instances, the CRISPR system comprises one or more expression vectors comprising a nucleic acid sequence encoding the Cas endonuclease and a guide nucleic acid sequence specific for the target gene. The guide nucleic acid sequence is specific for a gene and targets that gene for Cas endonuclease-induced double strand breaks. The sequence of the guide nucleic acid sequence may be within a locus of the gene. In some embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40, 45, 50, or more nucleotides in length. The guide nucleic acid sequence includes an RNA sequence, a DNA sequence, a combination thereof (an RNA-DNA combination sequence), or a sequence with synthetic nucleotides, such as a peptide nucleic acid (PNA) or locked nucleic acid (LNA). The guide nucleic acid sequence can be a single molecule or a double molecule. In one embodiment, the guide nucleic acid sequence comprises a single guide RNA.

Gene disruption is also possible through base editing technologies. Base editors are capable of making single base pair changes at defined genetic loci to alter gene expression. Adenine base editors (ABEs) and cytosine base editors (CBEs) combine a deaminase enzyme with a Cas nickase to mediate gene editing without double-stranded breaks in DNA. Without wishing to be bound by theory, this method may provide for an efficient targeted multiplexed editing system. In some embodiments, a base editor system comprises a nucleotide binding domain, a deaminase domain for deaminating nucleobases in a target nucleotide sequence; and one or more guide RNA molecules (gRNAs) targeting Cas to a specific locus. Adenine base editors make A to G (or T to C) point mutations at a target site and cytosine base editors make C to T (or G to A) point mutations at a target site. In some cases, cytosine base editors can be fused with an inhibitor of uracil DNA glycosylase (UGI) to prevent base excision repair. In some embodiments, the deaminase is an adenosine deaminase. In some embodiments, the adenosine deaminase catalyzes the hydrolytic deamination of adenine or adenosine in deoxyribonucleic acid (DNA). In some embodiments, the deaminase may be AID, CDA1, or APOBEC3G. In some embodiments, ADE or CBE may create 1, 2, 3, 4, 5, 6, 7, 8, 9, 10, or more simultaneous edits at a genomic target site. In some embodiments, base editors can disrupt gene expression by making point mutations in splicing motifs or start codons. In some embodiments, base editors can disrupt gene expression by making point mutations to create termination codons. In some embodiments, a base editing system described herein may be used to create a CD3 CAR T cell. In some embodiments, the base editor system can comprise a dual base editor (e.g., a fusion of adenine and cytosine base editing components). A dual base editor (e.g., combinatorial base editor or multifunctional base editor) can comprise an adenosine deaminase domain and a cytidine deaminase domain. A dual base editor may further comprise one or more UGIs.

In some embodiments, the engineered immune cell of the present disclosure can be modified via the CRISPR/Cas system to inactivate the human CD3 gene. Details of the genomic structure and sequence of the human CD3 gene can be found, for example, in NCBI Gene database under GeneID No. 916 or UNIPROT ID NO. P07766.

Commercially available kits, gRNA vectors and donor vectors, for knockout of specific target genes are available, for example, from Origene (Rockville, Md.), GenScript (Atlanta, Ga.), Applied Biological Materials (ABM; Richmond, British Colombia), BioCat (Heidelberg, Germany) or others. For example, commercially available kits or kit components for knockout of CD3 via CRISPR include, for example, those available as catalog numbers KN408276, KN410010, and KN420512, each available from OriGene, and those available as catalog numbers sc-401519, sc-400240-KO-2, sc-401519-HDR, sc-419553-NIC, sc-400240-HDR-2, and sc-400240-NIC-2, each available from Santa Cruz Biotechnology.

In some embodiments, the chimeric antigen receptor (CAR) described herein can be introduced into the human CD3 gene locus using the CRISPR/Cas system.

In one aspect, provided is an engineered immune cell comprising: a first nucleic acid that comprises a first nucleotide sequence encoding a CD3 CAR linked to second nucleotide sequence encoding a CD20 protein, e.g., a truncated CD20 protein having an amino acid sequence of SEQ ID NO: 120 or SEQ ID NO: 121 as described herein and a second nucleic acid comprising a third nucleotide sequence encoding a target-binding molecule comprising an antibody or antigen binding fragment that specifically binds CD3 linked to a localizing domain. In some embodiments, the disclosure provides an engineered immune cell comprising: a first nucleic acid that comprises a nucleotide sequence encoding a chimeric antigen receptor (CAR), wherein the CAR comprises intracellular signaling domains of 4-1BB and CD3ζ, and an antibody or antigen binding fragment that specifically binds CD3 and a second nucleic acid comprising a nucleotide sequence encoding a target-binding molecule comprising an antibody or antigen binding fragment that specifically binds CD3 linked to a localizing domain.

In some embodiments, the antibody that binds CD3 in the context of the CAR, as well as in the context of the target-binding molecule comprises: a VH amino acid sequence set forth in SEQ ID NO: 1 and a VL amino acid sequence set forth in SEQ ID NO: 2. As described herein, in certain embodiments, the antibody comprises a VH and a VL having sequence that each comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 1 and 2, respectively. In certain embodiments, the antibody that binds CD3 in the context of the CAR can be different from the antibody that binds CD3 in the context of the target-binding molecule (the protein expression blocker or PEBL), as described herein. In certain embodiments, costimulatory domain of 4-1BB comprises the sequence set forth in SEQ ID NO: 102. In certain embodiments, the cytoplasmic signaling domain of CD3ζ comprises the sequence set forth in SEQ ID NO: 103.

In some embodiments, the antibody is a scFv. In some embodiments, the scFv comprises a VH sequence set forth in SEQ ID NO: 1 and a variable light chain VL sequence set forth in SEQ ID NO: 2. As described herein, in some embodiments, the scFv comprises a VH and a VL having sequence that each comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NO: 1 and 2, respectively. In some embodiments, the CAR further comprises a hinge and transmembrane sequence.

In some embodiments, the CD3 CAR comprises an amino acid sequence of SEQ ID NO: 95-96, 120-121, 104-105, 107-108, 110-111, 125-126, or 128-131. In some embodiments, the CD3 CAR comprises an amino acid sequence having at least one, two or three modifications but not more than 30, 20 or 10 modifications of the amino acid sequence as set forth in any of SEQ ID NOs: 95-96, 120-121, 104-105, 107-108, 110-111, 125-126, or 128-131. In some embodiments, the CD3 CAR comprises an amino acid sequence with 95-99% identity to the amino acid sequence as set forth in any of SEQ ID NOs: 95-96, 120-121, 104-105, 107-108, 110-111, 125-126, or 128-131. In some embodiments, the CD3 CAR comprises a scFv domain comprising an amino acid sequence as set forth in SEQ ID NO: 98, 106, 109, or 112. In some embodiments, the CD3 CAR comprises an amino acid sequence of SEQ ID NO: 1, an amino acid sequence of SEQ ID NO:2, a 4-1BB costimulatory domain (SEQ ID NO: 102), a CD3ζ primary signaling domain (SEQ ID NO: 103), and a CD8 hinge domain (SEQ ID NO: 99) and a CD8 transmembrane domain (SEQ ID NO: 100). In some embodiments, the CD3 CAR also includes a VH-VL linker such as but not limited to a peptide linker having an amino acid sequence as set forth in SEQ ID NO: 85.

In some embodiments, the target binding molecule comprises at least one, at least two, at least three, at least 4, or at least five target binding domains. In some embodiments, the target binding molecule comprises one target binding domain, e.g., a CD3 binding domain. In some embodiments, the target binding molecule comprises a first target binding domain, e.g., a first CD3 binding domain and a second target binding domain, e.g., a second CD3 binding domain. In some embodiments, the first target binding domain and the second target binding domain are identical. In some embodiments, the first target binding domain and the second target binding domain are different. In some embodiments, the first target binding domain and the second target binding domain are connected with a linker, e.g., a peptide linker. In some embodiments, the linker comprises an amino acid sequence as set forth in SEQ ID NO:135. In some embodiments, the intracellular targeting signal of the PEBL comprises one or more of an ER retention sequence, a golgi retention sequence; a proteosome localizing sequence; or a transmembrane domain sequence derived from, 4-1BB, CD28, CD34, CD4, CD8, FcεRIγ, CD16, OX40, CD3ζ, CD3c, CD37, CD36, TCRα, TCRβ, CD32, CD64, VEGFR2, FAS, or FGFR2B.

In some embodiments, the intracellular targeting signal comprises one or more of ER retention peptide, e.g., an ER retention sequence having a sequence of KDEL (SEQ ID NO:137). In some embodiments, the ER retention sequence is fused to a MYC tag (SEQ ID NO: 139) having a sequence as set forth in SEQ ID NO: 141. In some embodiments, the ER retention sequence is linked to a MYC tag having a sequence as set forth in SEQ ID NO: 139. In some embodiments the ER retention sequence is linked to a MYC tag via a linker having a sequence as set forth in SEQ ID NO: 143. In some embodiments, the intracellular targeting signal comprises an ER retention sequence comprising an amino acid sequence as set forth in any one of SEQ ID NOs: 137-143. In some embodiments, the intracellular targeting signal comprises a Golgi retention sequence. In some embodiments, the Golgi retention sequence is any one of SEQ ID NOs: 147-149. In some embodiments, the intracellular targeting signal comprises a proteosome localizing sequence. In some embodiments, the proteosome localizing sequence comprises an amino acid sequence as set forth in SEQ ID NO: 150 or SEQ ID NO: 151. In some embodiments, the PEBL downregulates cell surface expression of endogenous CD3 in the immune cell.

In some embodiments, the CD3 PEBL comprises an amino acid sequence having at least 90% sequence identity or at least 95% sequence identity to SEQ ID NO:152, an amino acid sequence having at least 90% sequence identity or at least 95% sequence identity to SEQ ID NO:153, an amino acid sequence having at least 90% sequence identity or at least 95% sequence identity to SEQ ID NO:154, an amino acid sequence having at least 90% sequence identity or at least 95% sequence identity to SEQ ID NO:155, an amino acid sequence having at least 90% sequence identity or at least 95% sequence identity to SEQ ID NO:156, or an amino acid sequence having at least 90% sequence identity or at least 95% sequence identity to SEQ ID NO:157.

In some embodiments, the TCR PEBL comprises an amino acid sequence having at least 90% sequence identity or at least 95% sequence identity to SEQ ID NO:158, an amino acid sequence having at least 90% sequence identity or at least 95% sequence identity to SEQ ID NO:159.

In some embodiments, the engineered immune cell further comprises a nucleic acid that comprises a nucleotide sequence encoding a target-binding molecule linked to a localizing domain, wherein the target-binding molecule is an antibody or antigen binding fragment that binds CD3, and the localizing domain comprises an ER retention sequence. In some embodiments, the nucleic acid comprises a nucleotide sequence that encodes a target-binding molecule linked to a localizing domain, as described herein. In some embodiments, the target-binding molecule is an antibody that binds CD3. In certain embodiments, the antibody is a scFv. In some embodiments, the scFv comprises a VH sequence having at least 90% sequence identity (e.g., 90%, 91%, 92%, 93%, 94%, 95%, 96%, 97%, 98%, 99%, or more sequence identity) to the sequence of SEQ ID NO: 1 and a VL sequence having at least 90% sequence identity (e.g., 90%, 91%, 92%, 93%, 94%, 95%, 96%, 97%, 98%, 99%, or more sequence identity) to the sequence of SEQ ID NO: 2. In certain embodiments, the scFv comprises a VH sequence set forth in SEQ ID NO: 1 and a VL sequence set forth in SEQ ID NO: 2.

In some embodiments, the target binding molecule comprises at least one, at least two, at least three, at least four, or at least five target binding domains. In some embodiments, the target binding molecule comprises one target binding domain, e.g., a CD3 binding domain. In some embodiments, the target binding molecule comprises a first target binding domain, e.g., a first CD3 binding domain and a second target binding domain, e.g., a second CD3 binding domain. In some embodiments, the first target binding domain and the second target binding domain are identical. In some embodiments, the first target binding domain and the second target binding domain are different. In some embodiments, the first target binding domain and the second target binding domain are connected with a linker, e.g., a peptide linker. In some embodiments, the linker comprises an amino acid sequence as set forth in SEQ ID NO: 135. In some embodiments, the localizing domain further comprises one or more of myc tag (SEQ ID NO: 139), ER retention peptide KDEL (SEQ ID NO:137), localization domain KDEL (SEQ ID NO: 137) tethered to scFv with myc (“myc KDEL”) (SEQ ID NO: 141), or ER retention sequence (SEQ ID NO: 143).

In some embodiments, the engineered immune cell co-expresses the CD3 CAR and the CD3 PEBL. In some embodiments, the engineered immune cell expresses the CD3 CAR followed by the CD3 PEBL expression. In some embodiments, the engineered immune cell expresses the CD3 PEBL followed by the CD3 CAR expression.

According to a further aspect, engineered immune cells are provided wherein fratricide is reduced and/or prevented. These cells are characterized by functional inhibition of TCR/CD3 signaling, e.g., CD3 retained within the cells, e.g., through knockout of CD3 or by permanent or transient inhibition of CD3 expression. In some embodiments, the engineered immune cells are resistant to self-killing. In some embodiments, reduction can be expressed as a percentage reduction compared to control, e.g., there is 10%, 20%, 25%, 30%, 40%, 50%, 60%, 70%, 75%, 80%, 90% or even 100% less fratricide. In some embodiments, reduction of fratricide can be evaluated by increase in final cell yield or number e.g., by an increase in cell yield of 10%, 20%, 25%, 30%, 40%, 50%, 60%, 70%, 75%, 80%, 90%, 100% or even more than 100%. In some embodiments, reduction of fratricide as defined herein can also be evaluated by increase in antigen-specific cytokine production (e.g., interferon gamma secretion) and/or, in case of T cells, an increased frequency in naive phenotype of T cells (CD62L+/CD45RA+). In some embodiments, engineered immune cells described herein may show an increase in secretion of one or more cytokines in the presence of CD3+ target cells. The one or more cytokines can be IFN-γ, IL-2, TNF-α, IL-10, IL-17, CD107a, CCL3, CCL4, IL-5, IL-13, IL-9, IL-17A, IL-17F, IL-4, IL-22, or a combination thereof. In some embodiments, engineered immune cells described herein may show an increase in secretion of IFN-γ, IL-2, and/or TNF-α in the presence of CD3+ target cells compared to identical cells that do not express the CD3 CAR, CD3 PEBL, and/or kill gene. Methods for quantification and analysis of cytokine secretion may include fluorescent activated cell sorting (FACS), flow cytometry, immunohistochemistry, Luminex, Olink, ELISA, and/or ELISPOT assay. In some embodiments, reduction of fratricide can be evaluated by an increase in antigen-specific cytokine production of 10%, 20%, 25%, 30%, 40%, 50%, 60%, 70%, 75%, 80%, 90%, 100% or even more than 100%. In some embodiments, reduction of fratricide can be evaluated by e.g., an increased frequency of naive T cells of 10%, 20%, 25%, 30%, 40%, 50%, 60%, 70%, 75%, 80%, 90%, 100% or even more than 100%. Measures to reduce fratricide can be taken prior to the start of the fratricide process or during the fratricide killing. Preventing fratricide refers to measures taken before the fratricide process starts. According to particular embodiments, absolute prevention of fratricide means that no CD3-induced fratricide takes place equivalent to a 100% reduction in fratricide.

A T cell may comprise a CAR molecule without a PEBL molecule. A T cell may comprise an anti-CD3 CAR molecule without a CD3 PEBL. In some embodiments, engineered immune cells expressing anti-CD3 CAR without expressing CD3 PEBL may lead to fratricide in population of engineered immune cells. Engineered immune cells expressing anti-CD3 CAR without expressing CD3 PEBL may show a 5-fold, a 10-fold, a 15-fold, a 20-fold, a 25-fold, a 30-fold, a 40-fold, or a 50-fold reduction in cell number compared to that of non-transduced controls or engineered immune cells expressing anti-CD3 CAR and CD3 PEBL.

Codon usage bias has been reported for numerous organisms, from viruses to eukaryotes. Since the genetic code is degenerate (i.e., each amino acid can be coded by on average three different codons), the DNA sequence can be modified by synonymous nucleotide substitutions without altering the amino acid sequence of the encoded protein. Such synonymous codon optimization has been performed for the purpose of optimizing expression in a desired host, as described in the scientific literature and in patent documents. See, U.S. Pat. Nos. 5,786,464 and 6,114,14. In some embodiments, the nucleic acid described herein may be modified to improve cloning efficiency. In some embodiments, the nucleic acids described herein are subjected to codon optimization to increase the efficiency of gene expression. In some embodiments, the CD3 binding domain of the CD3 CAR and/or CD3 PEBL are encoded by a nucleic acid whose sequence has been codon optimized for expression in a mammalian cell. In some embodiments, the CD3 CARs described herein are encoded by nucleic acids that have been codon optimized for expression in a mammalian cell. In some embodiments, the CD3 PEBLs described herein are encoded by nucleic acids that have been codon optimized for expression in a mammalian cell.

In some embodiments, the disclosure provides a method to downregulate TCR and CD3 expression on the surface of an immune cell by a target binding molecule, e.g., a PEBL, e.g., a CD3 PEBL or TCR PEBL. In some embodiments, the target binding molecule, e.g., CD3 PEBL downregulates CD38 expression. In some embodiments, the target binding molecule, e.g., CD3 PEBL downregulates CD3 and/or TCR expression on the surface of an immune cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 99.9% or by 100%. In some embodiments, the target binding molecule downregulates CD3 and/or TCR expression for at least 6 hours, at least 12 hours, at least 18 hours, at least 24 hours, at least 1 day, at least 2 days, at least 3 days, at least 4 days, at least 5 days, at least 10 days, at least 15 days, at least 20 days, at least 25 days, at least 30 days, or indefinitely.

In some embodiments, the disclosure provides a method to reduce fratricide by expressing a CD3 PEBL in combination with a CD3 CAR in an immune cell, e.g., a T cell. In some embodiments, the CD3 PEBL and the CD3 CAR are expressed together. In some embodiments, the CD3 PEBL and the CD3 CAR are expressed sequentially.

In other aspects, also provided is a method of treating a disease in a subject in need thereof, comprising administering a therapeutic amount of an engineered immune cell having any of the embodiments described herein to the subject, thereby treating the disease in a subject in need thereof. In some embodiments, the disease is a cancer. In some embodiments, the disease is an immune disease, e.g., an autoimmune disease.

In certain embodiments, the method comprises administering a therapeutic amount of an engineered immune cell comprising a nucleic acid that comprises a nucleotide sequence encoding a CAR, e.g., CD3 CAR, as described herein.

In certain embodiments, the method comprises administering a therapeutic amount of an engineered immune cell that further comprises a nucleic acid having a nucleotide sequence encoding PEBL, as described herein (e.g., CD3 PEBL or TCR PEBL).

In some embodiments, the method of treating a disease in a subject in need thereof comprises administering a therapeutic composition comprising a population of immune cells, e.g., engineered immune cells comprising a first nucleotide sequence encoding a CAR, e.g., CD3 CAR, as described herein and a second nucleic acid having a nucleotide sequence encoding PEBL, as described herein, e.g., a CD3 PEBL to a subject in need thereof thereby treating the subject. In some embodiments, disease in the subject is reduced compared to an identical subject treated with a therapeutic composition comprising a cell population comprising immune cells expressing the CAR and not expressing the PEBL. In some embodiments, a depleting (triggering) agent, e.g., an anti-CD20 antibody (e.g., rituximab) is further administered to activate a kill gene (e.g., CD20 or a truncated fragment thereof). The kill gene may be linked to the CAR, e.g., CD3 CAR. In some embodiments, addition of the triggering agent leads to cytolysis of the engineered immune cells, thereby allowing endogenous T cells of the subject to grow. The anti-CD20 antibody may comprise ofatumumab, rituximab, tositumomab, or obinutuzumab.

In some embodiments, triggering of the kill gene with an anti-CD20 antibody (e.g., rituximab) may deplete a cell population as described herein. In some embodiments, administration of an anti-CD20 antibody (e.g., rituximab) may deplete a cell population as described herein by 5%, 10%, 20%, 25%, 30%, 40%, 50%, 60%, 70%, 80%, 90%, 95%, 99%, or 100%. An anti-CD20 antibody (e.g., rituximab) may be administered in one dose, two doses, three doses, four doses, five doses, six doses, or more. Rituximab may be administered at a dosage of at least about 0.1 mg/kg, at least about 0.5 mg/kg, at least about 1 mg/kg, at least about 2.5 mg/kg, at least about 5 mg/kg, at least about 10 mg/kg, at least about 15 mg/kg, at least about 20 mg/kg, at least about 25 mg/kg, at least about 30 mg/kg, at least about 40 mg/kg, or greater than about 40 mg/kg. Rituximab may be administered at a dosage of at most about 40 mg/kg, at most about 30 mg/kg, at most about 25 mg/kg, at most about 20 mg/kg, at most about 15 mg/kg, at most about 10 mg/kg, at most about 5 mg/kg, at most about 2.5 mg/kg, at most about 1 mg/kg, at most about 0.5 mg/kg, at most about 0.1 mg/kg, or less than about 0.1 mg/kg. Rituximab may be administered at a dosage from about 0.01 mg/kg to about 40 mg/kg. Rituximab may be administered at a dosage from about 0.01 mg/kg to about 0.1 mg/kg, about 0.01 mg/kg to about 0.5 mg/kg, about 0.01 mg/kg to about 1 mg/kg, about 0.01 mg/kg to about 2.5 mg/kg, about 0.01 mg/kg to about 5 mg/kg, about 0.01 mg/kg to about 10 mg/kg, about 0.01 mg/kg to about 15 mg/kg, about 0.01 mg/kg to about 20 mg/kg, about 0.01 mg/kg to about 25 mg/kg, about 0.01 mg/kg to about 30 mg/kg, about 0.01 mg/kg to about 40 mg/kg, about 0.1 mg/kg to about 0.5 mg/kg, about 0.1 mg/kg to about 1 mg/kg, about 0.1 mg/kg to about 2.5 mg/kg, about 0.1 mg/kg to about 5 mg/kg, about 0.1 mg/kg to about 10 mg/kg, about 0.1 mg/kg to about 15 mg/kg, about 0.1 mg/kg to about 20 mg/kg, about 0.1 mg/kg to about 25 mg/kg, about 0.1 mg/kg to about 30 mg/kg, about 0.1 mg/kg to about 40 mg/kg, about 0.5 mg/kg to about 1 mg/kg, about 0.5 mg/kg to about 2.5 mg/kg, about 0.5 mg/kg to about 5 mg/kg, about 0.5 mg/kg to about 10 mg/kg, about 0.5 mg/kg to about 15 mg/kg, about 0.5 mg/kg to about 20 mg/kg, about 0.5 mg/kg to about 25 mg/kg, about 0.5 mg/kg to about 30 mg/kg, about 0.5 mg/kg to about 40 mg/kg, about 1 mg/kg to about 2.5 mg/kg, about 1 mg/kg to about 5 mg/kg, about 1 mg/kg to about 10 mg/kg, about 1 mg/kg to about 15 mg/kg, about 1 mg/kg to about 20 mg/kg, about 1 mg/kg to about 25 mg/kg, about 1 mg/kg to about 30 mg/kg, about 1 mg/kg to about 40 mg/kg, about 2.5 mg/kg to about 5 mg/kg, about 2.5 mg/kg to about 10 mg/kg, about 2.5 mg/kg to about 15 mg/kg, about 2.5 mg/kg to about 20 mg/kg, about 2.5 mg/kg to about 25 mg/kg, about 2.5 mg/kg to about 30 mg/kg, about 2.5 mg/kg to about 40 mg/kg, about 5 mg/kg to about 10 mg/kg, about 5 mg/kg to about 15 mg/kg, about 5 mg/kg to about 20 mg/kg, about 5 mg/kg to about 25 mg/kg, about 5 mg/kg to about 30 mg/kg, about 5 mg/kg to about 40 mg/kg, about 10 mg/kg to about 15 mg/kg, about 10 mg/kg to about 20 mg/kg, about 10 mg/kg to about 25 mg/kg, about 10 mg/kg to about 30 mg/kg, about 10 mg/kg to about 40 mg/kg, about 15 mg/kg to about 20 mg/kg, about 15 mg/kg to about 25 mg/kg, about 15 mg/kg to about 30 mg/kg, about 15 mg/kg to about 40 mg/kg, about 20 mg/kg to about 25 mg/kg, about 20 mg/kg to about 30 mg/kg, about 20 mg/kg to about 40 mg/kg, about 25 mg/kg to about 30 mg/kg, about 25 mg/kg to about 40 mg/kg, or about 30 mg/kg to about 40 mg/kg.

Rituximab may be administered intravenously to a subject in need thereof. Rituximab may be administered at a dosage of at least about 0.1 mg/mL, at least about 0.5 mg/mL, at least about 1 mg/mL, at least about 2.5 mg/mL, at least about 5 mg/mL, at least about 10 mg/mL, at least about 15 mg/mL, at least about 20 mg/mL, at least about 25 mg/mL, at least about 30 mg/mL, at least about 40 mg/mL, or greater than about 40 mg/mL. Rituximab may be administered at a dosage of at most about 40 mg/mL, at most about 30 mg/mL, at most about 25 mg/mL, at most about 20 mg/mL, at most about 15 mg/mL, at most about 10 mg/mL, at most about 5 mg/mL, at most about 2.5 mg/mL, at most about 1 mg/mL, at most about 0.5 mg/mL, at most about 0.1 mg/mL, or less than about 0.1 mg/mL. Rituximab may be administered at a dosage from about 0.01 mg/mL to about 40 mg/mL. Rituximab may be administered at a dosage from about 0.01 mg/mL to about 0.1 mg/mL, about 0.01 mg/mL to about 0.5 mg/mL, about 0.01 mg/mL to about 1 mg/mL, about 0.01 mg/mL to about 2.5 mg/mL, about 0.01 mg/mL to about 5 mg/mL, about 0.01 mg/mL to about 10 mg/mL, about 0.01 mg/mL to about 15 mg/mL, about 0.01 mg/mL to about 20 mg/mL, about 0.01 mg/mL to about 25 mg/mL, about 0.01 mg/mL to about 30 mg/mL, about 0.01 mg/mL to about 40 mg/mL, about 0.1 mg/mL to about 0.5 mg/mL, about 0.1 mg/mL to about 1 mg/mL, about 0.1 mg/mL to about 2.5 mg/mL, about 0.1 mg/mL to about 5 mg/mL, about 0.1 mg/mL to about 10 mg/mL, about 0.1 mg/mL to about 15 mg/mL, about 0.1 mg/mL to about 20 mg/mL, about 0.1 mg/mL to about 25 mg/mL, about 0.1 mg/mL to about 30 mg/mL, about 0.1 mg/mL to about 40 mg/mL, about 0.5 mg/mL to about 1 mg/mL, about 0.5 mg/mL to about 2.5 mg/mL, about 0.5 mg/mL to about 5 mg/mL, about 0.5 mg/mL to about 10 mg/mL, about 0.5 mg/mL to about 15 mg/mL, about 0.5 mg/mL to about 20 mg/mL, about 0.5 mg/mL to about 25 mg/mL, about 0.5 mg/mL to about 30 mg/mL, about 0.5 mg/mL to about 40 mg/mL, about 1 mg/mL to about 2.5 mg/mL, about 1 mg/mL to about 5 mg/mL, about 1 mg/mL to about 10 mg/mL, about 1 mg/mL to about 15 mg/mL, about 1 mg/mL to about 20 mg/mL, about 1 mg/mL to about 25 mg/mL, about 1 mg/mL to about 30 mg/mL, about 1 mg/mL to about 40 mg/mL, about 2.5 mg/mL to about 5 mg/mL, about 2.5 mg/mL to about 10 mg/mL, about 2.5 mg/mL to about 15 mg/mL, about 2.5 mg/mL to about 20 mg/mL, about 2.5 mg/mL to about 25 mg/mL, about 2.5 mg/mL to about 30 mg/mL, about 2.5 mg/mL to about 40 mg/mL, about 5 mg/mL to about 10 mg/mL, about 5 mg/mL to about 15 mg/mL, about 5 mg/mL to about 20 mg/mL, about 5 mg/mL to about 25 mg/mL, about 5 mg/mL to about 30 mg/mL, about 5 mg/mL to about 40 mg/mL, about 10 mg/mL to about 15 mg/mL, about 10 mg/mL to about 20 mg/mL, about 10 mg/mL to about 25 mg/mL, about 10 mg/mL to about 30 mg/mL, about 10 mg/mL to about 40 mg/mL, about 15 mg/mL to about 20 mg/mL, about 15 mg/mL to about 25 mg/mL, about 15 mg/mL to about 30 mg/mL, about 15 mg/mL to about 40 mg/mL, about 20 mg/mL to about 25 mg/mL, about 20 mg/mL to about 30 mg/mL, about 20 mg/mL to about 40 mg/mL, about 25 mg/mL to about 30 mg/mL, about 25 mg/mL to about 40 mg/mL, or about 30 mg/mL to about 40 mg/mL.

In some embodiments, a therapeutic composition comprising the cell population disclosed herein may comprise a pharmaceutically acceptable excipient. A pharmaceutically acceptable excipient can comprise any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

The therapeutic composition may be administered to a subject in need thereof. A therapeutic composition described herein may be administered via routes including, but not limited to, subcutaneous, intradermal, intralesional, intraarticular, intraperitoneal, intravesical, transmucosal, intraorgan, intrathecal, intramuscular, intravenous, and intravascular. In some embodiments, the therapeutic composition may be administered locally to a diseased site (e.g., tumor site). A therapeutic composition may be administered as one dose. A therapeutic composition may be administered as multiple doses. A therapeutic composition may be administered as at least about one dose, at least about two doses, at least about three doses, at least about four doses, at least about five doses, at least about six doses, at least about seven doses, at least about eight doses, at least about nine doses, or at least about ten doses.

In certain embodiments, the cancer is a T cell malignancy, e.g., T cell leukemia or T cell lymphoma, such as T-cell acute lymphoblastic leukemia, T-cell prolymphocytic leukemia, T-cell large granular lymphocytic leukemia, enteropathy-associated T-cell lymphoma, hepatosplenic T-cell lymphoma, subcutaneous panniculitis-like T-cell lymphoma, mycosis fungoides, Sezary syndrome, primary cutaneous gamma-delta T-cell lymphoma, peripheral T-cell lymphoma not otherwise specified, angioimmunoblastic T-cell lymphoma, anaplastic large cell lymphoma. In one embodiment, the cancer is a Peripheral T-Cell Lymphoma (PTCL).

In some embodiments, the engineered immune cell is autologous to the subject in need of treatment, e.g., cancer treatment. In other embodiments, the engineered immune cell is allogeneic to the subject in need of treatment.

In certain embodiments, the method of treating cancer according to the present disclosure is combined with at least one other known cancer therapy, e.g., radiotherapy, chemotherapy, or other immunotherapy.

In other aspects, also provided is use of an engineered immune cell of any of the embodiments described herein for treating cancer, comprising administering an effective amount of the engineered immune cell to a subject in need thereof. In certain embodiments, the cancer is a T cell malignancy. In certain embodiments, the T cell malignancy is early T-cell progenitor acute lymphoblastic leukemia (ETP-ALL) or peripheral T cell lymphoma (PTCL).

In some embodiments, engineered immune cell, cell population, or therapeutic composition as described herein may be used to reduce the risk or reduce the symptoms of graft versus host disease in a subject. In some embodiments, engineered immune cell, cell population, or therapeutic composition as described herein may be used to minimize the risk or minimize the symptoms of graft versus host disease in a subject. In some embodiments, graft versus host disease (e.g., symptoms of graft versus host disease) may be reduced by at least about 1%, at least about 5%, at least about 10%, at least about 15%, at least about 20%, at least about 30%, at least about 40%, at least about 50%, at least about 60%, at least about 70%, at least about 80%, at least about 90%, or at least about 100% in a subject administered the therapeutic composition (e.g., cell population and/or engineered immune cell) described herein compared to graft versus host disease (e.g., symptoms of graft versus host disease) in an identical subject treated with a therapeutic composition comprising a cell population comprising immune cells expressing the CAR and not expressing the PEBL as described herein. In some embodiments, graft versus host disease (e.g., symptoms of graft versus host disease) may be reduced by at most about 100%, at most about 90%, at most about 80%, at most about 70%, at most about 60%, at most about 50%, at most about 40%, at most about 30%, at most about 20%, at most about 15%, at most about 10%, at most about 5%, or at most about 1% in a subject administered the therapeutic composition (e.g., cell population and/or engineered immune cell) described herein compared to graft versus host disease (e.g., symptoms of graft versus host disease) in an identical subject treated with a therapeutic composition comprising a cell population comprising immune cells expressing the CAR and not expressing the PEBL as described herein.

In some embodiments, weight loss may be used as a metric for measuring graft versus host disease (e.g., xenoreactivity or alloreactivity) in a subject. Administration of the therapeutic composition (e.g., cell population and/or engineered immune cell) described herein to a subject in need thereof may reduce a subject's weight by less than about 1%, less than about 2%, less than about 3%, less than about 4%, less than about 5%, less than about 10%, less than about 15%, less than about 20%, less than about 25%, or less than about 30% compared to the weight change of an identical subject treated with a therapeutic composition comprising a cell population comprising immune cells expressing the CAR and not expressing the PEBL as described herein. In some embodiments, administration of the therapeutic composition (e.g., cell population and/or engineered immune cell) described herein to a subject in need thereof may reduce a subject's weight by less than about 1%, less than about 2%, less than about 3%, less than about 4%, less than about 5%, less than about 10%, less than about 15%, less than about 20%, less than about 25%, or less than about 30% compared to the weight change of an identical subject receiving no treatment.

A reduction in platelet count (e.g., thrombocytopenia) may be a symptom of GvHD. In some embodiments, platelet count may be used as a metric for measuring graft versus host disease (e.g., xenoreactivity or alloreactivity) in a subject. A platelet count of a subject administered the therapeutic composition (e.g., cell population and/or engineered immune cell) described herein may be at least about 0.01 million counts/microliter (μl), at least about 0.025 million counts/μl, at least about 0.05 million counts/μl, at least about 0.075 million counts/μl, at least about 0.1 million counts/μl, at least about 0.2 million counts/μl, at least about 0.3 million counts/μl, at least about 0.4 million counts/μl, at least about 0.5 million counts/μl, at least about 0.6 million counts/μl, at least about 0.7 million counts/μl, at least about 0.8 million counts/μl, at least about 0.9 million counts/μl, at least about 1.0 million counts/μl, at least about 1.2 million counts/μl, at least about 1.5 million counts/μl, at least about 1.8 million counts/μl, at least about 2.0 million counts/μl, or greater than about 2.0 million counts/μl, compared to the platelet count of an identical subject treated with a therapeutic composition comprising a cell population comprising immune cells expressing the CAR and not expressing the PEBL as described herein. A platelet count of a subject administered the therapeutic composition (e.g., cell population and/or engineered immune cell) described herein may be at most about 2.0 million counts/μl, at most about 1.8 million counts/μl, at most about 1.5 million counts/μl, at most about 1.2 million counts/μl, at most about 1.0 million counts/μl, at most about 0.9 million counts/μl, at most about 0.8 million counts/μl, at most about 0.7 million counts/μl, at most about 0.6 million counts/μl, at most about 0.5 million counts/μl, at most about 0.4 million counts/μl, at most about 0.3 million counts/μl, at most about 0.2 million counts/μl, at most about 0.1 million counts/μl, at most about 0.075 million counts/μl, at most about 0.05 million counts/μl, at most about 0.025 million counts/μl, at most about 0.01 million counts/μl, or less than about 0.01 million counts/μl, compared to the platelet count of an identical subject treated with a therapeutic composition comprising a cell population comprising immune cells expressing the CAR and not expressing the PEBL as described herein. A platelet count of a subject administered the therapeutic composition (e.g., cell population and/or engineered immune cell) described herein may be at least about 0.01 million counts/μl, at least about 0.025 million counts/μl, at least about 0.05 million counts/μl, at least about 0.075 million counts/μl, at least about 0.1 million counts/μl, at least about 0.2 million counts/μl, at least about 0.3 million counts/μl, at least about 0.4 million counts/μl, at least about 0.5 million counts/μl, at least about 0.6 million counts/μl, at least about 0.7 million counts/μl, at least about 0.8 million counts/μl, at least about 0.9 million counts/μl, at least about 1.0 million counts/μl, at least about 1.2 million counts/μl, at least about 1.5 million counts/μl, at least about 1.8 million counts/μl, or at least about 2.0 million counts/μl compared to the platlet count of an identical subject receiving no treatment. A platelet count of a subject administered the therapeutic composition (e.g., cell population and/or engineered immune cell) described herein may be at most about 2.0 million counts/μl, at most about 1.8 million counts/μl, at most about 1.5 million counts/μl, at most about 1.2 million counts/μl, at most about 1.0 million counts/μl, at most about 0.9 million counts/μl, at most about 0.8 million counts/μl, at most about 0.7 million counts/μl, at most about 0.6 million counts/μl, at most about 0.5 million counts/μl, at most about 0.4 million counts/μl, at most about 0.3 million counts/μl, at most about 0.2 million counts/μl, at most about 0.1 million counts/μl, at most about 0.075 million counts/μl, at most about 0.05 million counts/μl, at most about 0.025 million counts/μl, at most about 0.01 million counts/μl, or less than about 0.01 million counts/μl, compared to the platlet count of an identical subject receiving no treatment.

A reduction in hemoglobin level may be a symptom of GvHD. In some embodiments, hemoglobin levels may be used as a metric for measuring graft versus host disease (e.g., xenoreactivity or alloreactivity) in a subject. Hemoglobin level of a subject administered the therapeutic composition (e.g., cell population and/or engineered immune cell) described herein may be at least about 250 mg/dL, at least about 500 mg/dL, at least about 750 mg/dL, at least about 1000 mg/dL, at least about 2000 mg/dL, at least about 3000 mg/dL, at least about 4000 mg/dL, at least about 5000 mg/dL, at least about 7500 mg/dL, at least about 10,000 mg/dL, at least about 11,000 mg/dL, at least about 12,000 mg/dL, at least about 13,000 mg/dL, at least about 14,000 mg/dL, at least about 15,000 mg/dL, at least about 16,000 mg/dL, at least about 17,000 mg/dL, at least about 18,000 mg/dL, at least about 19,000 mg/dL, at least about 20,000 mg/dL, or greater than about 20,000 mg/dL. Hemoglobin level of a subject administered the therapeutic composition (e.g., cell population and/or engineered immune cell) described herein may be at most about 20,000 mg/dL, at most about 19,000 mg/dL, at most about 18,000 mg/dL, at most about 17,000 mg/dL, at most about 16,000 mg/dL, at most about 15,000 mg/dL, at most about 14,000 mg/dL, at most about 13,000 mg/dL, at most about 12,000 mg/dL, at most about 11,000 mg/dL, at most about 10,000 mg/dL, at most about 7500 mg/dL, at most about 5000 mg/dL, at most about 4000 mg/dL, at most about 3000 mg/dL, at most about 2000 mg/dL, at most about 1000 mg/dL, at most about 750 mg/dL, at most about 500 mg/dL, at most about 250 mg/dL, or less than about 250 mg/dL. Hemoglobin level of a subject administered the therapeutic composition (e.g., cell population and/or engineered immune cell) described herein may be from about 250 mg/dL to about 10,000 mg/dL. Hemoglobin level of a subject administered the therapeutic composition (e.g., cell population and/or engineered immune cell) described herein may be from about 250 mg/dL to about 500 mg/dL, about 250 mg/dL to about 750 mg/dL, about 250 mg/dL to about 1,000 mg/dL, about 250 mg/dL to about 1,500 mg/dL, about 250 mg/dL to about 2,500 mg/dL, about 250 mg/dL to about 5,000 mg/dL, about 250 mg/dL to about 6,000 mg/dL, about 250 mg/dL to about 7,000 mg/dL, about 250 mg/dL to about 8,000 mg/dL, about 250 mg/dL to about 9,000 mg/dL, about 250 mg/dL to about 10,000 mg/dL, about 500 mg/dL to about 750 mg/dL, about 500 mg/dL to about 1,000 mg/dL, about 500 mg/dL to about 1,500 mg/dL, about 500 mg/dL to about 2,500 mg/dL, about 500 mg/dL to about 5,000 mg/dL, about 500 mg/dL to about 6,000 mg/dL, about 500 mg/dL to about 7,000 mg/dL, about 500 mg/dL to about 8,000 mg/dL, about 500 mg/dL to about 9,000 mg/dL, about 500 mg/dL to about 10,000 mg/dL, about 750 mg/dL to about 1,000 mg/dL, about 750 mg/dL to about 1,500 mg/dL, about 750 mg/dL to about 2,500 mg/dL, about 750 mg/dL to about 5,000 mg/dL, about 750 mg/dL to about 6,000 mg/dL, about 750 mg/dL to about 7,000 mg/dL, about 750 mg/dL to about 8,000 mg/dL, about 750 mg/dL to about 9,000 mg/dL, about 750 mg/dL to about 10,000 mg/dL, about 1,000 mg/dL to about 1,500 mg/dL, about 1,000 mg/dL to about 2,500 mg/dL, about 1,000 mg/dL to about 5,000 mg/dL, about 1,000 mg/dL to about 6,000 mg/dL, about 1,000 mg/dL to about 7,000 mg/dL, about 1,000 mg/dL to about 8,000 mg/dL, about 1,000 mg/dL to about 9,000 mg/dL, about 1,000 mg/dL to about 10,000 mg/dL, about 1,500 mg/dL to about 2,500 mg/dL, about 1,500 mg/dL to about 5,000 mg/dL, about 1,500 mg/dL to about 6,000 mg/dL, about 1,500 mg/dL to about 7,000 mg/dL, about 1,500 mg/dL to about 8,000 mg/dL, about 1,500 mg/dL to about 9,000 mg/dL, about 1,500 mg/dL to about 10,000 mg/dL, about 2,500 mg/dL to about 5,000 mg/dL, about 2,500 mg/dL to about 6,000 mg/dL, about 2,500 mg/dL to about 7,000 mg/dL, about 2,500 mg/dL to about 8,000 mg/dL, about 2,500 mg/dL to about 9,000 mg/dL, about 2,500 mg/dL to about 10,000 mg/dL, about 5,000 mg/dL to about 6,000 mg/dL, about 5,000 mg/dL to about 7,000 mg/dL, about 5,000 mg/dL to about 8,000 mg/dL, about 5,000 mg/dL to about 9,000 mg/dL, about 5,000 mg/dL to about 10,000 mg/dL, about 6,000 mg/dL to about 7,000 mg/dL, about 6,000 mg/dL to about 8,000 mg/dL, about 6,000 mg/dL to about 9,000 mg/dL, about 6,000 mg/dL to about 10,000 mg/dL, about 7,000 mg/dL to about 8,000 mg/dL, about 7,000 mg/dL to about 9,000 mg/dL, about 7,000 mg/dL to about 10,000 mg/dL, about 8,000 mg/dL to about 9,000 mg/dL, about 8,000 mg/dL to about 10,000 mg/dL, or about 9,000 mg/dL to about 10,000 mg/dL.

Hemoglobin level of a subject administered the therapeutic composition (e.g., cell population and/or engineered immune cell) described herein may be from about 10,000 mg/dL to about 20,000 mg/dL. Hemoglobin level of a subject administered the therapeutic composition (e.g., cell population and/or engineered immune cell) described herein may be from about 10,000 mg/dL to about 11,000 mg/dL, about 10,000 mg/dL to about 12,000 mg/dL, about 10,000 mg/dL to about 13,000 mg/dL, about 10,000 mg/dL to about 14,000 mg/dL, about 10,000 mg/dL to about 15,000 mg/dL, about 10,000 mg/dL to about 16,000 mg/dL, about 10,000 mg/dL to about 17,000 mg/dL, about 10,000 mg/dL to about 18,000 mg/dL, about 10,000 mg/dL to about 19,000 mg/dL, about 10,000 mg/dL to about 20,000 mg/dL, about 11,000 mg/dL to about 12,000 mg/dL, about 11,000 mg/dL to about 13,000 mg/dL, about 11,000 mg/dL to about 14,000 mg/dL, about 11,000 mg/dL to about 15,000 mg/dL, about 11,000 mg/dL to about 16,000 mg/dL, about 11,000 mg/dL to about 17,000 mg/dL, about 11,000 mg/dL to about 18,000 mg/dL, about 11,000 mg/dL to about 19,000 mg/dL, about 11,000 mg/dL to about 20,000 mg/dL, about 12,000 mg/dL to about 13,000 mg/dL, about 12,000 mg/dL to about 14,000 mg/dL, about 12,000 mg/dL to about 15,000 mg/dL, about 12,000 mg/dL to about 16,000 mg/dL, about 12,000 mg/dL to about 17,000 mg/dL, about 12,000 mg/dL to about 18,000 mg/dL, about 12,000 mg/dL to about 19,000 mg/dL, about 12,000 mg/dL to about 20,000 mg/dL, about 13,000 mg/dL to about 14,000 mg/dL, about 13,000 mg/dL to about 15,000 mg/dL, about 13,000 mg/dL to about 16,000 mg/dL, about 13,000 mg/dL to about 17,000 mg/dL, about 13,000 mg/dL to about 18,000 mg/dL, about 13,000 mg/dL to about 19,000 mg/dL, about 13,000 mg/dL to about 20,000 mg/dL, about 14,000 mg/dL to about 15,000 mg/dL, about 14,000 mg/dL to about 16,000 mg/dL, about 14,000 mg/dL to about 17,000 mg/dL, about 14,000 mg/dL to about 18,000 mg/dL, about 14,000 mg/dL to about 19,000 mg/dL, about 14,000 mg/dL to about 20,000 mg/dL, about 15,000 mg/dL to about 16,000 mg/dL, about 15,000 mg/dL to about 17,000 mg/dL, about 15,000 mg/dL to about 18,000 mg/dL, about 15,000 mg/dL to about 19,000 mg/dL, about 15,000 mg/dL to about 20,000 mg/dL, about 16,000 mg/dL to about 17,000 mg/dL, about 16,000 mg/dL to about 18,000 mg/dL, about 16,000 mg/dL to about 19,000 mg/dL, about 16,000 mg/dL to about 20,000 mg/dL, about 17,000 mg/dL to about 18,000 mg/dL, about 17,000 mg/dL to about 19,000 mg/dL, about 17,000 mg/dL to about 20,000 mg/dL, about 18,000 mg/dL to about 19,000 mg/dL, about 18,000 mg/dL to about 20,000 mg/dL, or about 19,000 mg/dL to about 20,000 mg/dL.

In another aspect, also provided is a method for producing the engineered immune cell having any of the embodiments described herein, the method comprising introducing into an immune cell, a first nucleic acid that comprises a nucleotide sequence encoding a CAR, e.g., a CD3 CAR.

In certain embodiments, the method further comprises introducing into the immune cell a second nucleic acid that comprises a nucleotide sequence encoding a target-binding molecule linked to a localizing domain (e.g., TCR PEBL or CD3 PEBL). In certain embodiments, the nucleotide sequence encoding CAR and the nucleotide sequence encoding the target-binding molecule are introduced on a single plasmid.

In one aspect, the disclosure provides a method of manufacturing a cellular composition comprising: obtaining a cell population comprising immune cells, introducing a first polynucleotide into the immune cells and a introducing a second polynucleotide into the immune cells wherein the first polynucleotide encodes a PEBL, and the second polynucleotide encodes a CAR. In some embodiments, the first nucleotide and second nucleotide are introduced to the immune cell simultaneously. In some embodiments, the first and second nucleic acid are introduced to the immune cell sequentially. In some embodiments, the polynucleotide encoding the PEBL, e.g., CD3 PEBL is introduced to the immune cell before introducing the polynucleotide encoding the CAR, e.g., CD3 CAR to the immune cell. In some embodiments the polynucleotide encoding the PEBL, e.g., CD3 PEBL is introduced to the immune cell about 2 days (e.g., 48 hours, 46-50 hours, 44-52 hours, or 42-54 hours) before introducing the polynucleotide encoding the CAR, e.g., CD3 CAR to the immune cell. In some embodiments, the cellular composition comprises the engineered immune cells described herein.

In some embodiments, the CD3 PEBL and the CD3 CAR are expressed by transduction of viral vectors (e.g., retroviral vectors, lentiviral vectors) encoding the CD3 PEBL and the CD3 CAR. In some embodiments, the vector encoding the CD3 PEBL is transduced about two days before the vector encoding the CD3 CAR. In some embodiments, the vector encoding the CD3 PEBL is transduced 48 hours, 47-49 hours, 46-50 hours, 45-51 hours, 44-52 hours, or 42-54 hours before the vector encoding the CD3 CAR. In some embodiments, the CD3 PEBL is transduced at least 30, 32, 34, 36, 38, 40, 42, 44, 46, or 48 hours before the CD3 CAR. In some embodiments, the CD3 PEBL is transduced no more than 49, 50, 52, 54, 56, 58, 60, 66, 72, or 78 hours before the CD3 PEBL. In some cases, the vector encoding the CD3 PEBL may not be co-transduced with the vector encoding the CD3 CAR. In some cases, the vector encoding the CD3 CAR may not be transduced before the vector encoding the CD3 PEBL. When CD3 PEBL is transduced before the CD3 CAR, the CD3 CAR expression may be higher than the expression of CD3 CAR when the CD3 CAR is co-transduced with CD3 PEBL or when the CD3 CAR is transduced less than 36 hours (e.g., less than 35 hours, 34 hours, 33 hours, 32 hours, 31 hours, 30 hours, 29 hours, 28 hours, 27 hours, 26 hours, 25 hours, 24 hours, 23 hours, 22 hours, 21 hours, 20 hours, etc.) after transduction of the CD3 PEBL.

In some embodiments, the CD3 PEBL may be encoded by a polynucleotide (e.g., a first polynucleotide) and the CD3 CAR may be encoded by a polynucleotide (e.g., a second polynucleotide). The second polynucleotide may further comprise a kill gene as described herein. The immune cells may be activated prior to introducing the first polynucleotide into cells. Immune cells may be activated at least about 1 hour, at least about 2 hours, at least about 3 hours, at least about 6 hours, at least about 12 hours, at least about 1 day, at least about 2 days, at least about 3 days, or at least about 4 days prior to introduction of a first polynucleotide. Activation of the immune cells may comprise contacting the immune cells with an antibody. Activation of the immune cells may comprise contacting the immune cells with an anti-CD3 antibody and an anti-CD28 antibody.

The binding domain of the CD3 PEBL and CD3 CAR may be the same. The binding domain of the CD3 PEBL and CD3 CAR may be different. The binding domain of the CD3 PEBL and/or CD3 CAR of an immune cell or engineered immune cell as described herein may comprise a binding domain of a UCHT1 antibody, an OKT3 antibody, and/or a 28F11 antibody. The binding domain of the CD3 PEBL and/or CD3 CAR of an immune cell or engineered immune cell as described herein may comprise a VH domain comprising a sequence with at least about 90%, at least about 91%, at least about 92%, at least about 93%, at least about 94%, at least about 95%, at least about 96%, at least about 97%, at least about 98%, at least about 99%, or 100% sequence identity to an amino acid sequence as set forth in SEQ ID Nos: 1, 3, or 5. The binding domain or the CD3 PEBL and/or CD3 CAR of an immune cell or engineered immune cell as described herein may comprise a VL domain comprising a sequence with at least about 90%, at least about 91%, at least about 92%, at least about 93%, at least about 94%, at least about 95%, at least about 96%, at least about 97%, at least about 98%, at least about 99%, or 100% sequence identity to an amino acid sequence as set forth in SEQ ID Nos: 2, 4, or 6.

In some embodiments, the immune cells may further undergo culturing in a growth media. Culturing in the growth media may expand the cell population of immune cells (e.g., engineered immune cells). The cell population may expand at least about 2-fold, at least about 5-fold, at least about 10-fold, at least about 12-fold, at least about 20-fold, at least about 25-fold, at least about 30-fold, or at least about 40-fold after a period of growth (e.g., less than 20 days, less than 18 days, less than 15 days, less than 12 days, less than 10 days, less than 8 days, or less than 5 days of growth).

In some embodiments, the immune cell is a peripheral blood mononuclear cell, e.g., a T cell. In some embodiments, at least 10%, at least 20%, at least 30%, at least 40%, at least 50%, at least 60%, at least 70%, at least 80%, at least 90%, or about 100% of the cells of the immune cell population are CD4-positive T cells. In some embodiments, at least 10%, at least 20%, at least 30%, at least 40%, at least 50%, at least 60%, at least 70%, at least 80%, at least 90%, or about 100% of the cells of the immune cell population are CD8-positive T cells. In some embodiments, the immune cells are activated before the introducing a first polynucleotide into the immune cells. In some embodiments, activation comprises contacting the immune cells with an anti-CD3 antibody and an anti-CD28 antibody, e.g., contacting the immune cells with a polymeric nanomatrix conjugated to anti-CD3 antibodies and anti-CD28 antibodies.

In some embodiments, the method further comprises culturing the cells in a growth media. In some embodiments, the cell population expands at least 10-fold, at least 20-fold, at least 30-fold, at least 40-fold, at least 50-fold, at least 60-fold, at least 70-fold, at least 80-fold, at least 90-fold, or about 100-fold after 20 or less days of growth e.g., after 19 days, 18 days, 17 days, 16 days, 15 days, 14 days, 13 days, 12 days, 11 days, 10 days, 9 days, 8 days, 7 days 6 days of 5 days after the cells are activated. In some embodiments, culturing comprises contacting the cells to a gas permeable membrane. In some embodiments, culturing further comprises harvesting the cell population and cryopreserving the cell population.

In some embodiments, a cell population comprising the immune cells or engineered immune cells as described herein may comprise a number of cells of the cell population that are positive for a CAR and positive for CD20 (e.g., CAR+CD20+ cells). In some embodiments, at least about 10%, at least about 20%, at least about 30%, at least about 40%, at least about 50%, at least about 60%, at least about 70%, at least about 80%, at least about 90%, at least about 95%, at least about 99%, or 100% of cells of the cell population are CAR+CD20+ cells.

The CAR+CD20+ cells may be susceptible to antibody-dependent cellular cytotoxicity (ADCC) or complement dependent cytotoxicity (CDC). The CAR+CD20+ cells may be susceptible to ADCC or CDC by CD7 knockout effector cells expressing a chimeric receptor. The CAR+CD20+ cells may be susceptible to ADCC or CDC by NK effector cells expressing a chimeric receptor. For example, the effector cells can be NK-92MI cells. The chimeric receptor expressed by the NK-92MI effector cells may comprise an extracellular CD16 Fc binding domain, a transmembrane domain, and a cytoplasmic domain with a 4-1BB costimulatory domain and a CD3ζ primary signaling domain. The effector cells may comprise NK cells that do not express a chimeric receptor. The effector cells may comprise cells that have not been engineered. The effector cells may comprise primary NK cells. The effector cells to target cells ratio may be at least 1:1 during an incubation period (e.g., an at least 1 hour, 2 hour, 3 hours, 4 hours, 5 hour, 6 hour, 12 hour, 24 hour, 36 hour, 48 hour, 60 hour, or 72 hour incubation period) with rituximab. In some embodiments, the rituximab may be delivered at a dose of at least about 0.1 μg/ml, at least about 0.5 μg/ml, at least about 1 g/ml, at least about 1.5 μg/ml, at least about 2 μg/ml, at least about 2.5 μg/ml, at least about 3 μg/ml, at least about 3.5 μg/ml, at least about 4 μg/ml, at least about 4.5 μg/ml, at least about 5 μg/ml, at least about 10 μg/ml, at least about 12 μg/ml, at least about 15 μg/ml, at least about 18 μg/ml, at least about 20 μg/ml, or more.

In various aspects, also provided is a kit for producing an engineered immune cell described herein. The present kit can be used to produce, e.g., allogeneic or autologous T cells having CD3 CAR-mediated cytotoxic activity. In some embodiments, the kit is useful for producing allogeneic effector T cells having CD3 CAR-mediated cytotoxic activity. In certain embodiments, the kit is useful for producing autologous effector T cells having CD3 CAR-mediated cytotoxic activity.

Accordingly, provided herein is a kit comprising a nucleic acid comprising a nucleotide sequence encoding a CAR, wherein the CAR comprises intracellular signaling domains of 4-1BB and CD3ζ, and an antibody that binds CD3. The nucleotide sequence encoding the CD3 CAR can be designed according to any of the embodiments described herein. A kit may comprise a polynucleotide, a vector, an immune cell, an engineered immune cell, a cell population, a cellular composition, and/or a therapeutic composition as described herein.

In certain embodiments, the kit further comprises a nucleic acid having a nucleotide sequence that encodes a target-binding molecule linked to a localizing domain, as described herein (e.g., CD3 PEBL molecules described herein). The nucleotide sequence encoding the target-binding molecule linked to a localizing domain can be designed according to any of the embodiments described herein.

In certain embodiments, the nucleotide sequence encoding the CD3 CAR and/or the nucleotide sequence encoding the CD3 PEBL further comprise sequences (e.g., plasmid or vector sequences) that allow, e.g., cloning and/or expression. For example, the nucleotide sequence can be provided as part of a plasmid for ease of cloning into other plasmids and/or vectors (expression vectors or viral expression vectors) for, e.g., transfection, transduction, or electroporation into a cell (e.g., an immune cell). In certain embodiments, the nucleotide sequence encoding the CD3 CAR and the nucleotide sequence encoding the CD3 PEBL are provided on a single plasmid or vector (e.g., a single construct comprising a CD3 CAR and a CD3 PEBL). In certain embodiments, the nucleotide sequences are provided on separate plasmids or vectors (expression vectors or viral expression vectors).

Typically, the kits are compartmentalized for ease of use and can include one or more containers with reagents. In certain embodiments, all of the kit components are packaged together. Alternatively, one or more individual components of the kit can be provided in a separate package from the other kit components. The kits can also include instructions for using the kit components.

In some embodiments, provided herein is an engineered immune cell comprising: a first nucleic acid that comprises a first nucleotide sequence encoding a CD3 CAR linked to second nucleotide sequence encoding a CD20 protein, e.g., a truncated CD20 protein having an amino acid sequence of SEQ ID NO: 120 or SEQ ID NO: 121 as described herein and a second nucleic acid comprising a third nucleotide sequence encoding a target-binding molecule comprising an antibody or antigen binding fragment that specifically binds CD3 linked to a localizing domain. In some embodiments, the disclosure provides an engineered immune cell comprising: a first nucleic acid that comprises a nucleotide sequence encoding a chimeric antigen receptor (CAR), wherein the CAR comprises intracellular signaling domains of 4-1BB and CD3ζ, and an antibody or antigen binding fragment that specifically binds CD3 and a second nucleic acid comprising a nucleotide sequence encoding a target-binding molecule comprising an antibody or antigen binding fragment that specifically binds CD3 linked to a localizing domain.

In some embodiments, the CAR further comprises a hinge and transmembrane sequence, such as but not limited to a hinge and transmembrane domain comprising the amino acid sequence of SEQ ID NO:101.

In some embodiments, the engineered immune cell is an engineered T cell, an engineered natural killer (NK) cell, an engineered NK/T cell, an engineered monocyte, an engineered macrophage, or an engineered dendritic cell.

In some embodiments, the engineered immune cell further comprises a nucleic acid that comprises a nucleotide sequence encoding a target-binding molecule linked to an intracellular targeting signal. In certain embodiments, the target-binding molecule is an antibody that binds CD3. In certain embodiments, the antibody is an scFv. In some embodiments, the scFv comprises a VH sequence set forth in SEQ ID NO: 1 and a VL sequence set forth in SEQ ID NO: 2. In some embodiments, the intracellular targeting signal comprises an ER or Golgi retention sequence; a proteosome localizing sequence; a transmembrane domain sequence derived from TCRα, TCRβ, 4-1BB, CD28, CD34, CD4, FcεRIγ, CD16, OX40, CD3ζ, CD3ε, CD3γ, CD3δ, CD32, CD64, VEGFR2, FAS, or FGFR2B.

In some embodiments, the engineered immune cell further comprises a nucleic acid that comprises a nucleotide sequence encoding a target-binding molecule linked to an intracellular targeting signal wherein the intracellular targeting signal comprises an amino acid sequence as set forth in any one of the SEQ ID NOs: 137-143 or 147-149, or as set forth in any of the sequence identifiers A1 or A2. In some embodiments, the intracellular targeting signal comprises an ER retention sequence comprising an amino acid sequence as set forth in any one of SEQ ID NOs: 137-143. In some embodiments, the intracellular targeting signal comprises a Golgi retention sequence. In some embodiments, the Golgi retention sequence is any one of SEQ ID NOs: 147-149. In some embodiments, the intracellular targeting signal comprises a proteosome localizing sequence. In some embodiments, the proteosome localizing sequence comprises an amino acid sequence as set forth in SEQ ID NO: 150 or SEQ ID NO: 151.

In some embodiments, provided herein is an engineered immune cell comprising: a first nucleic acid that comprises a first nucleotide sequence encoding a CD3 CAR linked to second nucleotide sequence encoding a CD20 protein, e.g., a truncated CD20 protein having an amino acid sequence comprising SEQ ID NO: 120 or SEQ ID NO: 121 or a fragment thereof comprising a rituximab mimotope as described herein and a second nucleic acid comprising a third nucleotide sequence encoding a target-binding molecule comprising an antibody or antigen binding fragment that specifically binds CD3 linked to a localizing domain. In some embodiments, the disclosure provides an engineered immune cell comprising: a first nucleic acid that comprises a nucleotide sequence encoding a chimeric antigen receptor (CAR), wherein the CAR comprises intracellular signaling domains of 4-1BB and CD3ζ, and an antibody or antigen binding fragment that specifically binds CD3 and a second nucleic acid comprising a nucleotide sequence encoding a target-binding molecule comprising an antibody or antigen binding fragment that specifically binds CD3 linked to a localizing domain, and wherein the antibody that binds CD3 comprises a variable heavy chain (VH) sequence set forth in SEQ ID NO: 1 and a variable light chain (VL) sequence set forth in SEQ ID NO: 2. In some embodiments, the costimulatory domain of 4-1BB comprises the sequence set forth in SEQ ID NO: 102 and the primary signaling domain of CD3ζ comprises the sequence set forth in SEQ ID NO: 103.

In some embodiments, provided herein is a method of treating cancer in a subject in need thereof, comprising administering a therapeutic amount of the engineered immune cell described herein to the subject, thereby treating cancer in a subject in need thereof. In some embodiments, the cancer is a T cell malignancy. In one embodiment, the cancer is a T cell malignancy Peripheral T-Cell Lymphoma (PTCL). In certain embodiments, the engineered immune cell is administered into the subject by intravenous infusion, intra-arterial infusion, intraperitoneal infusion, direct injection into tumor and/or perfusion of tumor bed after surgery, implantation at a tumor site in an artificial scaffold, and/or intrathecal administration.

In some embodiments, provided herein is a method of treating an autoimmune disease in a subject in need thereof, comprising administering a therapeutic amount of the engineered immune cell described herein to the subject, thereby treating the autoimmune disease in a subject in need thereof. In some embodiments, the autoimmune disease is caused by an infection, e.g., a viral or bacterial infection. In some embodiments, the autoimmune disease is a severe autoimmune disease. In some embodiments, the autoimmune disease may include but are not limited to type I diabetes (T1D), multiple sclerosis (MS), rheumatoid arthritis (RA), inflammatory bowel diseases (IBDs), and myasthenia gravis (MG), systemic lupus erythematosus (SLE) and Sjögren's syndrome (SS).

In some embodiments, administering the engineered immune cell described herein may reduce a risk of developing an autoimmune disease or reduce the symptoms of an autoimmune disease in a subject in need thereof.

In some embodiments, provided herein is a nucleic acid comprising a nucleotide sequence encoding a chimeric antigen receptor (CAR), wherein the CAR comprises intracellular signaling domains of 4-1BB and CD3ζ, and an antibody that binds CD3 optionally wherein the nucleotide sequence further encodes a CD20, e.g., a truncated CD20.

In other embodiments, provided herein is the engineered immune cell described herein for treating cancer comprising administering a therapeutic amount of the engineered immune cell to a subject in need thereof. In some embodiments, the cancer is a T cell malignancy. In one embodiment, the cancer is a T cell malignancy Peripheral T-Cell Lymphoma (PTCL). In certain embodiments, the engineered immune cell is administered into the subject by intravenous infusion, intra-arterial infusion, intraperitoneal infusion, direct injection into tumor and/or perfusion of tumor bed after surgery, implantation at a tumor site in an artificial scaffold, and/or intrathecal administration.

In some embodiments, provided herein is a method for producing the engineered immune cell described herein. The method can include introducing into an immune cell a nucleic acid that comprises a nucleotide sequence encoding a CAR, wherein the CAR comprises intracellular signaling domains of 4-1BB and CD3ζ, and an antibody that binds CD3, thereby producing an engineered immune cell. The method can further comprise introducing into the immune cell a nucleic acid that comprises a nucleotide sequence encoding a target-binding molecule linked to a localizing domain.

The present disclosure provides a chimeric antigen receptor (CAR) directed against CD3. As demonstrated herein, the expression of the CD3 CAR in immune cells such as effector T cells, induces the T cell to exert specific cytotoxicity against T cell malignancies. This cytotoxic effect may be enhanced when expression of CD3 on the effector T cells is downregulated using an antibody-based molecule (a protein expression blocker or PEBL) that targeted the CD3 for downregulation. Thus, the present disclosure provides an immunotherapeutic method for treating cancers, e.g., T-cell malignancies.

In some aspects, the present disclosure provides an engineered immune cell (e.g., T cell, natural killer (NK) cell, NK/T cell, monocyte, macrophage, or dendritic cell) comprising (i) a nucleic acid that comprises a nucleotide sequence encoding a chimeric antigen receptor (CAR), wherein the CAR comprises intracellular signaling domains of 4-1BB and CD3ζ, and an antibody that specifically binds CD3; and (ii) a nucleic acid that comprises a nucleotide sequence encoding a target-binding molecule linked to a localizing domain, wherein the target-binding molecule is an antibody that binds CD3, and the localizing domain comprises an endoplasmic reticulum retention sequence, and wherein the antibody that binds CD3 comprises a variable heavy chain (VH) sequence set forth in SEQ ID NO: 1 and a variable light chain (VL) sequence set forth in SEQ ID NO: 2.

In other aspects, the present disclosure provides a method of treating cancer (e.g., a T cell malignancy) in a subject in need thereof. The method includes administering a therapeutic amount of any of the engineered immune cells described herein to the subject, thereby treating cancer in a subject in need thereof. The disclosure also sets forth the use of any of the engineered immune cells outlined herein for treating cancer.

### EXAMPLES

The following examples are provided to further illustrate some embodiments of the present dis-closure, but are not intended to limit the scope of the disclosure; it will be understood by their exemplary nature that other procedures, methodologies, or techniques known to those skilled in the art may alternatively be used.

The examples below are illustrative and non-limiting.

### Example 1: Generation of CD3 PEBL CD3 CAR T Cells by Sequential Transduction

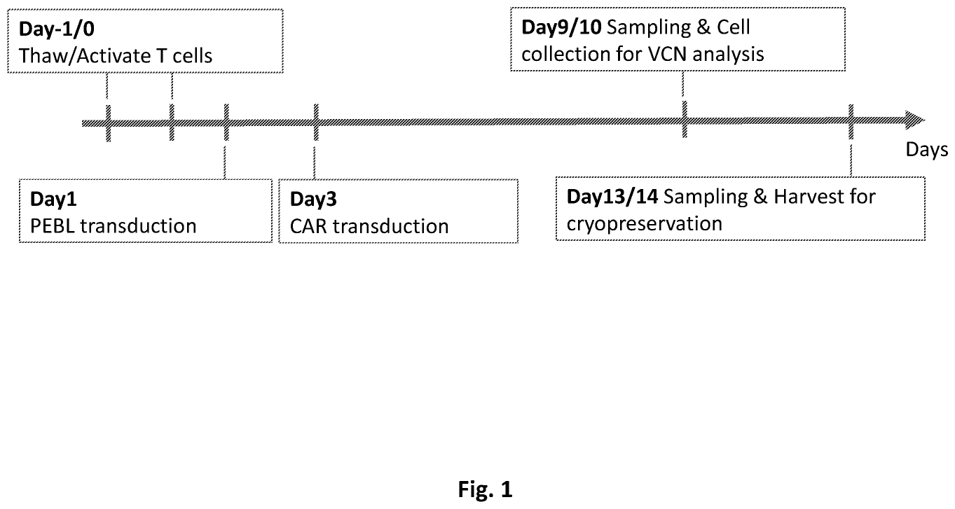

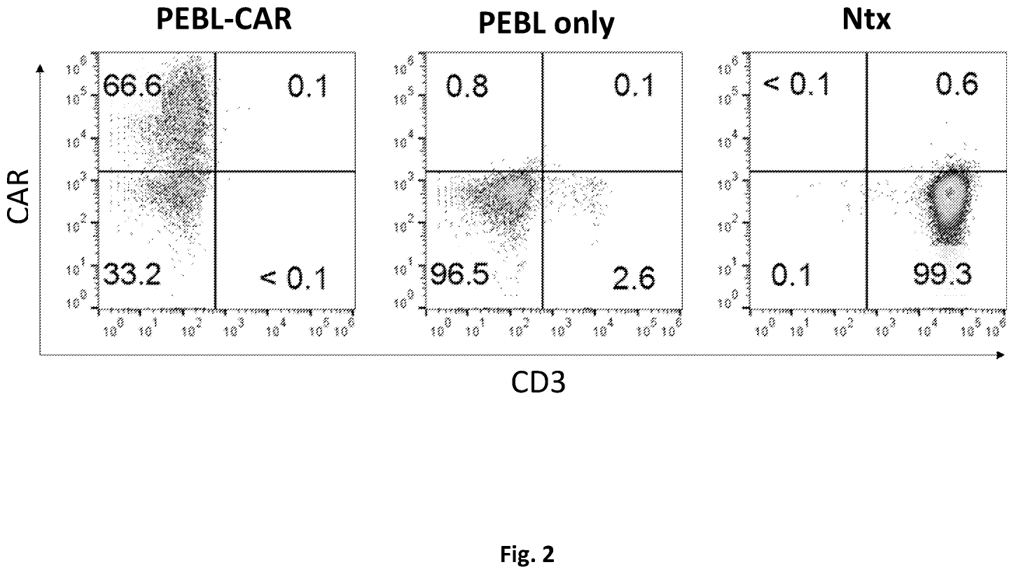

Human peripheral blood mononuclear cells (PBMC) or CD4+/CD8+ enriched T cells were thawed on Day −1 and activated by anti-CD3/CD28 antibodies on Day 0 (alternatively, thaw and activated on Day 0). Activated T cells were subsequently transduced with a lentiviral vector to express a CD3 PEBL comprising an scFv derived from the UCHT1 antibody on Day 1 and with a second lentiviral vector to express a CD3 CAR also comprising the UCHT1 scFv on Day 3. Sampling to determine the vector copy number (VCN) was performed on Day 9 or Day10. CD3 PEBL CD3 CAR T cells were harvested for cryopreservation on Day 13 or Day 14 (FIG. 1). A flow cytometry assay on Day9/10 cells shows that 66.6% of the transduced T cells expressed the CD3 CAR and had substantially reduced CD3 surface expression, indicating PEBL function (FIG. 2). The CD3 PEBL alone reduced CD3 surface expression in >95% of the T cells in comparison to a non-transduced (Ntx) control.

Sequential transduction of CD3 PEBL on Day 1 and CD3 CAR on Day 3 is a robust process yielding a high percentage of CAR positive cells after transduction of T cells from multiple donors. Results for CD3 CAR expression, growth, cytotoxicity against Jurkat (CD3+) target cells (24-hour assay, Effector:Target ratio 1:10) in CD3 PEBL CD3 CAR T cells generated from six healthy donors are shown in Table 13.

TABLE 13
Reproducibility of CD3 PEBL CD3 CAR T cells generated by sequential
transduction of PBMCs isolated from six donors.

| Donor | % CAR+ | Fold expansion | % Cytotoxicity |
|---|---|---|---|
| 1 | 30 | 95 | 65 |
| 2 | 55 | 48 | 72 |
| 3 | 35 | 42 | 65 |
| 4 | 42 | 11 | 63 |
| 5 | 49 | 44 | 65 |
| 6 | 62 | 59 | 65 |

In contrast, simultaneous expression of the CD3 PEBL and CD3 CAR yielded essentially no CAR positive T cells (Example 2), and sequential transduction on Day 1 and Day 2 yielded fewer CAR positive T cells (Example 4).

### Example 2: Simultaneous Transduction Failed to Generate CD3 PEBL CD3 CAR T Cells

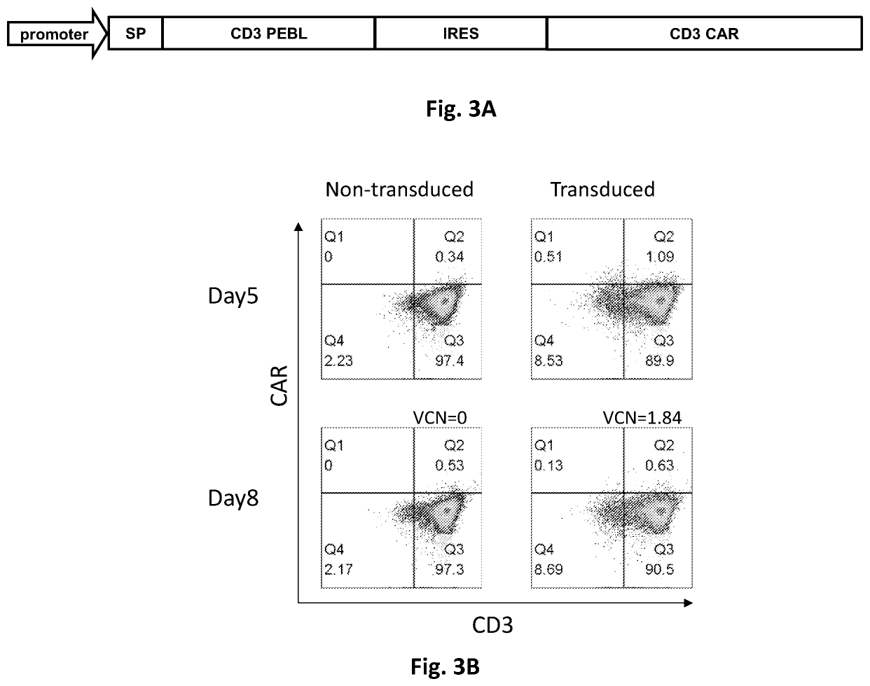

A bicistronic lentiviral vector for simultaneous expression of CD3 PEBL and CD3 CAR was constructed with an internal ribosome entry site (IRES) between sequences encoding the PEBL and CAR (FIG. 3A). Jurkat T cells transduced with the bicistronic vector and analyzed by flow cytometry had low CAR expression and high CD3 expression, similar to non-transduced cells. (FIG. 3B). Essentially no CAR positive and CD3 negative T cells were detected. Transduced cells were further analyzed for vector copy number (VCN), which confirmed that the cells had been successfully transduced.

### Example 3: Optimizing CD3 Binding Domains for CD3 PEBL and CD3 CAR Co-Expression

Many well-known antibodies bind to the TCR/CD3 complex, but each antibody has its own characteristics. Antibodies can have different complementarity determining regions in their binding domains, bind to different subunits of the complex, and bind to different epitopes of a subunit. scFvs derived from the OKT3, UCHT1, 28F11, and CIV3 antibodies were tested to identify functional combinations of antibodies for use in downregulating CD3 surface expression and activating T cell cytotoxicity against cells expressing CD3. For this comparison, T cells were activated on Day 0, transduced with PEBLs having different scFvs on Day 1, transduced with CARs having different scFvs on Day 3, and then analyzed by flow cytometry on Day 6.

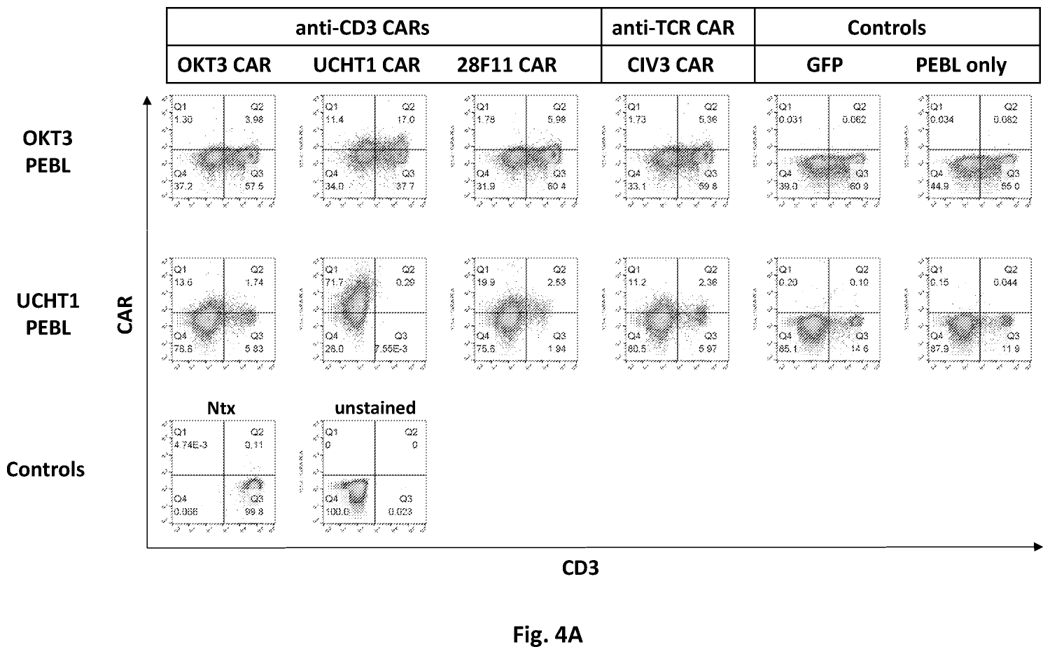

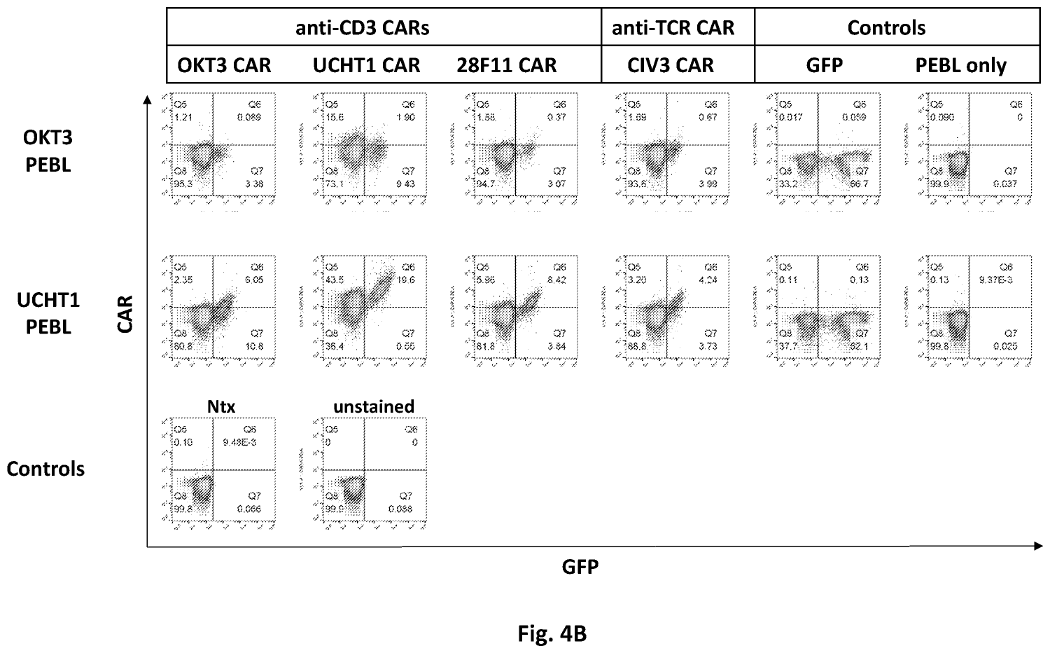

A CD3 PEBL comprising a UCHT1 scFv as the CD3 binding domain downregulated surface CD3 expression more effectively than a CD3 PEBL comprising a OKT3 scFv as the CD3 binding domain regardless of which scFv was incorporated into the CAR (FIG. 4A). Among the four CAR candidates bearing the indicated scFv, UCHT1 CAR exhibited the highest expression on Day 6 when coexpressed with UCHT1 PEBL (FIG. 4A). The CAR vectors used in this experiment were bicistronic and drove coexpression of GFP. Interestingly, some cells transduced with the OKT3 PEBL were GFP positive and CAR negative. Additionally, some cells transduced with the UCHT1 PEBL and OKT3 CAR were GFP-positive and CAR-negative, indicating that this combination resulted in poor surface expression of CAR (FIG. 4B).

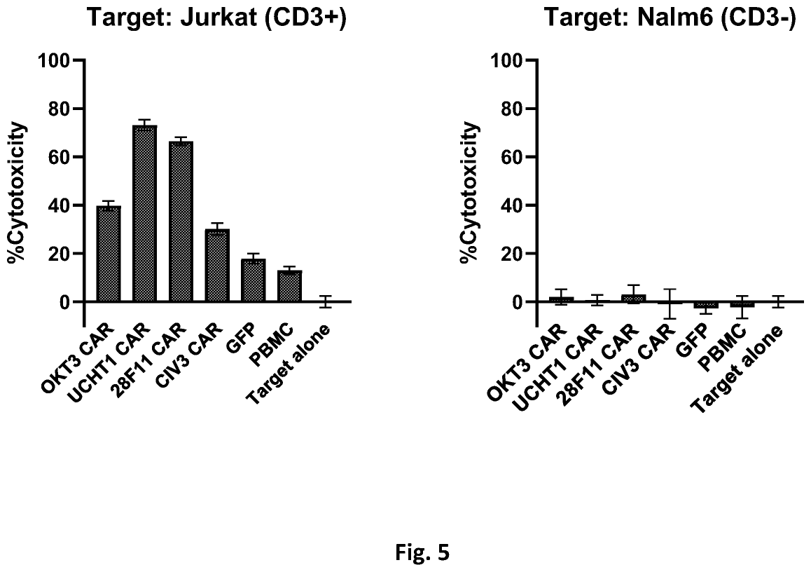

CAR function was tested in cytotoxicity assays against Jurkat (CD3+) and Nalm6 (CD3−) target cells expressing eGFP. The UCHT1 and 28F11 CARs in combination with the UCHT1 PEBL exhibited the highest cytotoxicity against Jurkat cells with little background non-specific cytotoxicity against Nalm6 cells (FIG. 5). The effector cells were generated by sequential transduction of PBMCs. Cytotoxicity was quantified over 24 hours at an effector-to-target ratio of 1:10.

### Example 4: Low CAR T Expression in T Cells Sequentially Transduced on Day 1 and Day 2

Viral transduction of T cells is more effective when the T cells are activated. The percentage of transduced cells obtained is highest if T cells are transduced one day after activation and then decreases over time. On the other hand, CD3 CAR expression causes fratricide in CD3 positive T cells. CD3 PEBL reduces CD3 surface expression, but this effect is not observed immediately after transduction. Instead, the CD3 PEBL accumulates over time after transduction. Thus, optimizing the transduction time is critical because the CAR will not be expressed well if it is transduced too late, while the CAR will cause excessive fratricide if it is transduced too early.

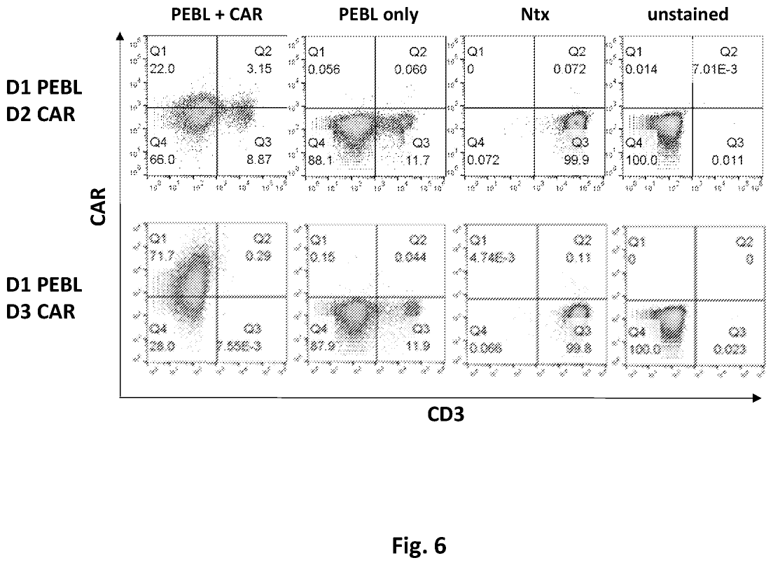

To determine the optimal time for CD3 CAR transduction, T cells were activated with anti-CD3 and anti-CD28 antibodies on Day 0, transduced with CD3 PEBL on Day 1, and then transduced with CD3 CAR on either Day 2 (D1D2) or Day 3 (D1D3). Flow cytometric analysis on Day 6 revealed that CAR expression was higher in the D1D3 cells (FIG. 6).

### Example 5: Cytotoxicity and Proliferation of CD3 PEBL-CAR T Cells in the Presence of CD3+ Target Cells In Vitro

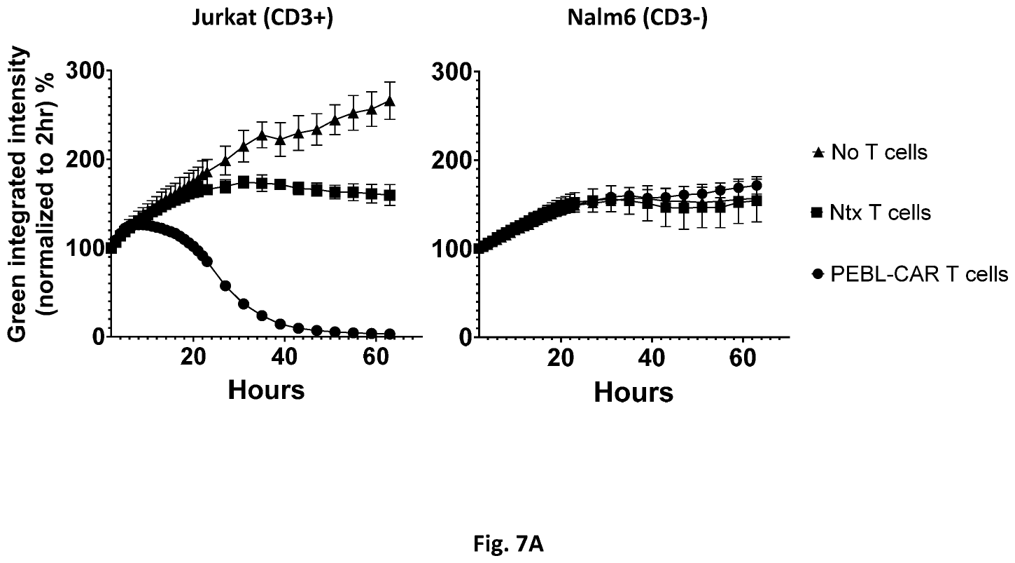

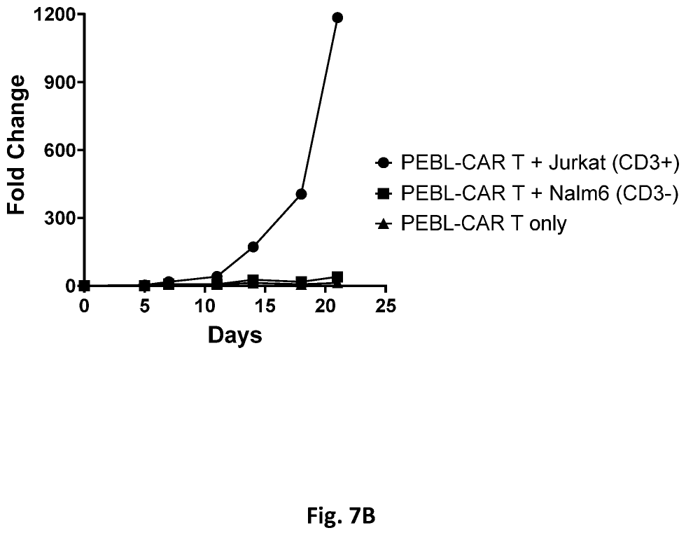

CD3 (UCHT1) PEBL CD3 (UCHT1) CAR T cells exhibited potent, specific, and long-term cytotoxicity against CD3 positive target cells (FIG. 7A). T cells generated by the method of Example 1 were cultured overnight at a 1:10 effector-to-target (E:T) ratio with Jurkat (CD3+) or Nalm6 (CD3−) target cells expressing eGFP. Viable target cell number was assessed over 60 hours with the IncuCyte live cell analysis system and represented as normalized integrated green fluorescence intensity.

In an assay for proliferation secondary to activation, the CD3 PEBL CD3 CAR T cells were cocultured with irradiated Jurkat (CD3+) or Nalm6 (CD3−) cells, with fresh irradiated target cells added at the start of each week, (day 0, 7, 14) to maintain stimulation of T cells when the cultures were replated. Growth was monitored over a period of 21 days. CD3 PEBL CD3 CAR T cells proliferated vigorously in the presence of CD3+ target cells and had substantially less growth in the presence of CD3− target cells (FIG. 7B).

### Example 6: Efficacy of CD3 PEBL CD3 CAR T Cells Against a CD3-Positive Tumor Xenograft

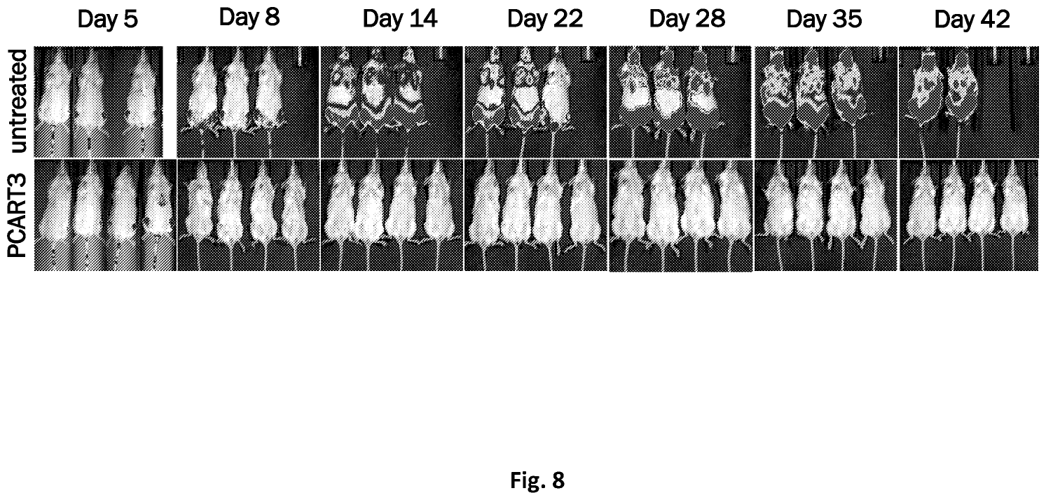

CD3 (UCHT1) PEBL CD3 (UCHT1) CAR T cells can eradicate T cell leukemia in an immunodeficient mouse model. NOD-SCID IL2RgammaNull (NSG) mice were infused intravenously with 5×10⁶ Jurkat cells expressing firefly luciferase and eGFP. Four days later, 2×10⁷ CD3 (UCHT1) PEBL CD3 (UCHT1) CAR T cells were administered intravenously to the treated group. Mice transplanted with Jurkat cells but not CD3 PEBL CAR T cells were used as a control. Bioluminescence images of the mice were taken on Days 5, 8, 14, 22, 28, 35, and 42 after infusion of the Jurkat cells. The mice exhibited a low bioluminescence signal on Day 5. Control NSG mice showed xenograft tumor growth over the time with fatalities by day 42. In contrast, no luciferase signal was detected in treated NSG mice after Day 8 and all mice survived for the full 42 days (FIG. 8).

### Example 7: CD3 PEBL Expression Prevents Graft Versus Host Disease

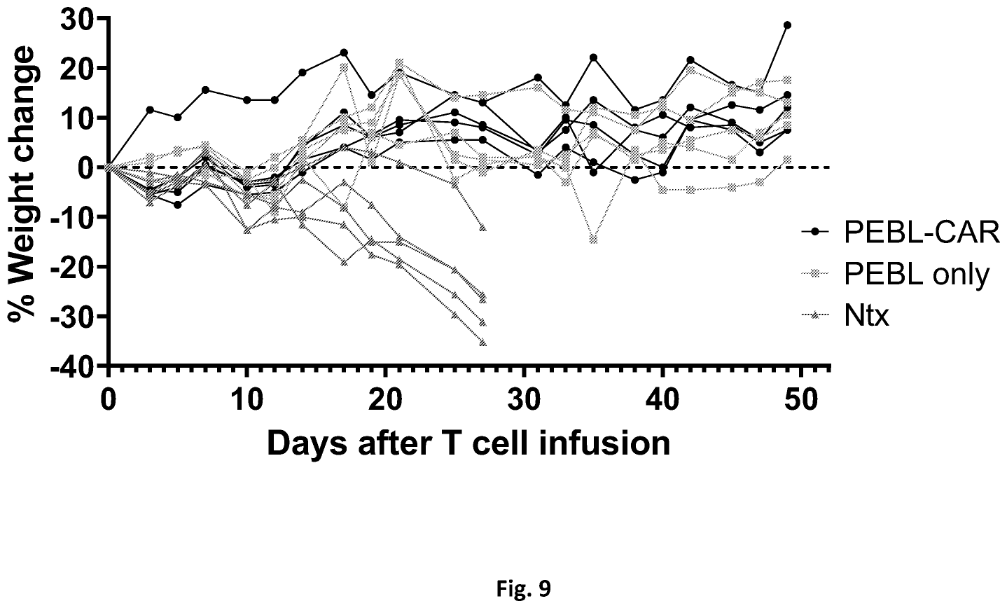

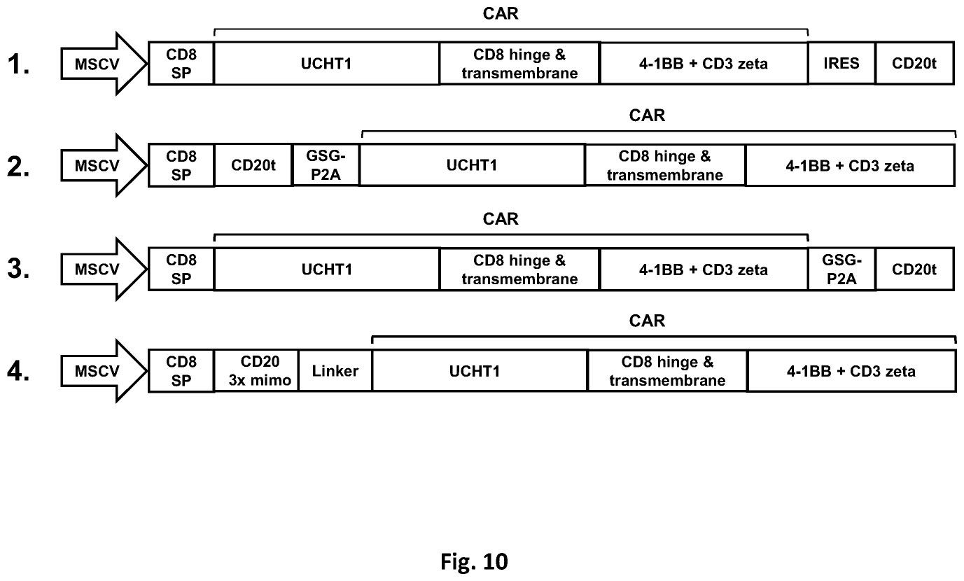

Transplantation of allogeneic T cells causes graft versus host disease (GvHD) when T cell receptors on the transplanted cells react with host antigens. To determine whether CD3 PEBL expression can prevent GvHD by downregulating TCR/CD3 surface expression, NSG mice were injected intravenously with 1×10⁷ allogenic T cells expressing or not expressing CD3 PEBL. To potentiate GvHD, the mice were irradiated with 2.5 Gy one day before the T cell injection. Significant weight loss indicative of GvHD was observed with parental xenogeneic T cells (Ntx). In contrast, mice transplanted with xenogeneic T cells expressing CD3 PEBL (with or without CD3 CAR co-expression) continued growing (FIG. 9). Thus, anti-CD3 CAR T cells coexpressing the CD3 PEBL did not mediate GvHD, indicating that the CD3 PEBL protects against GvHD.

### Example 8: Expression of a CD20-Based Kill Gene in CD3 PEBL CD3 CAR T Cells does not Interfere with CD3 PEBL Function

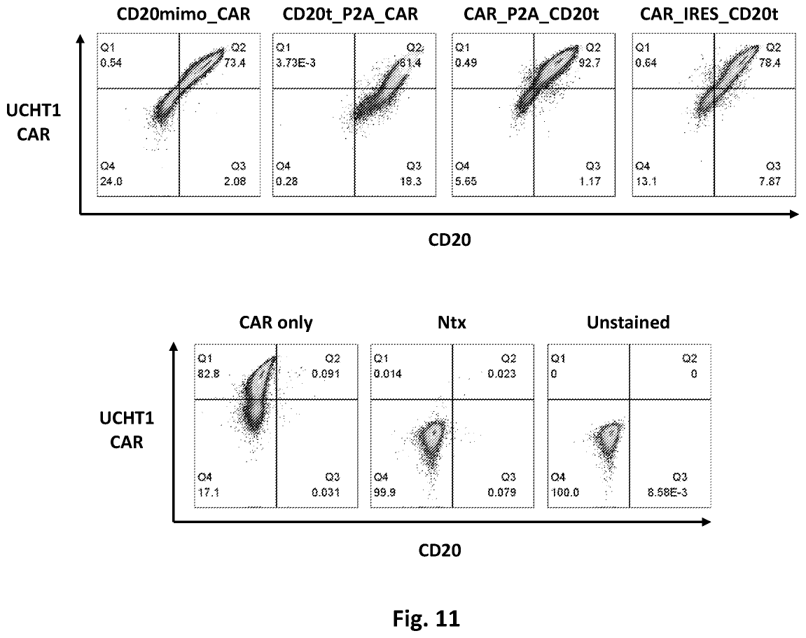

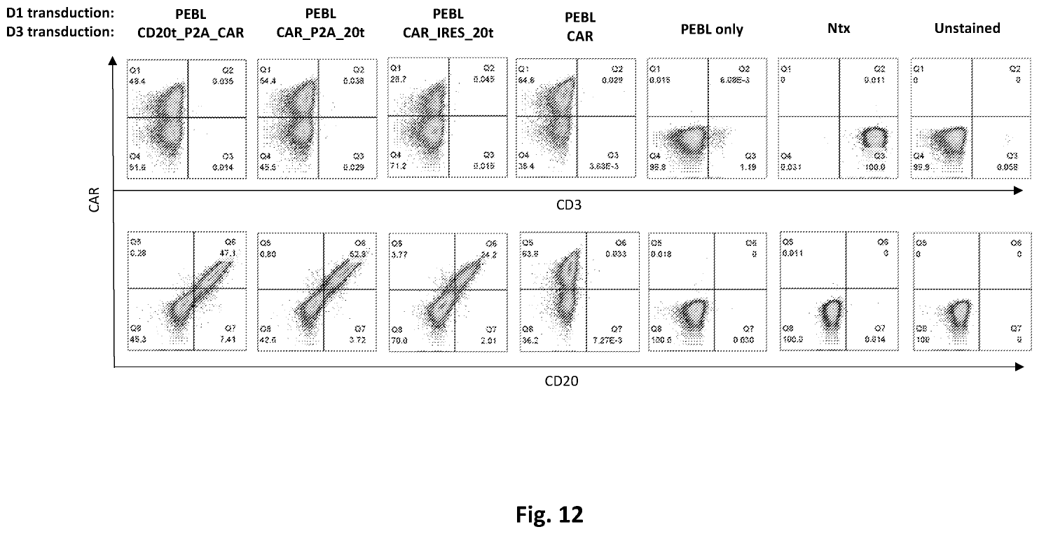

To enable rituximab mediated depletion of CAR-T cells as a safeguard should toxicity be experienced after CD3 CAR T cell treatment, four CAR-CD20 constructs were generated by incorporating a fragment of CD20 comprising the epitope for rituximab or mimotopes of the rituximab epitope into a UCHT1 CAR vector (FIG. 10). A positive correlation between CAR and rituximab staining was observed by flow cytometry for all four constructs when they were expressed in CD3 knockout Jurkat cells by lentiviral transduction. (FIG. 11). The CAR_P2A_CD20t construct yielded the highest percentage of CAR+CD20+ cells. This result indicates that all CAR-CD20t constructs are able to express the CD3 CAR and CD20 fragment and display the rituximab epitope on the cell surface. The three CAR-CD20t constructs were then compared to a CAR only construct. CD3 PEBL CD3 CAR T cells were generated according to the sequential transduction method of Example 1, and the cells were analyzed by flow cytometry on day 10 (FIG. 12). CD3 PEBL can downregulate CD3 expression fully in cells expressing CD20t. However, the CAR_IRES_CD20t construct yielded the lowest percentage of CAR+CD20+ cells while the CD20t_P2A_CAR construct gave lower CAR mean fluorescence intensity among CAR+ cells compared to the CAR_P2A_CD20t construct.

### Example 9: The CD20 Kill Gene does not Compromise CD3 CAR Function

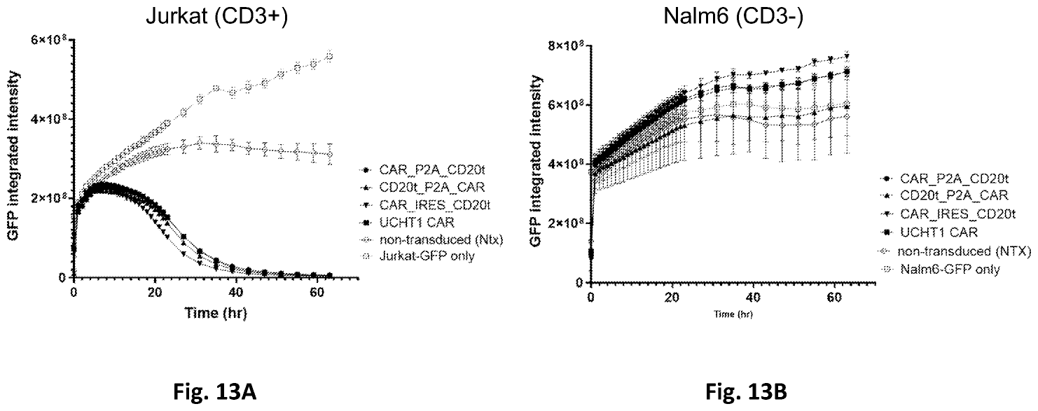

To investigate the effect of CD20t on CD3 CAR function, T cells sequentially transduced with CD3 PEBL and the three CAR-CD20t constructs were cultured overnight with Jurkat (CD3+) or Nalm6 (CD3−) target cells expressing eGFP at an effector:target (E:T) ratio of 1:10. Viable cell number was assessed over 60 hours with the IncuCyte live cell analysis system. All CAR-CD20t T cells exhibited potent cytotoxicity against CD3+ target cells when compared to non-transfected (Ntx) T cells. No non-specific killing of Nalm6 cells was observed (FIGS. 13A-13B).

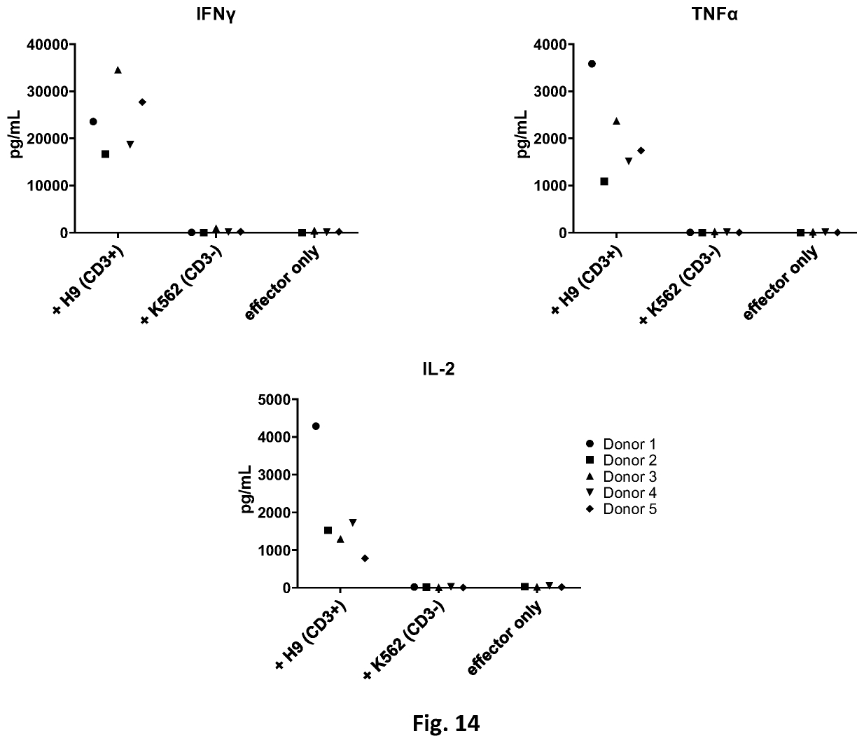

CD3 PEBL CD3 CAR T cells with CD20t kill gene manufactured from different donors were cocultured with target cells (CD3+), non-target cells (CD3−), or no target cells. Cells were cocultured for 18 hours. Levels of IFNγ, TNFα, and IL-2 in the co-culture supernatant were measured using LEGENDplex™ Human Th Cytokine Panel (BioLegend, cat #741028). CD3 PEBL CD3 CAR T cells with CD20t kill gene showed higher levels of IFNγ, TNFα, and IL-2 secretion when cultured with CD3+ cells, compared to levels when cultured with CD3− cells or no target cells (FIG. 14).

### Example 10: CD3 PEBL CD3 CAR T Cells Expressing CD20t are Sensitive to Rituximab Mediated Cytolysis

Cytotoxicity assays were used to determine whether coexpression of the CD20t kill gene can render CD3 PEBL CD3 CAR T cells susceptible to killing upon administration of rituximab, an anti-CD20 antibody.

To test for susceptibility to antibody-dependent cellular cytotoxicity (ADCC), CD3 PEBL CD3 CAR-CD20t T cells (e.g., PCART3^{KG} cells) were cocultured for 48 hours with anti-CD20 antibody and NK effector cells at various effector to target (E:T) ratios. The T cells were generated by sequential transduction with the CD3 PEBL and CD3 CAR_P2A_CD20t vectors. The anti-CD20 antibody was rituximab. Trastuzumab, an anti-Her2 antibody was used as a negative control. The NK effector cells were CD7 knockout NK-92MI cells expressing a chimeric receptor with an extracellular CD16 Fc receptor binding domain, a transmembrane domain, and a cytoplasmic domain with a 4-1BB costimulatory domain and a CD3ζ primary signaling domain.

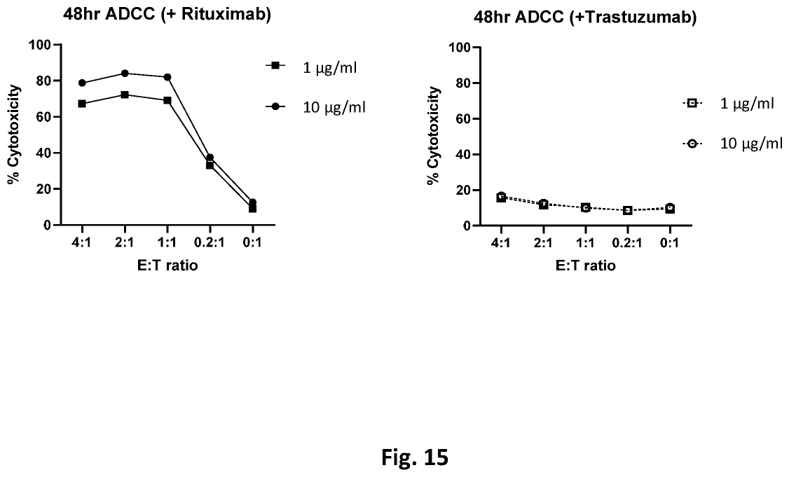

Dose-dependent killing of PCART3^{KG} cells was observed in the presence of rituximab, but not in the presence of trastuzumab, indicating that the CD20t kill gene rendered the T cells susceptible to ADCC (FIG. 15).

To test for susceptibility to complement-dependent cytotoxicity (CDC), PCART3^{KG} cells were incubated for 6 hours with 25% (v/v) baby rabbit complement and various concentrations of rituximab (anti-CD20) or trastuzumab (anti-Her2) antibodies.

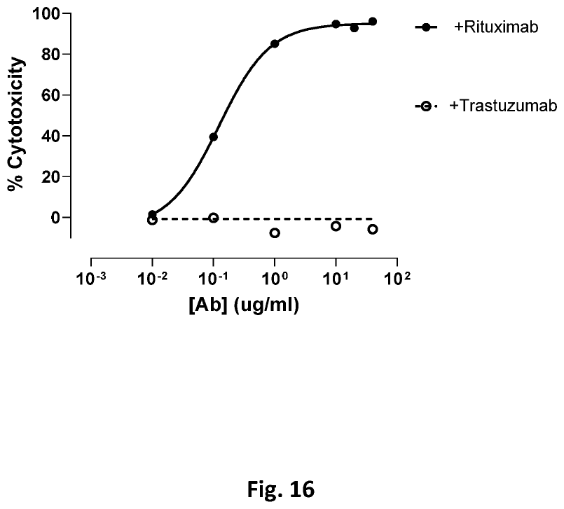

Dose-dependent killing of the PCART3^{KG} cells was observed in the presence of rituximab, but not in the presence of trastuzumab, indicating that the CD20t kill gene rendered the T cells susceptible to CDC (FIG. 16).

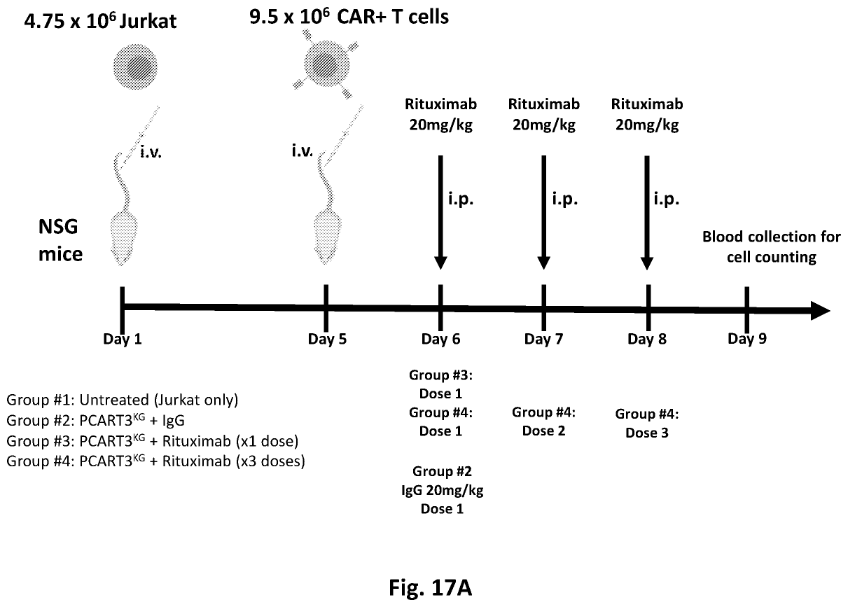

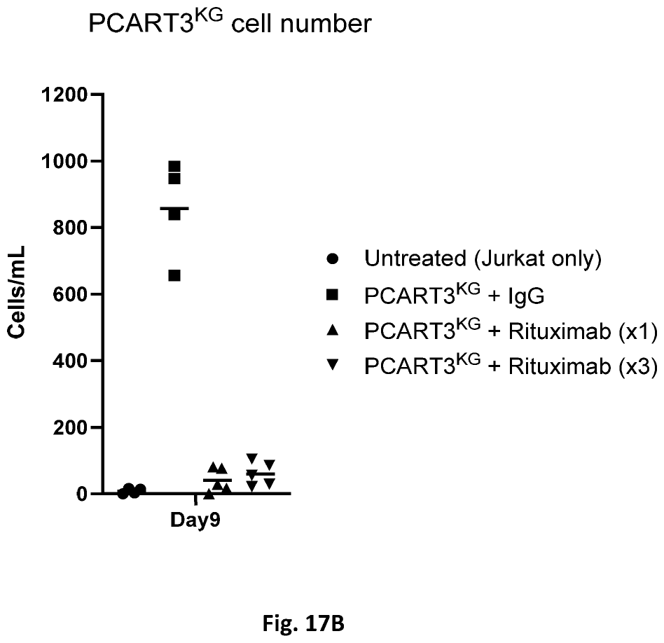

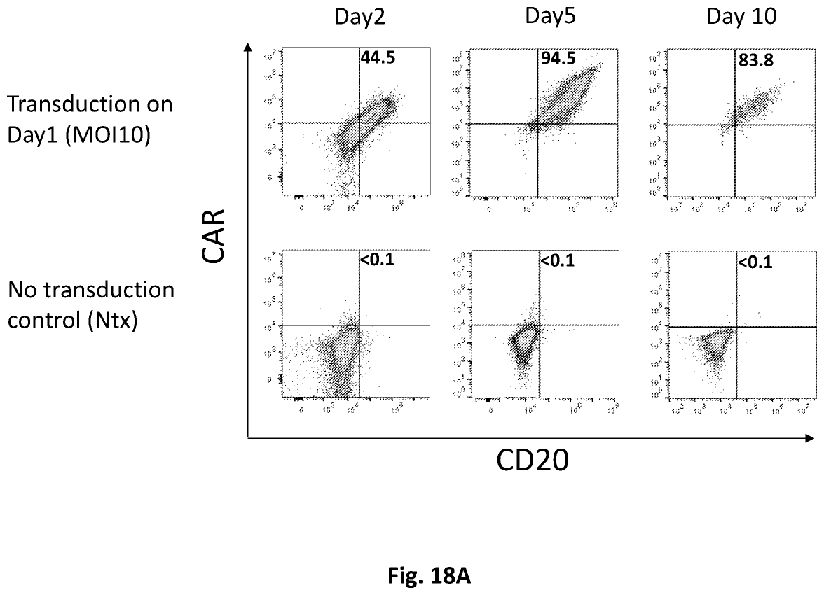

To test for effectiveness of rituximab to deplete PCART3^{KG} cells in vivo, NOD scid gamma (NSG) mice were intravenously injected with Jurkat cells on Day 1, followed by PCART3^{KG} cells on Day 5. There were four treatment groups: untreated (Jurkat cells only), PCART3^{KG} cells with one dose of IgG, PCART3^{KG} cells with one dose of rituximab, and PCART3^{KG N} cells with three doses of rituximab. 20 mg/kg rituximab was administered by intraperitoneal injection according to the dosing schedule as shown in FIG. 17A.

On Day 1, NSG mice were intravenously injected with 4.75×10⁶ Jurkat cells expressing firefly luciferase and eGFP. On Day 5, the mice were infused with 9.5×10⁶ PCART3^{KG} cells or left untreated. On Day 6, the mice treated with CD3 PEBL CD3 CAR T cells expressing CD20t, 20 mg of Rituximab or control human IgG was injected intraperitoneally. For the group receiving three total doses of rituximab, mice were injected daily for three days, beginning on Day 6. Following completion of dose administration, blood samples were collected from each mouse on Day 9. The number of PCART3^{KG} cells per mL were quantified by flow cytometry. Both the groups receiving one dose of rituximab or three doses of rituximab showed decreased PCART3^{KG} cell number compared to the group which received one dose of control human IgG (FIG. 17B). This demonstrated that rituximab depleted PCART3^{KG} cells in vivo.

### Example 11: Expression of CD3 CAR T Cells in the Absence of CD3 PEBL Leads to Fratricide

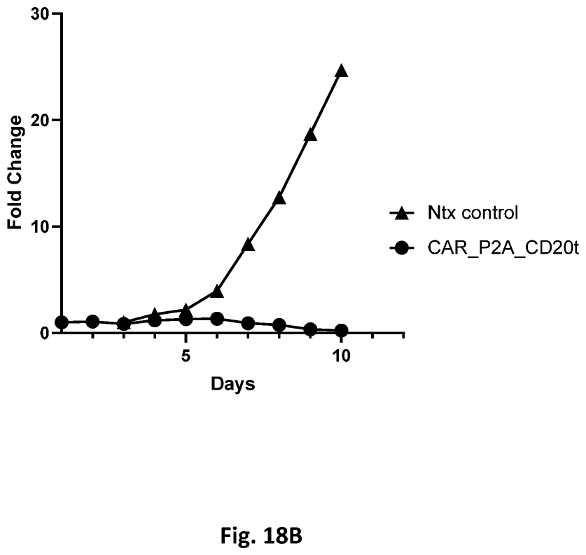

To test the effect of expressing CD3 CAR T cells without anti-CD3 PEBL, T cells were activated with TransAct™ on Day 0 and transduced with anti-CD3 CAR (with CD20t) lentiviral vector on Day 1 at MOI 10. There was also a control of non-transduced T cells (Ntx). Flow cytometric analysis of the anti-CD3 CAR transduced cells showed high levels of CAR and CD20t early after transduction on Day 5 (FIG. 18A). Expression had decreased by Day 10 post-transduction. Non-transduced T cells showed a lack of CAR or CD20t expression. Daily cell counts were also performed using flow cytometry and showed that the transduced anti-CD3 CAR-T cells did not proliferate (FIG. 18B). In contrast, the non-transduced T cells showed greater than 20-fold expansion across the 10 days of manufacturing.

### Example 12: In Vivo Xenoreactivity and Anti-Tumor Efficacy of PCART3KG Cells

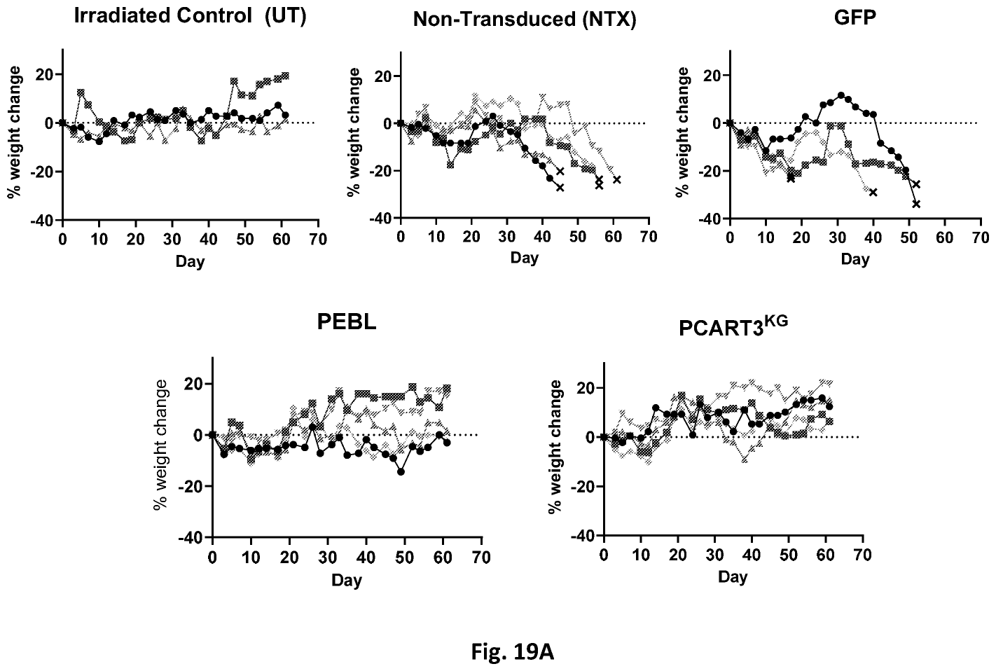

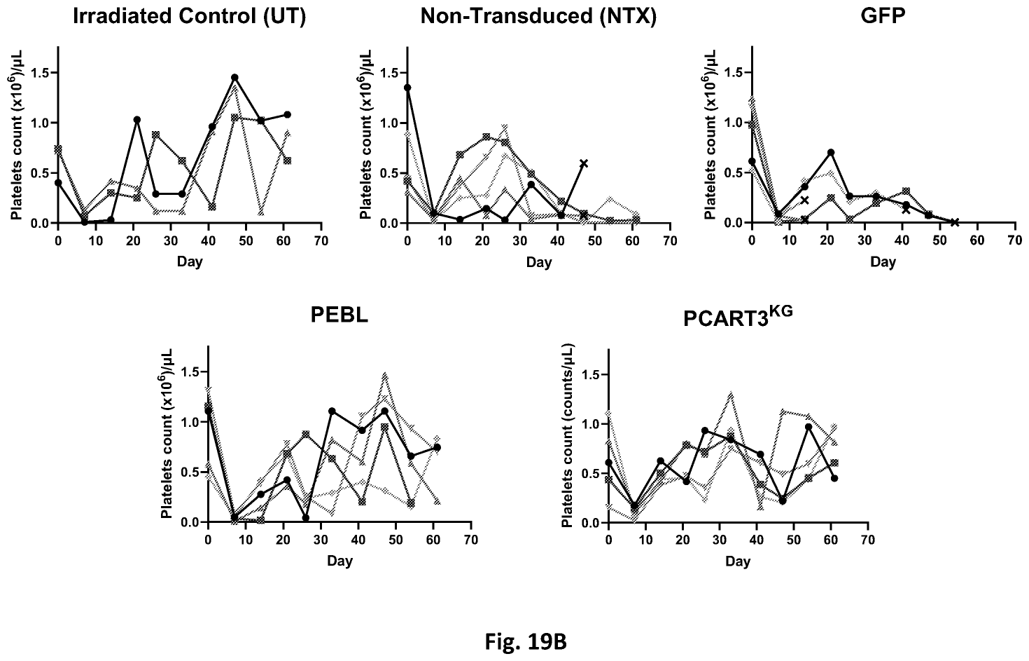

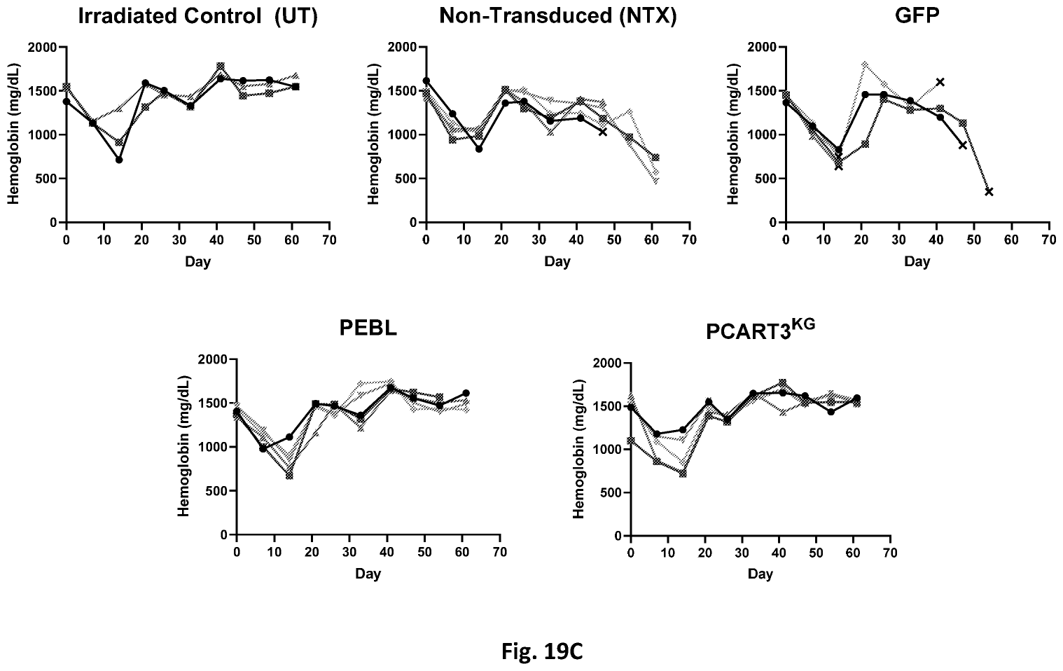

To test for whether PCART3^{KG} cells mediate xenoreactivity in vivo, NSG mice were first irradiated with 2.5 Gy then left untreated or injected intravenously with 2×10⁷ non-transduced or transduced T cells. There were five treatment groups: untreated irradiated control, non-transduced T cells, T cells transduced with eGFP only, T cells transduced with anti-CD3 PEBL only, or PCART3^{KG} cells transduced with CD3 PEBL, CD3 CAR and CD20t. Mice were monitored over time and body weight, peripheral blood platelet count and hemoglobin levels were quantified. Mice that received non-transduced T cells or T cells transduced with eGFP only showed significant weight loss of greater than 20% (FIG. 19A). Mice that received PCART3^{KG} cells did not show significant percentage change in weight. Similarly, mice that received PCART3^{KG} cells did not show decreasing platelet counts or hemoglobin levels over time, whereas mice that received non-transduced T cells or T cells transduced with eGFP only showed reduced platelet counts and hemoglobin levels (FIGS. 19B-C). Overall, the data indicate that PCART3^{KG} cells did not mediate xenoreactivity.

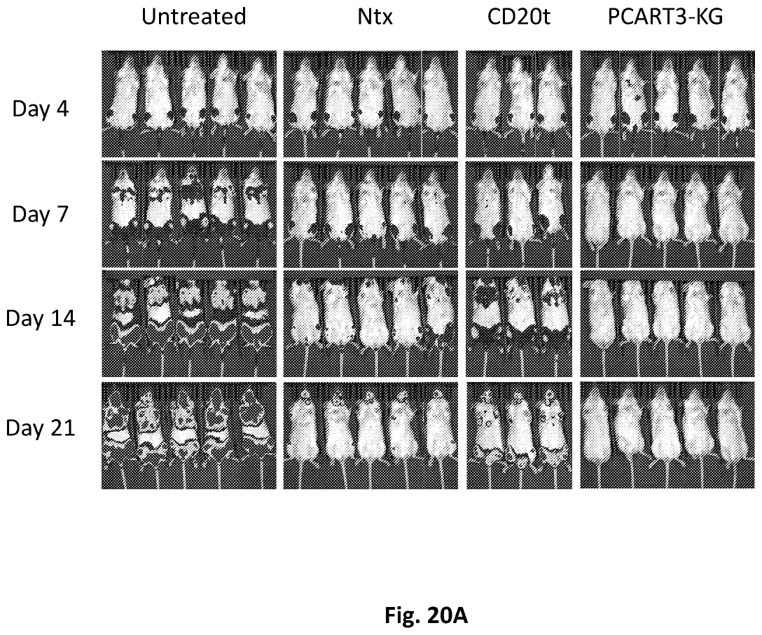

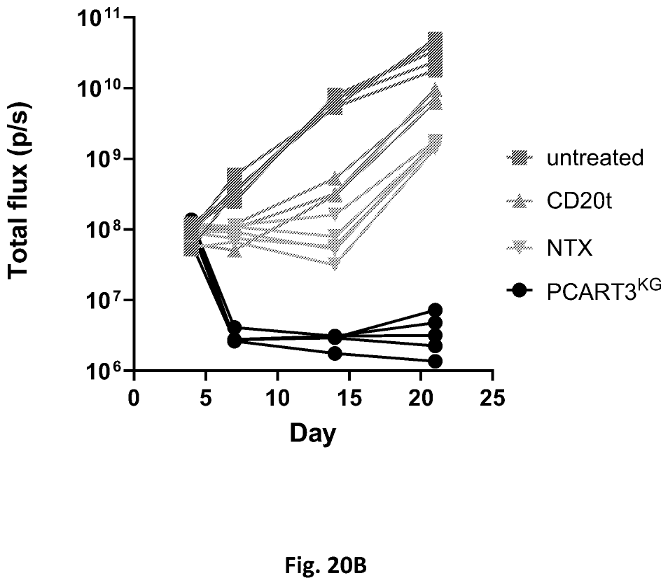

To test the effects of PCART3^{KG} cells on tumor killing in vivo, NSG mice were infused intravenously with 4×10⁶ Jurkat cells expressing firefly luciferase and eGFP on Day 0, followed by 2×10⁷ non-transduced or transduced T cells on Day 4. There were four treatment groups: an untreated control, non-transduced T cells, T cells transduced with CD20t only, or PCART3^{KG} cells transduced with CD3 PEBL, CD3 CAR and CD20t. Tumor growth was visualized using bioluminescent imaging on Days 4, 7, 14, 21 post tumor cell infusion (FIG. 20A). Analysis of total flux (in photons per second) from the bioluminescence showed the changes in tumor burden over time for each mouse. As shown in FIG. 20B, mice treated with PCART3^{KG} cells showed lower total flux values than those mice in the untreated control, non-transduced, and CD20t only groups, demonstrating superior tumor killing activity of PCART3^{KG} cells.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. An immune cell comprising:
a first polynucleotide comprising a nucleotide sequence encoding a protein expression blocker (PEBL) and
a second polynucleotide comprising a nucleotide sequence encoding a chimeric antigen receptor (CAR),
wherein the PEBL comprises a first CD3-binding domain coupled to an intracellular targeting signal,
wherein the intracellular targeting signal comprises an endoplasmic reticulum (ER) retention sequence, a Golgi retention sequence, or a proteosome localizing sequence,
wherein the PEBL downregulates cell surface expression of endogenous CD3 in the immune cell,
wherein the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif,
wherein the first CD3-binding domain and the second CD3-binding domain each comprises six complementarity determining regions (CDRs) from heavy chain (HC) and light chain (LC) variable domains of a UCHT1 antibody, and wherein:
HC CDR1 comprises SEQ ID NO: 13 or SEQ ID NO: 31,
HC CDR2 comprises SEQ ID NO: 14 or SEQ ID NO: 32,
HC CDR3 comprises SEQ ID NO: 15 or SEQ ID NO: 33,
LC CDR1 comprises SEQ ID NO: 22 or SEQ ID NO: 40,
LC CDR2 comprises SEQ ID NO: 23 or SEQ ID NO: 41, and
LC CDR3 comprises SEQ ID NO: 24 or SEQ ID NO: 42.

2. The immune cell of claim 1, wherein the first CD3-binding domain and the second CD3-binding domain each comprises a heavy chain variable (VH) domain comprising a sequence having at least 90% identity to SEQ ID NO: 1 and a light chain variable (VL) domain comprising a sequence having at least 90% identity to SEQ ID NO: 2.

3. The immune cell of claim 1, wherein the first CD3-binding domain and the second CD3-binding domain each comprises a VH domain comprising a sequence of SEQ ID NO: 1 and a VL domain comprising a sequence of SEQ ID NO: 2.

4. An immune cell comprising:
a first polynucleotide comprising a nucleotide sequence encoding a protein expression blocker (PEBL) and
a second polynucleotide comprising a nucleotide sequence encoding a chimeric antigen receptor (CAR),
wherein the PEBL comprises a first CD3-binding domain coupled to an intracellular targeting signal,
wherein the intracellular targeting signal comprises an endoplasmic reticulum (ER) retention sequence, a Golgi retention sequence, or a proteosome localizing sequence,
wherein the PEBL downregulates cell surface expression of endogenous CD3 in the immune cell,
wherein the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif,
wherein the first CD3-binding domain comprises six complementarity determining regions (CDRs) from heavy chain (HC) and light chain (LC) variable domains of a UCHT1 antibody, wherein:
HC CDR1 comprises SEQ ID NO: 13 or SEQ ID NO: 31,
HC CDR2 comprises SEQ ID NO: 14 or SEQ ID NO: 32,
HC CDR3 comprises SEQ ID NO: 15 or SEQ ID NO: 33,
LC CDR1 comprises SEQ ID NO: 22 or SEQ ID NO: 40,
LC CDR2 comprises SEQ ID NO: 23 or SEQ ID NO: 41, and
LC CDR3 comprises SEQ ID NO: 24 or SEQ ID NO: 42; and
wherein the second CD3-binding domain comprises six complementarity determining regions (CDRs) from heavy chain (HC) and light chain (LC) variable domains of a 28F11 antibody, wherein:
HC CDR1 comprises SEQ ID NO: 19 or SEQ ID NO: 37,
HC CDR2 comprises SEQ ID NO: 20 or SEQ ID NO: 38,
HC CDR3 comprises SEQ ID NO: 21 or SEQ ID NO: 39,
LC CDR1 comprises SEQ ID NO: 28 or SEQ ID NO: 46,
LC CDR2 comprises SEQ ID NO: 29 or SEQ ID NO: 47, and
LC CDR3 comprises SEQ ID NO: 30 or SEQ ID NO: 48.

5. The immune cell of claim 4, wherein the first CD3-binding domain and the second CD3-binding domain each comprises a heavy chain variable (VH) domain comprising a sequence having at least 90% identity to SEQ ID NO: 5 and a light chain variable (VL) domain comprising a sequence having at least 90% identity to SEQ ID NO: 6.

6. The immune cell of claim 4, wherein the first CD3-binding domain and the second CD3-binding domain each comprises a VH domain comprising a sequence of SEQ ID NO: 5 and a VL domain comprising a sequence of SEQ ID NO: 6.

7. The immune cell of any one of claims 1-6, wherein the PEBL further comprises a linker sequence between the first CD3-binding domain and the intracellular targeting signal.

8. The immune cell of claim 7, wherein the linker sequence comprises a sequence of SEQ ID NO: 135 or SEQ ID NO: 139.

9. The immune cell of any one of claims 1-8, wherein the ER retention sequence comprises KDEL (SEQ ID NO: 137), KKXX or KXD/E, wherein X is any amino acid.

10. The immune cell of claim 9, wherein the ER retention sequence comprises SEQ ID NO: 142.

11. The immune cell of claim 9, wherein the ER retention sequence comprises SEQ ID NO: 143.

12. The immune cell of any one of claims 1-11, wherein the Golgi retention sequence comprises YQRL (SEQ ID NO: 147).

13. The immune cell of any one of claims 1-12, wherein the transmembrane domain comprises SEQ ID NO: 100.

14. The immune cell of any one of claims 1-13, wherein the costimulatory domain comprises SEQ ID NO: 102.

15. The immune cell of any one of claims 1-14, wherein the cytoplasmic signaling domain comprises SEQ ID NO: 103.

16. The immune cell of any one of claims 1-15, wherein the second polynucleotide further comprises a kill gene.

17. The immune cell of claim 16, wherein the kill gene comprises a sequence encoding CD20 or a truncated fragment thereof.

18. The immune cell of any one of claims 16-17, wherein the CD20 fragment comprises SEQ ID NO: 124.

19. The immune cell of any one of claims 16-18, wherein the second polynucleotide comprises an internal ribosome entry site between the nucleotide sequence encoding the CAR and the sequence encoding the kill gene.

20. The immune cell of any one of claims 16-18, wherein the second polynucleotide comprises a nucleotide sequence encoding a self-cleaving peptide between the nucleotide sequence encoding the CAR and the sequence encoding the kill gene.

21. The immune cell of claim 20, wherein the second polynucleotide comprises, in the 5′ to 3′ direction, the sequence encoding the CAR, the sequence encoding a self-cleaving peptide, and the sequence encoding a truncated CD20 fragment.

22. The immune cell of any one of claims 20-21, wherein the self-cleaving peptide comprises a P2A sequence.

23. The immune cell of claim 22, wherein the self-cleaving peptide further comprises a linker sequence.

24. The immune cell of claim 23, wherein the linker sequence comprises GSG.

25. The immune cell of any one of claims 20-24, wherein the self-cleaving peptide comprises SEQ ID NO: 122.

26. The immune cell of any one of claims 1-15, wherein the immune cell further comprises a third polynucleotide further comprises a kill gene.

27. The immune cell of claim 26, wherein the kill gene comprises a sequence encoding CD20 or a truncated fragment thereof.

28. The immune cell of any one of claims 26-27, wherein the CD20 fragment comprises SEQ ID NO: 124.

29. The immune cell of any one of claims 1-28, wherein the first polynucleotide further comprises a nucleotide sequence encoding a CD8 signal peptide operably linked to the nucleotide sequence encoding the PEBL.

30. The immune cell of any one of claims 1-29, wherein the first polynucleotide further comprises an MSCV promoter operably linked to the nucleotide sequence encoding the PEBL.

31. The immune cell of any one of claims 1-30, wherein the second polynucleotide further comprises an MSCV promoter operably linked to the nucleotide sequence encoding the CAR.

32. The immune cell of any one of claims 1-31, wherein the nucleotide sequence encoding a PEBL comprises a codon optimized sequence.

33. An immune cell comprising:
a first polynucleotide comprising a nucleotide sequence encoding a protein expression blocker (PEBL) and
a second polynucleotide comprising a nucleotide sequence encoding a chimeric antigen receptor (CAR) and a nucleotide sequence encoding a kill gene,
wherein the PEBL comprises a first CD3-binding domain coupled to an intracellular targeting signal,
wherein the intracellular targeting signal comprises an endoplasmic reticulum (ER) retention sequence, a Golgi retention sequence, or a proteosome localizing sequence, and
wherein the PEBL downregulates cell surface expression of endogenous CD3 in the immune cell, and
wherein the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif.

34. The immune cell of claim 33, wherein the kill gene comprises a sequence encoding CD20 or a truncated fragment thereof.

35. The immune cell of claim 34, wherein the CD20 fragment comprises SEQ ID NO: 124.

36. The immune cell of any one of claims 33-35, wherein the second polynucleotide comprises an internal ribosome entry site between the nucleotide sequence encoding the CAR and the kill gene.

37. The immune cell of any one of claims 33-35, wherein the second polynucleotide comprises a nucleotide sequence encoding a self-cleaving peptide between the nucleotide sequence encoding the CAR and the nucleotide sequence encoding the kill gene.

38. The immune cell of claim 37, wherein the second polynucleotide comprises, in the 5′ to 3′ direction, the sequence encoding the CAR, the sequence encoding a self-cleaving peptide, and the sequence encoding a truncated CD20 fragment.

39. The immune cell of any one of claims 37-38, wherein the self-cleaving peptide comprises GSG-P2A.

40. The immune cell of any one of claims 37-38, wherein the self-cleaving peptide comprises SEQ ID NO: 122.

41. The immune cell of any one of claims 33-40, wherein the first CD3-binding domain and the second CD3-binding domain are the same CD3-binding domain.

42. The immune cell of any one of claims 1-41, wherein the immune cell secretes one or more cytokines in the presence of CD3+ cells.

43. The immune cell of claim 42, wherein the one or more cytokines comprises IFNγ, TNFα, and/or IL-2.

44. The immune cell of any one of claims 1-43, wherein the immune cell does not mediate xenoreactivity when administered into a subject in need thereof.

45. The immune cell of any one of claims 1-43, wherein the immune cell does not mediate alloreactivity when administered into a subject in need thereof.

46. A cell population comprising the immune cell of any one of claims 1-45.

47. The cell population of claim 46, wherein the cell population comprises peripheral blood mononuclear cells.

48. The cell population of claim 46 or 47, wherein at least 80% of the cells of the cell population are T cells.

49. The cell population of any one of claims 46-48, wherein at least 40% of the cells of the cell population are CD4-positive T cells.

50. The cell population of any one of claims 46-49, wherein at least 40% of the cells of the cell population are CD8-positive T cells.

51. The cell population of any one of claims 46-50, wherein at least 80% of cells of the cell population have at least 10-fold reduced cell surface expression of CD3 compared to an otherwise identical cell population that does not comprise an immune cell comprising the first polynucleotide encoding the PEBL.

52. The cell population of any one of claims 46-51, wherein at least 30% of cells of the cell population express the CAR.

53. The cell population of any one of claims 46-52, wherein at least 50% of cells of the cell population have at least 10-fold reduced cell surface expression of CD3 compared to an otherwise identical cell population that does not comprise an immune cell comprising the first polynucleotide encoding the PEBL and express the CAR.

54. The cell population of any one of claims 46-53, wherein the cell population is capable of at least 10-fold expansion in 10 days.

55. The cell population of any one of claims 46-54, wherein the cells of the cell population having at least 10-fold reduced cell surface expression of CD3 compared to an otherwise identical cell population that does not comprise an immune cell comprising the first polynucleotide encoding the PEBL and expressing the CAR are capable of at least 20-fold expansion in 10 days.

56. The cell population of any one of claims 46-55, wherein at least 20% of the cells of the cell population are CAR+CD20+ cells, and wherein the CAR+CD20+ cells are susceptible to antibody-dependent cellular cytotoxicity mediated by NK effector cells expressing a chimeric receptor with an extracellular CD16 Fc binding domain, a transmembrane domain, and a cytoplasmic domain with a 4-1BB costimulatory domain and a CD3ζ primary signaling domain at an effector to target ratio of at least 1:1 during a 48 hour incubation in the presence of 1 μg/ml rituximab.

57. The cell population of claim 56, wherein at least 50% of the cells of the cell population are CAR+CD20+ cells, and wherein the CAR+CD20+ cells are susceptible to antibody-dependent cellular cytotoxicity mediated by NK effector cells expressing a chimeric receptor with an extracellular CD16 Fc binding domain, a transmembrane domain, and a cytoplasmic domain with a 4-1BB costimulatory domain and a CD3ζ primary signaling domain at an effector to target ratio of at least 1:1 during a 48 hour incubation in the presence of 1 μg/ml rituximab.

58. The cell population of any one of claims 46-55, wherein at least 20% of the cells of the cell population are CAR+CD20+ cells, and wherein the CAR+CD20+ cells are susceptible to complement-dependent cytotoxicity mediated by 25% (v/v) baby rabbit complement in the presence of at least 1 μg/ml rituximab.

59. The cell population of any one of claim 58, wherein at least 80% of the cells of the cell population are CAR+CD20+ cells, and wherein the CAR+CD20+ cells are susceptible to complement-dependent cytotoxicity mediated by 25% (v/v) baby rabbit complement in the presence of at least 1 μg/ml rituximab.

60. A method of manufacturing a cellular composition comprising:
(a) obtaining a cell population comprising immune cells,
(b) introducing a first polynucleotide into the immune cells, and
(c) introducing a second polynucleotide into the immune cells at least about two days after introducing the first polynucleotide into the immune cells,
wherein
the first polynucleotide encodes a protein expression blocker (PEBL), and
the second polynucleotide encodes a chimeric antigen receptor (CAR).

61. The method of claim 60, wherein the PEBL comprises a first CD3-binding domain coupled to an intracellular targeting signal.

62. The method of claim 61, wherein the intracellular targeting signal comprises an ER retention sequence, a Golgi retention sequence, or a proteosome localizing sequence.

63. The method of claim 62, wherein the PEBL further comprises a linker sequence between the first CD3-binding domain and the intracellular targeting signal.

64. The method of claim 63, wherein the linker sequence comprises a sequence of SEQ ID NO: 135.

65. The method of any one of claims 62-64, wherein the ER retention sequence comprises KDEL (SEQ ID NO: 137), KKXX or KXD/E, wherein X is any amino acid.

66. The method of any one of claims 62-65, wherein the ER retention sequence comprises SEQ ID NO: 142.

67. The method of any one of claims 62-65, wherein the ER retention sequence comprises SEQ ID NO: 143.

68. The method of any one of claims 62-65, wherein the Golgi retention sequence comprises YQRL (SEQ ID NO: 147).

69. The method of any one of claims 60-68, wherein the PEBL downregulates cell surface expression of endogenous CD3 in the immune cells.

70. The method of any one of claims 60-69, wherein the CAR comprises a second CD3-binding domain coupled to a transmembrane domain, a costimulatory domain from a costimulatory protein involved in immune cell costimulation, and a cytoplasmic signaling domain comprising an immunoreceptor tyrosine-based activation motif.

71. The method of claim 70, wherein the transmembrane domain comprises SEQ ID NO: 100.

72. The method of claim 70 or 71, wherein the costimulatory domain comprises SEQ ID NO: 102.

73. The method of any one of claims 70-72, wherein the cytoplasmic signaling domain comprises SEQ ID NO: 103.

74. The method of any one of claims 70-73, wherein the first CD3-binding domain or the second CD3-binding domain binds to CD3γ or CD3δ.

75. The method of any one of claims 70-73, wherein the first CD3-binding domain or the second CD3-binding domain binds to CD3ε.

76. The method of claim 75, wherein the first CD3-binding domain and the second CD3-binding domain each comprise six complementarity determining regions (CDRs) from heavy chain (HC) and light chain (LC) variable domains of a UCHT1 antibody, wherein:
HC CDR1 comprises SEQ ID NO: 13 or SEQ ID NO: 31,
HC CDR2 comprises SEQ ID NO: 14 or SEQ ID NO: 32,
HC CDR3 comprises SEQ ID NO: 15 or SEQ ID NO: 33,
LC CDR1 comprises SEQ ID NO: 22 or SEQ ID NO: 40,
LC CDR2 comprises SEQ ID NO: 23 or SEQ ID NO: 41, and
LC CDR3 comprises SEQ ID NO: 24 or SEQ ID NO: 42.

77. The method of claim 75 or 76, wherein the first CD3-binding domain and the second CD3-binding domain each comprise sequences having at least 90% identity to SEQ ID NO: 1 and SEQ ID NO: 2.

78. The method of claim 75 or 76, wherein the first CD3-binding domain and the second CD3-binding domain each comprise SEQ ID NO: 1 and SEQ ID NO: 2.

79. The method of claim 75, wherein the first CD3-binding domain comprises six complementarity determining regions (CDRs) from heavy chain (HC) and light chain (LC) variable domains of a UCHT1 antibody, wherein:
the first CD3-binding domain comprises six complementarity determining regions (CDRs) from heavy chain (HC) and light chain (LC) variable domains of a UCHT1 antibody, wherein:
HC CDR1 comprises SEQ ID NO: 13 or SEQ ID NO: 31,
HC CDR2 comprises SEQ ID NO: 14 or SEQ ID NO: 32,
HC CDR3 comprises SEQ ID NO: 15 or SEQ ID NO: 33,
LC CDR1 comprises SEQ ID NO: 22 or SEQ ID NO: 40,
LC CDR2 comprises SEQ ID NO: 23 or SEQ ID NO: 41, and
LC CDR3 comprises SEQ ID NO: 24 or SEQ ID NO: 42; and
the second CD3-binding domain comprises six complementarity determining regions (CDRs) from heavy chain (HC) and light chain (LC) variable domains of a 28F11 antibody, wherein:
HC CDR1 comprises SEQ ID NO: 19 or SEQ ID NO: 37,
HC CDR2 comprises SEQ ID NO: 20 or SEQ ID NO: 38,
HC CDR3 comprises SEQ ID NO: 21 or SEQ ID NO: 39,
LC CDR1 comprises SEQ ID NO: 28 or SEQ ID NO:46,
LC CDR2 comprises SEQ ID NO: 29 or SEQ ID NO: 47, and
LC CDR3 comprises SEQ ID NO: 30 or SEQ ID NO: 48.

80. The method of claim 75 or 79, wherein the first CD3-binding domain and the second CD3-binding domain each comprise sequences having at least 90% identity to SEQ ID NO: 5 and SEQ ID NO: 6.

81. The method of claim 75 or 79, wherein the first CD3-binding domain and the second CD3-binding domain each comprise SEQ ID NO: 5 and SEQ ID NO: 6.

82. The method of any one of claims 60-81, wherein the second polynucleotide further comprises a kill gene.

83. The method of claim 82, wherein the kill gene encodes CD20 or a truncated fragment thereof.

84. The method of claim 83, wherein the CD20 fragment comprises SEQ ID NO: 124.

85. The method of any one of claims 60-84, wherein a retroviral vector comprises the first polynucleotide.

86. The method of any one of claims 60-85, wherein a retroviral vector comprises the second polynucleotide.

87. The method of any one of claims 60-86, wherein a lentiviral vector comprises the first polynucleotide.

88. The method of any one of claims 60-87, wherein a lentiviral vector comprises the second polynucleotide.

89. The method of any one of claims 60-88, wherein the cell population comprises peripheral blood mononuclear cells.

90. The method of any one of claims 60-89, wherein at least 80% of the cells of the immune cell population are T cells.

91. The method of any one of claims 60-90, wherein at least 40% of the cells of the immune cell population are CD4-positive T cells.

92. The method of any one of claims 60-91, wherein at least 40% of the cells of the immune cell population are CD8-positive T cells.

93. The method of any one of claims 60-92, wherein the second polynucleotide is introduced about two days after introducing the first polypeptide into the immune cells.

94. The method of any one of claims 60-93, wherein the immune cells are activated before the introducing a first polynucleotide into the immune cells.

95. The method of any one of claims 60-94, wherein the activating comprises contacting the immune cells with an anti-CD3 antibody and an anti-CD28 antibody.

96. The method of any one of claims 60-95, wherein the activating comprises contacting the immune cells with a polymeric nanomatrix conjugated to anti-CD3 antibodies and anti-CD28 antibodies.

97. The method of any one of claims 60-96, further comprising culturing the cells in a growth media, wherein the cell population expands at least 10-fold after 10 or less days of growth.

98. The method of claim 97, wherein the culturing comprising contacting the cells to a gas permeable membrane.

99. The method of claim 97 or 98, further comprising harvesting the cell population and cryopreserving the cell population.

100. A cell population manufactured by the method of any one of claims 60-99.

101. A therapeutic composition comprising the cell population of any one of claims 46-58 or claim 100 and a pharmaceutically acceptable excipient.

102. A method of treating a T cell disease in a subject comprising administering the therapeutic composition of claim 101 to the subject.

103. The method of claim 102, wherein the T cell disease comprises a T cell lymphoma.

104. The method of claim 103, wherein the T cell lymphoma comprises peripheral T cell lymphoma.

105. The method of claim 102, wherein the T cell disease comprises a T cell leukemia.

106. The method of claim 102, wherein the T cell disease comprises an autoimmune disease.

107. The method of any one of claims 102-106, wherein graft versus host disease in the subject is reduced compared to an identical subject treated with a therapeutic composition comprising a cell population comprising immune cells expressing the CAR and not expressing the PEBL.

108. The method of any one of claims 102-107, further comprising administering a trigger that activates the kill gene.

109. The method of claim 108, wherein the trigger that activates the kill gene comprises an anti-CD20 antibody.

110. The method of claim 109, wherein the anti-CD20 antibody comprises rituximab.


SEQUENCE LISTING (161 sequences)

SEQ ID NO: 1 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 2 - protein, 107 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 3 - protein, 120 aa, synthetic construct
EVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSA

SEQ ID NO: 4 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 5 - protein, 118 aa, synthetic construct [sequence omitted]

SEQ ID NO: 6 - protein, 108 aa, synthetic construct [sequence omitted]

SEQ ID NO: 7 - protein, 120 aa, synthetic construct
EVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSA

SEQ ID NO: 8 - protein, 106 aa, synthetic construct
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELK

SEQ ID NO: 9 - protein, 120 aa, synthetic construct
QVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSS

SEQ ID NO: 10 - protein, 106 aa, synthetic construct
DIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIK

SEQ ID NO: 11 - protein, 119 aa, synthetic construct
QVQLQQSGAELARPGASVKMSCKASGYTFTSYTMHWVKQRPGQGLEWIGYINPSSGYTNYNQKFKDKATLTADKSSSTAYMQLSSLTSEDSAVYYCARWRDAYYAMDYWGQGTSVTVSS

SEQ ID NO: 12 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIK

SEQ ID NO: 13 - protein, 5 aa, synthetic construct
GYTMN

SEQ ID NO: 14 - protein, 17 aa, synthetic construct
LINPYKGVSTYNQKFKD

SEQ ID NO: 15 - protein, 13 aa, synthetic construct
SGYYGDSDWYFDV

SEQ ID NO: 16 - protein, 5 aa, synthetic construct
RYTMH

SEQ ID NO: 17 - protein, 17 aa, synthetic construct
YINPSRGYTNYNQKFKD

SEQ ID NO: 18 - protein, 10 aa, synthetic construct
YYDDHYCLDY

SEQ ID NO: 19 - protein, 5 aa, synthetic construct
GYGMH

SEQ ID NO: 20 - protein, 17 aa, synthetic construct
VIWYDGSKKYYVDSVKG

SEQ ID NO: 21 - protein, 9 aa, synthetic construct
QMGYWHFDL

SEQ ID NO: 22 - protein, 11 aa, synthetic construct
RASQDIRNYLN

SEQ ID NO: 23 - protein, 7 aa, synthetic construct
YTSRLHS

SEQ ID NO: 24 - protein, 9 aa, synthetic construct
QQGNTLPWT

SEQ ID NO: 25 - protein, 10 aa, synthetic construct
SASSSVSYMN

SEQ ID NO: 26 - protein, 7 aa, synthetic construct
DTSKLAS

SEQ ID NO: 27 - protein, 9 aa, synthetic construct
QQWSSNPFT

SEQ ID NO: 28 - protein, 11 aa, synthetic construct
RASQSVSSYLA

SEQ ID NO: 29 - protein, 7 aa, synthetic construct
DASNRAT

SEQ ID NO: 30 - protein, 10 aa, synthetic construct
QQRSNWPPLT

SEQ ID NO: 31 - protein, 7 aa, synthetic construct
GYSFTGY

SEQ ID NO: 32 - protein, 6 aa, synthetic construct
NPYKGV

SEQ ID NO: 33 - protein, 13 aa, synthetic construct
SGYYGDSDWYFDV

SEQ ID NO: 34 - protein, 7 aa, synthetic construct
GYTFTRY

SEQ ID NO: 35 - protein, 6 aa, synthetic construct
NPSRGY

SEQ ID NO: 36 - protein, 10 aa, synthetic construct
YYDDHYCLDY

SEQ ID NO: 37 - protein, 7 aa, synthetic construct
GFKFSGY

SEQ ID NO: 38 - protein, 6 aa, synthetic construct
WYDGSK

SEQ ID NO: 39 - protein, 9 aa, synthetic construct
QMGYWHFDL

SEQ ID NO: 40 - protein, 11 aa, synthetic construct
RASQDIRNYLN

SEQ ID NO: 41 - protein, 7 aa, synthetic construct
YTSRLHS

SEQ ID NO: 42 - protein, 9 aa, synthetic construct
QQGNTLPWT

SEQ ID NO: 43 - protein, 10 aa, synthetic construct
SASSSVSYMN

SEQ ID NO: 44 - protein, 7 aa, synthetic construct
DTSKLAS

SEQ ID NO: 45 - protein, 9 aa, synthetic construct
QQWSSNPFT

SEQ ID NO: 46 - protein, 11 aa, synthetic construct
RASQSVSSYLA

SEQ ID NO: 47 - protein, 7 aa, synthetic construct
DASNRAT

SEQ ID NO: 48 - protein, 10 aa, synthetic construct
QQRSNWPPLT

SEQ ID NO: 49 - protein, 5 aa, synthetic construct
SYVMH

SEQ ID NO: 50 - protein, 17 aa, synthetic construct
YINPYNDVTKYNEKFKG

SEQ ID NO: 51 - protein, 11 aa, synthetic construct
GSYYDYDGFVY

SEQ ID NO: 52 - protein, 5 aa, synthetic construct
SYVMH

SEQ ID NO: 53 - protein, 17 aa, synthetic construct
YINPYNDVTKYNEKFKG

SEQ ID NO: 54 - protein, 11 aa, synthetic construct
GSYYDYDGFVY

SEQ ID NO: 55 - protein, 4 aa, synthetic construct
SYTM

SEQ ID NO: 56 - protein, 17 aa, synthetic construct
YINPSSGYTNYNQKFKD

SEQ ID NO: 57 - protein, 10 aa, synthetic construct
WRDAYYAMDY

SEQ ID NO: 58 - protein, 10 aa, synthetic construct
SATSSVSYMH

SEQ ID NO: 59 - protein, 7 aa, synthetic construct
DTSKLAS

SEQ ID NO: 60 - protein, 9 aa, synthetic construct
QQWSSNPLT

SEQ ID NO: 61 - protein, 10 aa, synthetic construct
SATSSVSYMH

SEQ ID NO: 62 - protein, 7 aa, synthetic construct
DTSKLAS

SEQ ID NO: 63 - protein, 9 aa, synthetic construct
QQWSSNPLT

SEQ ID NO: 64 - protein, 10 aa, synthetic construct
SASSSVSYMH

SEQ ID NO: 65 - protein, 7 aa, synthetic construct
DTSKLAS

SEQ ID NO: 66 - protein, 9 aa, synthetic construct
QQWSSNPFT

SEQ ID NO: 67 - protein, 7 aa, synthetic construct
GYKFTSY

SEQ ID NO: 68 - protein, 6 aa, synthetic construct
NPYNDV

SEQ ID NO: 69 - protein, 11 aa, synthetic construct
GSYYDYDGFVY

SEQ ID NO: 70 - protein, 7 aa, synthetic construct
GYKFTSY

SEQ ID NO: 71 - protein, 6 aa, synthetic construct
NPYNDV

SEQ ID NO: 72 - protein, 11 aa, synthetic construct
GSYYDYDGFVY

SEQ ID NO: 73 - protein, 7 aa, synthetic construct
GYTFTSY

SEQ ID NO: 74 - protein, 6 aa, synthetic construct
NPSSGY

SEQ ID NO: 75 - protein, 10 aa, synthetic construct
WRDAYYAMDY

SEQ ID NO: 76 - protein, 10 aa, synthetic construct
SATSSVSYMH

SEQ ID NO: 77 - protein, 7 aa, synthetic construct
DTSKLAS

SEQ ID NO: 78 - protein, 9 aa, synthetic construct
QQWSSNPLT

SEQ ID NO: 79 - protein, 10 aa, synthetic construct
SATSSVSYMH

SEQ ID NO: 80 - protein, 7 aa, synthetic construct
DTSKLAS

SEQ ID NO: 81 - protein, 9 aa, synthetic construct
QQWSSNPLT

SEQ ID NO: 82 - protein, 10 aa, synthetic construct
SASSSVSYMH

SEQ ID NO: 83 - protein, 7 aa, synthetic construct
DTSKLAS

SEQ ID NO: 84 - protein, 9 aa, synthetic construct
QQWSSNPFT

SEQ ID NO: 85 - protein, 20 aa, synthetic construct
GGGGSGGGGSGGGGSGGGGS

SEQ ID NO: 86 - protein, 30 aa, synthetic construct
  VARIANT 1..30: note = This sequence may encompass 1-6 GGGGS repeating units
GGGGSGGGGSGGGGSGGGGSGGGGSGGGGS

SEQ ID NO: 87 - protein, 20 aa, synthetic construct
  VARIANT 1..20: note = This sequence may encompass 1-10 GS repeating units
GSGSGSGSGSGSGSGSGSGS

SEQ ID NO: 88 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 89 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 90 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 91 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 92 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 93 - protein, 15 aa, synthetic construct
GGGGSGGGGSGGGGS

SEQ ID NO: 94 - protein, 10 aa, synthetic construct
GGGGSGGGGS

SEQ ID NO: 95 - protein, 495 aa, synthetic construct
MALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 474 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 21 aa, synthetic construct
MALPVTALLLPLALLLHAARP

SEQ ID NO: 98 - protein, 249 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 99 - protein, 47 aa, synthetic construct
KPTTTPAPRPPTPAPTIASQPLSLRPEACRPAAGGAVHTRGLDFACD

SEQ ID NO: 100 - protein, 24 aa, synthetic construct
IYIWAPLAGTCGVLLLSLVITLYC

SEQ ID NO: 101 - protein, 71 aa, synthetic construct
KPTTTPAPRPPTPAPTIASQPLSLRPEACRPAAGGAVHTRGLDFACDIYIWAPLAGTCGVLLLSLVITLYC

SEQ ID NO: 102 - protein, 42 aa, synthetic construct
KRGRKKLLYIFKQPFMRPVQTTQEEDGCSCRFPEEEEGGCEL

SEQ ID NO: 103 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 493 aa, synthetic construct
MALPVTALLLPLALLLHAARPQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSEVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 472 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSEVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 247 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSEVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSA

SEQ ID NO: 107 - protein, 492 aa, synthetic construct
MALPVTALLLPLALLLHAARPEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 471 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 246 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSS

SEQ ID NO: 110 - protein, 492 aa, synthetic construct
MALPVTALLLPLALLLHAARPQIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELKGGGGSGGGGSGGGGSGGGGSEVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 471 aa, synthetic construct
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELKGGGGSGGGGSGGGGSGGGGSEVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 246 aa, synthetic construct
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELKGGGGSGGGGSGGGGSGGGGSEVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSA

SEQ ID NO: 113 - protein, 492 aa, synthetic construct
MALPVTALLLPLALLLHAARPDIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIKGGGGSGGGGSGGGGSGGGGSQVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 471 aa, synthetic construct
DIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIKGGGGSGGGGSGGGGSGGGGSQVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 246 aa, synthetic construct
DIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIKGGGGSGGGGSGGGGSGGGGSQVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSS

SEQ ID NO: 116 - protein, 493 aa, synthetic construct
MALPVTALLLPLALLLHAARPQIVLTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIKRAGGGGSGGGGSGGGGSGGGGSQVQLQQSGAELARPGASVKMSCKASGYTFTSYTMHWVKQRPGQGLEWIGYINPSSGYTNYNQKFKDKATLTADKSSSTAYMQLSSLTSEDSAVYYCARWRDAYYAMDYWGQGTSVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 472 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIKRAGGGGSGGGGSGGGGSGGGGSQVQLQQSGAELARPGASVKMSCKASGYTFTSYTMHWVKQRPGQGLEWIGYINPSSGYTNYNQKFKDKATLTADKSSSTAYMQLSSLTSEDSAVYYCARWRDAYYAMDYWGQGTSVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 247 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIKRAGGGGSGGGGSGGGGSGGGGSQVQLQQSGAELARPGASVKMSCKASGYTFTSYTMHWVKQRPGQGLEWIGYINPSSGYTNYNQKFKDKATLTADKSSSTAYMQLSSLTSEDSAVYYCARWRDAYYAMDYWGQGTSVTVSS

SEQ ID NO: 119 - protein, 22 aa, synthetic construct
RAGGGGSGGGGSGGGGSGGGGS

SEQ ID NO: 120 - protein, 780 aa, synthetic construct
MALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIE

SEQ ID NO: 121 - protein, 759 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIE

SEQ ID NO: 122 - protein, 22 aa, synthetic construct
GSGATNFSLLKQAGDVEENPGP

SEQ ID NO: 123 - protein, 297 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 124 - protein, 263 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIE

SEQ ID NO: 125 - protein, 495 aa, synthetic construct; identical to SEQ ID NO: 95

SEQ ID NO: 126 - protein, 474 aa, synthetic construct; identical to SEQ ID NO: 96

SEQ ID NO: 127 - DNA, 577 bp, synthetic construct
  source 1..577: mol_type = other DNA
cgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatacc

SEQ ID NO: 128 - protein, 801 aa, synthetic construct
MALPVTALLLPLALLLHAARPMTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEGSGATNFSLLKQAGDVEENPGPMALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 780 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEGSGATNFSLLKQAGDVEENPGPMALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 549 aa, synthetic construct
MALPVTALLLPLALLLHAARPGGGGSCPYSNPSLCSGGGGSCPYSNPSLCSGGGGSCPYSNPSLCGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 528 aa, synthetic construct
GGGGSCPYSNPSLCSGGGGSCPYSNPSLCSGGGGSCPYSNPSLCGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 54 aa, synthetic construct
GGGGSCPYSNPSLCSGGGGSCPYSNPSLCSGGGGSCPYSNPSLCGGGGSGGGGS

SEQ ID NO: 133 - DNA, 1184 bp, synthetic construct
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 134 - DNA, 384 bp, synthetic construct
  source 1..384: mol_type = other DNA
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcc

SEQ ID NO: 135 - protein, 22 aa, synthetic construct
GGGGSGGGGSGGGGSGGGGSAE

SEQ ID NO: 136 - protein, 60 aa, synthetic construct
  VARIANT 1..60: note = This sequence may encompass 2-12 GGGGS repeating units
GGGGSGGGGSGGGGSGGGGSGGGGSGGGGSGGGGSGGGGSGGGGSGGGGSGGGGSGGGGS

SEQ ID NO: 137 - protein, 4 aa, synthetic construct
KDEL

SEQ ID NO: 138 - protein, 5 aa, synthetic construct
GGGGS

SEQ ID NO: 139 - protein, 10 aa, synthetic construct
EQKLISEEDL

SEQ ID NO: 140 - protein, 6 aa, synthetic construct
AEKDEL

SEQ ID NO: 141 - protein, 14 aa, synthetic construct
EQKLISEEDLKDEL

SEQ ID NO: 142 - protein, 26 aa, synthetic construct
GGGGSGGGGSGGGGSGGGGSAEKDEL

SEQ ID NO: 143 - protein, 36 aa, synthetic construct
EQKLISEEDLGGGGSGGGGSGGGGSGGGGSAEKDEL

SEQ ID NO: 144 - protein, 26 aa, synthetic construct
DDHYCLDYWGQGTTLTVSSAAEKDEL

SEQ ID NO: 145 - protein, 15 aa, synthetic construct
KYKSRRSFIEEKKMP

SEQ ID NO: 146 - protein, 83 aa, synthetic construct
TTTPAPRPPTPAPTIASQPLSLRPEACRPAAGGAVHTRGLDFACDIYIWAPLAGTCGVLLLSLVITLYKYKSRRSFIDEKKMP

SEQ ID NO: 147 - protein, 4 aa, synthetic construct
YQRL

SEQ ID NO: 148 - protein, 4 aa, synthetic construct
YGRL

SEQ ID NO: 149 - protein, 4 aa, synthetic construct
YKGL

SEQ ID NO: 150 - protein, 4 aa, synthetic construct
PEST

SEQ ID NO: 151 - protein, 40 aa, synthetic construct
SHGFPPEVEEQDDGTLPMSCAQESGMDRHPAACASARINV

SEQ ID NO: 152 - protein, 306 aa, synthetic construct
MALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSEQKLISEEDLGGGGSGGGGSGGGGSGGGGSAEKDEL

SEQ ID NO: 153 - protein, 285 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSEQKLISEEDLGGGGSGGGGSGGGGSGGGGSAEKDEL

SEQ ID NO: 154 - protein, 304 aa, synthetic construct
MALPVTALLLPLALLLHAARPQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSEVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAEQKLISEEDLGGGGSGGGGSGGGGSGGGGSAEKDEL

SEQ ID NO: 155 - protein, 283 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSEVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAEQKLISEEDLGGGGSGGGGSGGGGSGGGGSAEKDEL

SEQ ID NO: 156 - protein, 303 aa, synthetic construct
MALPVTALLLPLALLLHAARPEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSEQKLISEEDLGGGGSGGGGSGGGGSGGGGSAEKDEL

SEQ ID NO: 157 - protein, 282 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSEQKLISEEDLGGGGSGGGGSGGGGSGGGGSAEKDEL

SEQ ID NO: 158 - protein, 303 aa, synthetic construct
MALPVTALLLPLALLLHAARPQIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELKGGGGSGGGGSGGGGSGGGGSEVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSAEQKLISEEDLGGGGSGGGGSGGGGSGGGGSAEKDEL

SEQ ID NO: 159 - protein, 282 aa, synthetic construct
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELKGGGGSGGGGSGGGGSGGGGSEVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSAEQKLISEEDLGGGGSGGGGSGGGGSGGGGSAEKDEL

SEQ ID NO: 160 - protein, 303 aa, synthetic construct
MALPVTALLLPLALLLHAARPDIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIKGGGGSGGGGSGGGGSGGGGSQVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSSEQKLISEEDLGGGGSGGGGSGGGGSGGGGSAEKDEL

SEQ ID NO: 161 - protein, 282 aa, synthetic construct
DIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIKGGGGSGGGGSGGGGSGGGGSQVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSSEQKLISEEDLGGGGSGGGGSGGGGSGGGGSAEKDEL